SEQUENCE LISTING (40 sequences)

SEQ ID NO: 1 - DNA, 3376 bp, Lymphocytic choriomeningitis virus
  misc_feature 1..3376: note = LCMV strain WE
  source 1..3376: mol_type = genomic DNA
gcgcaccggggatcctaggctttttggattgcgctttcctttaggacaattgggtgctggattctatccaataaaaggatgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctgatcagcagcgcctccctgactctccacctcgaaagaggtggagagtcagggaggcccagtgggtcttagagtgtcacaacattgggtcctctgaagattaaatcatgtggcaggatgttgtgaacggtttttagatcagggagtcttgccttggaagcactctcaaaaatgatgcagtccatgagtgcacagtgtggggtgatctctttcttctttttgtctctcactaccccagtgtgcattttgcatagccagccatatttgtcccacactttatcttcatattctcttgaggcctccttggtcatctcaacatcaatgagttttatgtcccttctattttgtgagtccagaagctttctgatgtcatcagaaccttgacagctcaaaaccatcccttgtgggagagcacctataactgatgaggtcagcccaggctgtgcattgaagagatcagcaagatccatgccgtgtgaatactttgagtcctgcttgaattgcttctggtccgtaggttccctgtaaaaatgtatgaattgcccattttgtggttggaatattgctatctccactggatcattgaacctgccctcaatgtcaatccatgtgggagcattgggatcaatccctcccatcaagtctttcaacagcattgtctgactgtaactcaagcccacctgaggtgggcctgctgctccaggcactggcctagatgagttggcaacaagtttttcatttgtgagatcaattgttgtgttctcccatgctctccccacaactgacgttctacaggctatgtatggccatccttcacctgaaagacagactttataaaggatgttttcatagggatttctatctccaacttgatctgagacaaacatgttgagtttcttcttggccccgaggactgcttttaggagatcctcactgttgctcggtttgatcaagatagattccagcatgttccctccatctagcagagctgcccccgctttcacagctgcaccaagactgaaattataaccagagatattgatactagattgctgttcagtaatgacccccagaactgggtgtttatcctttagcctttcaagatcactgagattcgggtatttgactgtgtaaagtaagccaaggtctgtgagtgcctgcacaacatcattgagtggggtctgtgactgttttgccatgcaagccattgtcaggcttggcattgtgccgaactgattgttcagaagtgatgagtccttcacatcccaaacccttactacaccacttgcaccctgctgaggtcttctcatcccaaccatttgcagtatttgggatctttgatcaagttgttgtgctgtcaaatttcccatgtagactccagaagcttgaggcctctcagttctcataattttggccttcagcttctcaagatcagctgcaagggtcatcaattcctctgcactaagtcttcccactttcagaacatttttctttgatgtagacttcagatcaacaagagaatgcacagtctggttaagactcctgagtctctgcaagtctttatcatccctcctttcctttctcatgatcctctgaacgttgctgacttcagaaaagtccaacccatttagaagactggttgcgtccttgatgacggcagcctttacatctgatgtaaaactctgcaactccctcctcaacgcctgtgtccactgaaagcttttgacttctttggacaaagacattttgtcacacaatgaatttccaaataaaagcgcaattaaatgcctaggatccactgtgcg

SEQ ID NO: 2 - DNA, 7236 bp, Lymphocytic choriomeningitis virus
  misc_feature 1..7236: note = LCMV strain WE
  source 1..7236: mol_type = genomic DNA
gcgcaccggggatcctaggcgtttagttgcgctgctttattgcacagcttcactctgctaaaccatcaggaactgaccgatcatcagtcatgggccaaggcaagtccaaagaagaaagggacaccagcaatacaggcagagcagagcttttgccagacaccacctatcttggtcctctaaattgtaaatcatgttggcagaaatttgacagcttggttagatgccatgaccactatctttgcagacactgtctgaatctcctgctgtcagtttccgacagatgtcctctctgtaagtatccactgccaaccaaactgaaggtgtcaacagtcccaagctccccacctccctatgaggagtgacgccccagacctcgacacaacaggccacccaacacagaaaaaccacacacaaaccggcacacacagacacagcaaccagcacaaagcgcacgaaaacacacacacacacacacacacaccctcacccacgcgcccccccgcaccgccgggggggcgcccccccggggggcagccccccgggggcccgggcagggccccgcggagatgccggctagtcgatctcctcggccacccgctccgccagccaatcgtcacaggatttccccttgagtctgaacctgcccccagttgtctcatacattacagtgttctttgacctgctgaagcagagcctacaatctctgaaggtgaaccaatctggcacaaaagataatggtgtcagcatgacggatctgtccatgcagagctcctgaagaactgtacttatttctgactccatcaagtgctcaccagttctgaatcggtgcaggaggaggctgccgaggacatcaccaatttttccacagccctcgagctctgccagaaagaccctcatgtcctttgtctccaatttcacagagatattctgaacaaagttcctcccctcaaagagggcacccttccttatggtcagaggcacaggttcccactcaggccctatcctctcaaagtcagtagatttgatctcattcagaatccttctagaacctattagttcaagttctagtgaatcaccaagtgtcaaagggtcttccatgtaatcttcaaactcttcagacctaatgtcaaagactccatcattcgctttaaagatggggtctgtcctcaacagattgagacactcatctaataatctcctgtcaacctcacctttgaagaaatgagagcacgataagtaatcagctacacctggactctgtaattggcatttaaccacaaagatcaatgaaaatttcctcaagcagtcagtgttgttctggttgtgtgtgaaatccactgtgattgagaactccaatatcccctctgtgctgctgagcatgtaatcccacaaatctcttaaagatttaaatgcttttggatctgtcaaaccctgcttaattaacatagcggcattacacacaacatcccccacacgataagagaaccaaccaaaaccaaactgcaagtcattcctaaacatgggcctctctattttcttgttcaccactttcaaaataaatgattggaagggtcccagtgcttcagcgccatcttcagatggcatcatgtccttatgggggaaccatgaaaagttacctaaggtcctagctgttgcaacaaactctcgtacaaatgattcgaaatacacttgcttcaaaaagttcttgcagacatcccttgtgctgacaacaaattcatcaactaaacttgagtctgaccgttggtgagagttgacgagatcagaaaacagcacagtgtaatgttcgtccctcttccatttgacaacatgagaaatgagtgacaaagactctgagttgatgtcaatcagcacacaaaggtcaaggaatttaatcctgggactccatctcatattttttgagctcacatcagacatgaatgggagcaactgatcctcaaagattttggggtacagtcgcctcacagattgaattacatgattcaaactcactttatcctctagtagtctcgaactctcaggttttcttgctacataatcacatggatttaagtgcctaacagccgaattactctcgttctttcctttcacaggttctgctaaaacccaaacacccagctcaaaagagttgctcaatgagatgcaaatgtagtcccagagaaggggcctcaaaaggttgatgtggtcacggtgagcctctgggtgtctctgcttgtcgcaaatgtacaacatcacaccatcttgtttgtgaacccttgtgacatgttcatccatggtcagatcttcaagcacctttctgatatacatattttccctgtttttatttctcacacacctacttcctaaagtcttacagagacctataaagccggatgagacacaactctgaaaagctgatttgttgattgcctctgacaacagcttctgtgcacctcttgtgaacttactacaaagcttattttggagtgttttaatcaatgaggggatcttttcctcttgaaaggtcattactgatgggtaaaccactttttgtctcagaaccatcctcaatgggaacatttcattcagattcaaccaattgatgcttgccaactcattaagatcatcctcaagaccaagaatctctcccaattgaaggatggcctctttcttttccctgttgaagaggtctagaaaaaattcctcgttacattcaccgttcttgagtttgtgatgtagtcttcttacaagttttctcatgatctttgtttcactgggacacaattcttcaatgagtctttggattctataacctctagaaccatctaaccaatcctttacatcagtgtgagtgtttaacaagaatggatctaaagggaaattagcatatttcaagagatccagtgtcctcctctggatacagtttactaaaaaaactggaactccatttgctatagcttgatcagcaatcgtatctattgtctcacaaagttgatgcggttctttacacttaacattgtgtagtgccgcagacacaaatttcgtgaggagagggacctcctccccccacacataaaatctggatttgaattccgctgcaaaccgcccaaccacactttttggactgatgaacttgttcaacaaaccacttaagtgagagtggaattccagcaagacaaggacttcttctgggtcactatcaactaagtcactcaatctcttgtcaaataaagtgatctgatcatcacttgatgtataggcttctggcctctctccaaaaatgacaccaatacaataattgataaacctttcactaattaaaccgtaaaaatcagaagcattgtgtaggatcccctgtcccatgtcaatgagactagatatgtgagatggcaccacccccatttcaaaatactctctaaagattctctccgtaacagtcgttcctgaccctttgagaagcttgagttttgatttaacatatgatttcatcattgcattcacaacagggaaagggacttcaacaagtttatgcatatgccaagtcaacaaggtgctaacatgatcttttccagaacgcacgtactgatcatcacccagtttgagattttggagaagcattaagaataggaatgggcacatcattggtccccatttgctgtgatccatgctgtagtttaacaacccttctctcacattgatagtcattgacaagattgcattttcaaactctttgtcattgtttaaacaagatcctgaaaagaaactagaaaaggattcaaaataatcttctaccaatcttgtgaacatttttgtcctcaaatccccaatgtaaagctctctatttcccccaacctgctctttgtatgacaatgcaaattttagtctcccagagtcagggccaactgaagtgtatgatgttggtgattcttctgagtaaaaacacagattctttagagcagcactcatgcattttgtcaatgaaagggccttacttagagattcagaattgctttccctttcactaattcttacatcttcttccaatttgtcccagtcaaatttaaaatttaagctctgcctctgcatgtgtctgtacttccctgaatacgcatttgcattcatctgtaacaagatcatcttcatacatgagaaccaatcaccctctgagaagaactttctacagaggtttcttgccatctcatccagaccacattgttctttgacagctgatgtaaaatacaatggtgacagttctgttgaaccttcaacagcctcacaaataaacctcatgtcgtcattggtgagacaggatggatcaaagtcctctactagatgaaatgaaatttctgataaaatgactttcctcagataagctcttttacctgacaaaataatctgaaggtggtgtagtccttttgtgttcttgaattctccctccccttgcccttcattaagcctagtgcctctattataccgtgttattgtggagctgaccttatcttctaaactcctgaaaaaacttgtctcctcctccccctcgaaacacacatctaccggatcatcttccaatttgtctacttctgttttcctggaaccgatgactaatctagagacaagctttgagaccttatattcgtaatctgagtgtctcagcttatacttttgtttcctcatgaagccctctgtaatttggctcacagcactaacaagcaatttgttaaaatcatactccaaaagccgttctccattcagatgtttattgacaaccacacttttgttgcttgcgagatctaatgctgtcgcacatccagagttggtcatggggtccagattgttgagccttttctccccttttagaattaaggtgccattgttgaatgaagataccatcatgctaaagatctctaaattaacaccaggggttgaatgctgacaatcaatatctttaccggtgaacttcttcatttgttcataaaaaacacattcctcttcgggtgtgattgtttccttagggttgacaaagaaaccaaactcactcttgggctcaaagaacttttcaaaacattttatctgatctgtcaatctatcaggggtctcctttgtgatcaaatgacacaggtatgacacattcaacataaacttgaattttgcactcaataacaccttttcaccagtaccaaaaattgtcctaatcaaaaaccgaagcagcctgtacaccactttctcggcaggtgtgattagatcctccttcaacttatccattaatgaggtcgatgaaaagtctgagacgatggccatcactaaatacctaatattttgaacctgcttttgatttctctttgttgggttggtgagcatcagtaggatcaatgtccttagtgcagtctcaatgtcaataagagaatcttttaagtcagggcatgacctaatccatgaaatcattgtgtctatcatgttgtgcaacacctcatctgaaaaaattggtaaaaagaacctttttggatctgcgtaaaaggaaatcaagtgaccatccggaccttgtatggaataacacctcgatgattctccagttttctgatatagtagatgatactcttcggaatccagttttattatctggcaaaacacctctttgcactccaccacttgatatctcacagaccccagttggttctgtctcaaccttgcaactgagcttgttttcatgctgtttgttagagccaaacaaacagatgacagttttctcaaactctgtaaacccctcaattgctctatgttaggctggaaagtgtaatcttcaaattttgtgtaatgcattacaggatgagtcccggctctcataaagttctcaaattcagcaaatggtatatggcattcttgcacgaggcattctgatagtccgtaatgctcaaaactaagtcccactgttgataggcatttctggatttttgctatgaactcatttatggataccctaaaaagctcatcaagagatgcctttttgtgcactcttgattttcttctgttcttcaaaagtctcatgaattcttctttggtactttgaaggctcacaagcctatcattcacactactgtagcaacaaccaacccaatgtttatcattttccaaccctttggaaattgactgcttaataagtgaagaaagacataagacatctaagttcagcagctgtctccttctagtgtttaataattttaaacttttcaccttgttcaacatagagagtagcctttcatactcagtgttagcatcgcttcctctctcatgcccatgggtctctgctgtaatgaacctcattaaggggcaggattcaactgcctctttgctcaacattaatatgtcatcattatcagtaagggtttcataaagttcagagaattccttgattaagcccccagggctgaccttttggaaattccttttgagtttcccatttaacaggtccctcctaaacctgccaaaaaaggaatttatgccaaagaccacatcatcgcaactcatgttggggttgacaccatcgtggcacattttcataatctcatcgttatgaaatgatcttgcgtctttcaaaattttcattgagtccatgccggaacgtttgtcaacagtggtcttaagagactcacagagcctgaagtattcagactcctcaaaaactttctcatcttgactagaatactccaggagtttaaataggaggtctctgaacttaaaattcacccattctggcatgaaactgttatcataatcacaacgaccatccactattggaaccagtgtgatgcctgcaacagcaagatcttctttgatgcatgctagtttgttggtgtcctcaatgaacttcttttcaaaactagctggtgtgcttctaacaaaacattcaagaagaataagggaattgtcaatcaacttataaccatcagggatgatgagtggcagtcctggacacacaatcccagagtctattaggattgtttcaacagatttgtcatcgtggttgtgtatgcaaccacttttgtctgcactgtctatctctatacagcgtgataacaatttgagtccctcaattagcaccattctgggttctctttgtcccaggaagttgagtttttgccttgacagcctctcgtcctgttctatgtaatttagacacaactctctcaaatctgcaatagtttcatccattgcgcatcaaaaagcctaggatcctcggtgcgc

SEQ ID NO: 3 - DNA, 1497 bp, Lymphocytic choriomeningitis virus
  misc_feature 1..1497: note = LCMV strain WE
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 4 - protein, 498 aa, Lymphocytic choriomeningitis virus
  REGION 1..498: note = misc_feature - LCMV strain WE
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 5 - DNA, 1677 bp, Lymphocytic choriomeningitis virus
  misc_feature 1..1677: note = LCMV strain WE
  source 1..1677: mol_type = genomic DNA
atgtctttgtccaaagaagtcaaaagctttcagtggacacaggcgttgaggagggagttgcagagttttacatcagatgtaaaggctgccgtcatcaaggacgcaaccagtcttctaaatgggttggacttttctgaagtcagcaacgttcagaggatcatgagaaaggaaaggagggatgataaagacttgcagagactcaggagtcttaaccagactgtgcattctcttgttgatctgaagtctacatcaaagaaaaatgttctgaaagtgggaagacttagtgcagaggaattgatgacccttgcagctgatcttgagaagctgaaggccaaaattatgagaactgagaggcctcaagcttctggagtctacatgggaaatttgacagcacaacaacttgatcaaagatcccaaatactgcaaatggttgggatgagaagacctcagcagggtgcaagtggtgtagtaagggtttgggatgtgaaggactcatcacttctgaacaatcagttcggcacaatgccaagcctgacaatggcttgcatggcaaaacagtcacagaccccactcaatgatgttgtgcaggcactcacagaccttggcttactttacacagtcaaatacccgaatctcagtgatcttgaaaggctaaaggataaacacccagttctgggggtcattactgaacagcaatctagtatcaatatctctggttataatttcagtcttggtgcagctgtgaaagcgggggcagctctgctagatggagggaacatgctggaatctatcttgatcaaaccgagcaacagtgaggatctcctaaaagcagtcctcggggccaagaagaaactcaacatgtttgtctcagatcaagttggagatagaaatccctatgaaaacatcctttataaagtctgtctttcaggtgaaggatggccatacatagcctgtagaacgtcagttgtggggagagcatgggagaacacaacaattgatctcacaaatgaaaaacttgttgccaactcatctaggccagtgcctggagcagcaggcccacctcaggtgggcttgagttacagtcagacaatgctgttgaaagacttgatgggagggattgatcccaatgctcccacatggattgacattgagggcaggttcaatgatccagtggagatagcaatattccaaccacaaaatgggcaattcatacatttttacagggaacctacggaccagaagcaattcaagcaggactcaaagtattcacacggcatggatcttgctgatctcttcaatgcacagcctgggctgacctcatcagttataggtgctctcccacaagggatggttttgagctgtcaaggttctgatgacatcagaaagcttctggactcacaaaatagaagggacataaaactcattgatgttgagatgaccaaggaggcctcaagagaatatgaagataaagtgtgggacaaatatggctggctatgcaaaatgcacactggggtagtgagagacaaaaagaagaaagagatcaccccacactgtgcactcatggactgcatcatttttgagagtgcttccaaggcaagactccctgatctaaaaaccgttcacaacatcctgccacatgatttaatcttcagaggacccaatgttgtgacactctaa

SEQ ID NO: 6 - protein, 558 aa, Lymphocytic choriomeningitis virus
  REGION 1..558: note = misc_feature - LCMV strain WE
MSLSKEVKSFQWTQALRRELQSFTSDVKAAVIKDATSLLNGLDFSEVSNVQRIMRKERRDDKDLQRLRSLNQTVHSLVDLKSTSKKNVLKVGRLSAEELMTLAADLEKLKAKIMRTERPQASGVYMGNLTAQQLDQRSQILQMVGMRRPQQGASGVVRVWDVKDSSLLNNQFGTMPSLTMACMAKQSQTPLNDVVQALTDLGLLYTVKYPNLSDLERLKDKHPVLGVITEQQSSINISGYNFSLGAAVKAGAALLDGGNMLESILIKPSNSEDLLKAVLGAKKKLNMFVSDQVGDRNPYENILYKVCLSGEGWPYIACRTSVVGRAWENTTIDLTNEKLVANSSRPVPGAAGPPQVGLSYSQTMLLKDLMGGIDPNAPTWIDIEGRFNDPVEIAIFQPQNGQFIHFYREPTDQKQFKQDSKYSHGMDLADLFNAQPGLTSSVIGALPQGMVLSCQGSDDIRKLLDSQNRRDIKLIDVEMTKEASREYEDKVWDKYGWLCKMHTGVVRDKKKKEITPHCALMDCIIFESASKARLPDLKTVHNILPHDLIFRGPNVVTL

SEQ ID NO: 7 - DNA, 273 bp, Lymphocytic choriomeningitis virus
  misc_feature 1..273: note = LCMV strain WE
  source 1..273: mol_type = genomic DNA
atgggccaaggcaagtccaaagaagaaagggacaccagcaatacaggcagagcagagcttttgccagacaccacctatcttggtcctctaaattgtaaatcatgttggcagaaatttgacagcttggttagatgccatgaccactatctttgcagacactgtctgaatctcctgctgtcagtttccgacagatgtcctctctgtaagtatccactgccaaccaaactgaaggtgtcaacagtcccaagctccccacctccctatgaggagtga

SEQ ID NO: 8 - protein, 90 aa, Lymphocytic choriomeningitis virus
  REGION 1..90: note = misc_feature - LCMV strain WE
MGQGKSKEERDTSNTGRAELLPDTTYLGPLNCKSCWQKFDSLVRCHDHYLCRHCLNLLLSVSDRCPLCKYPLPTKLKVSTVPSSPPPYEE

SEQ ID NO: 9 - DNA, 6630 bp, Lymphocytic choriomeningitis virus
  misc_feature 1..6630: note = LCMV strain WE
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaagaaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcatttcttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggaaaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcaggtcaaagaacactgtaatgtatgagacaactgggggcaggttcagactcaaggggaaatcctgtgacgattggctggcggagcgggtggccgaggagatcgactag

SEQ ID NO: 10 - protein, 2209 aa, Lymphocytic choriomeningitis virus
  REGION 1..2209: note = misc_feature - LCMV strain WE
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHFFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGKIGDVLGSLLLHRFRTGEHLMESEISTVLQELCMDRSVMLTPLSFVPDWFTFRDCRLCFSRSKNTVMYETTGGRFRLKGKSCDDWLAERVAEEID

SEQ ID NO: 11 - DNA, 3376 bp, Lymphocytic choriomeningitis virus
  misc_feature 1..3376: note = LCMV strain P52-WE
  source 1..3376: mol_type = genomic DNA
gcgcaccggggatcctaggctttttggattgcgctttcctttaggacaattgggtgctggattctatccaataaaaggatgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctatgagccagtgttggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctgatcagcagcgcctccctgactctccacctcgaaagaggtggagagtcagggaggcccagtgggtcttagagtgtcacaacattgggtcctctgaagattaaatcatgtggcaggatgttgtgaacggtttttagatcagggagtcttgccttggaagcactctcaaaaatgatgcagtccatgagtgcacagtgtggggtgatctctttcttctttttgtctctcactaccccagtgtgcattttgcatagccagccatatttgtcccacactttatcttcatattctcttgaggcctccttggtcatctcaacatcaatgagttttatgtcccttctattttgtgagtccagaagctttctgatgtcatcagaaccttgacagctcaaaaccatcccttgtgggagagcacctataactgatgaggtcagcccaggctgtgcattgaagagatcagcaagatccatgccgtgtgaatactttgagtcctgcttgaattgcttctggtccgtaggttccctgtaaaaatgtatgaattgcccattttgtggttggaatattgctatctccactggatcattgaacctgccctcaatgtcaatccatgtgggagcattgggatcaatccctcccatcaagtctttcaacagcattgtctgactgtaactcaagcccacctgaggtgggcctgctgctccaggcactggcctagatgagttggcaacaagtttttcatttgtgagatcaattgttgtgttctcccatgctctccccacaactgacgttctacaggctatgtatggccatccttcacctgaaagacagactttataaaggatgttttcatagggatttctatctccaacttgatctgagacaaacatgttgagtttcttcttggccccgaggactgcttttaggagatcctcactgttgctcggtttgatcaagatagattccagcatgttccctccatctagcagagctgcccccgctttcacagctgcaccaagactgaaattataaccagagatattgatactagattgctgttcagtaatgacccccagaactgggtgtttatcctttagcctttcaagatcactgagattcgggtatttgactgtgtaaagtaagccaaggtctgtgagtgcctgcacaacatcattgagtggggtctgtgactgttttgccatgcaagccattgtcaggcttggcattgtgccgaactgattgttcagaagtgatgagtccttcacatcccaaacccttactacaccacttgcaccctgctgaggtcttctcatcccaaccatttgcagtatttgggatctttgatcaagttgttgtgctgtcaaatttcccatgtagactccagaagcttgaggcctctcagttctcataattttggccttcagcttctcaagatcagctgcaagggtcatcaattcctctgcactaagtcttcccactttcagaacatttttctttgatgtagacttcagatcaacaagagaatgcacagtctggttaagactcctgagtctctgcaagtctttatcatccctcctttcctttctcatgatcctctgaacgttgctgacttcagaaaagtccaacccatttagaagactggttgcgtccttgatgacggcagcctttacatctgatgtaaaactctgcaactccctcctcaacgcctgtgtccactgaaagcttttgacttctttggacaaagacattttgtcacacaatgaatttccaaataaaagcgcaattaaatgcctaggatccactgtgcg

SEQ ID NO: 12 - DNA, 7236 bp, Lymphocytic choriomeningitis virus; identical to SEQ ID NO: 2
  misc_feature 1..7236: note = LCMV strain P52-WE
  source 1..7236: mol_type = genomic DNA

SEQ ID NO: 13 - DNA, 1497 bp, Lymphocytic choriomeningitis virus
  misc_feature 1..1497: note = LCMV strain P52-WE
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctatgagccagtgttggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 14 - protein, 498 aa, Lymphocytic choriomeningitis virus
  REGION 1..498: note = misc_feature - LCMV strain P52-WE
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAMSQCWTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 15 - DNA, 1677 bp, Lymphocytic choriomeningitis virus; identical to SEQ ID NO: 5
  misc_feature 1..1677: note = LCMV strain P52-WE
  source 1..1677: mol_type = genomic DNA

SEQ ID NO: 16 - protein, 558 aa, Lymphocytic choriomeningitis virus; identical to SEQ ID NO: 6
  REGION 1..558: note = misc_feature - LCMV strain P52-WE

SEQ ID NO: 17 - DNA, 273 bp, Lymphocytic choriomeningitis virus; identical to SEQ ID NO: 7
  misc_feature 1..273: note = LCMV strain P52-WE
  source 1..273: mol_type = genomic DNA

SEQ ID NO: 18 - protein, 90 aa, Lymphocytic choriomeningitis virus; identical to SEQ ID NO: 8
  REGION 1..90: note = misc_feature - LCMV strain P52-WE

SEQ ID NO: 19 - DNA, 6630 bp, Lymphocytic choriomeningitis virus; identical to SEQ ID NO: 9
  misc_feature 1..6630: note = LCMV strain P52-WE
  source 1..6630: mol_type = genomic DNA

SEQ ID NO: 20 - protein, 2209 aa, Lymphocytic choriomeningitis virus; identical to SEQ ID NO: 10
  REGION 1..2209: note = misc_feature - LCMV strain P52-WE

SEQ ID NO: 21 - DNA, 3376 bp, Lymphocytic choriomeningitis virus
  misc_feature 1..3376: note = LCMV strain Armstrong 53b
  source 1..3376: mol_type = genomic DNA
cgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaataaaaagacctttgaccacacactcatgagtatagtttcgagcctacacctcagtatcagagggaactccaactataaggcagtatcctgcgacttcaacaatggcataaccatccaatacaacttgacattctcaaatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 22 - DNA, 7228 bp, Lymphocytic choriomeningitis virus
  misc_feature 1..7228: note = LCMV strain Armstrong 53b
  source 1..7228: mol_type = genomic DNA
cgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcgcccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcgatgtcctcggccaccgacccgcccagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatataaccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattttgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgagaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagacgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattccactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattatgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

SEQ ID NO: 23 - DNA, 1497 bp, Lymphocytic choriomeningitis virus
  misc_feature 1..1497: note = LCMV strain Armstrong 53b
  source 1..1497: mol_type = genomic DNA
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaataaaaagacctttgaccacacactcatgagtatagtttcgagcctacacctcagtatcagagggaactccaactataaggcagtatcctgcgacttcaacaatggcataaccatccaatacaacttgacattctcaaatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 24 - protein, 498 aa, Lymphocytic choriomeningitis virus
  REGION 1..498: note = misc_feature - LCMV strain Armstrong 53b
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSNAQSAQSQCRTFRGRVLDMFRTAFGGKYMRSGWGWTGSDGKTTWCSQTSYQYLIIQNRTWENHCTYAGPFGMSRILLSQEKTKFFTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 25 - DNA, 1677 bp, Lymphocytic choriomeningitis virus
  misc_feature 1..1677: note = LCMV strain Armstrong 53b
  source 1..1677: mol_type = genomic DNA
atgtccttgtctaaggaagttaagagcttccaatggacgcaagcattgagaagagaattgcagagcttcacatcagatgtgaaggctgctgtcattaaggatgcaaccaaccttctgaatgggttggacttctctgaggtcagcaatgttcagaggatcatgaggaaggaaaagagagatgacaaagacctacagagactcagaagtctcaaccagactgtacattctcttgtggatttaaagtcaacatcaaagaagaatgttttgaaagtggggaggctcagtgcagaagaactgatgtctcttgcggctgaccttgagaagctgaaggccaagatcatgaggtctgaaaggccccaggcttcaggggtatatatggggaacttaacaacacagcaactagaccaaagatctcagatcctacagatagttgggatgagaaagcctcagcagggtgcaagtggtgtggtaagagtttgggatgtgaaagactcatcacttttgaacaatcaatttggcacaatgccaagtctaactatggcttgtatggccaaacagtcacagactccgctcaatgacgttgtacaagcgctcacagaccttggcttgctttacacagtcaagtatccaaatcttaatgatcttgaaaggctgaaagacaagcacccagttctgggggtcatcactgaacagcagtccagcatcaacatttctggctataactttagtcttggtgctgccgtgaaggcaggggcagccctgttggatgggggtaacatgttagagtcaattttgatcaagccaagcaacagcgaggacctcttgaaggcagttctcggggccaagagaaaactcaacatgtttgtttcagaccaagttggggacaggaacccttatgaaaacatcctctataaagtttgcctttcaggtgaaggatggccatacatagcttgtagaacatcgattgtggggagagcatgggaaaacacaacaattgatctcacaagcgagaaacctgcagtcaactcacccaggccagcgcctggagcagcaggtccacctcaggtgggcttaagctacagccagacaatgcttttaaaagacctcatgggaggaattgaccccaacgctcctacatggattgacattgagggtagatttaatgatccagtggaaatagcaattttccaaccacagaacgggcagttcatacacttttacagggaacccgttgatcaaaaacaattcaagcaagattccaagtactcacacggcatggatcttgccgacctcttcaatgcgcaacccgggttgacctcgtcagttataggtgctcttccgcaggggatggttctaagctgtcaaggctccgatgacatcagaaagcttctggactcacagaataggaaggacattaagcttatcgatgttgaaatgaccagggaagcttcgagggagtatgaagacaaagtgtgggacaaatatggctggttgtgtaagatgcatactggaatagtaagggacaaaaagaagaaagagatcaccccgcactgtgcactcatggactgcatcatttttgaaagcgcctccaaagcaaggctcccagatctgaaaactgttcacaacattctgccacatgacctaatttttagaggcccaaatgttgtgacactctaa

SEQ ID NO: 26 - protein, 558 aa, Lymphocytic choriomeningitis virus
  REGION 1..558: note = misc_feature - LCMV strain Armstrong 53b
MSLSKEVKSFQWTQALRRELQSFTSDVKAAVIKDATNLLNGLDFSEVSNVQRIMRKEKRDDKDLQRLRSLNQTVHSLVDLKSTSKKNVLKVGRLSAEELMSLAADLEKLKAKIMRSERPQASGVYMGNLTTQQLDQRSQILQIVGMRKPQQGASGVVRVWDVKDSSLLNNQFGTMPSLTMACMAKQSQTPLNDVVQALTDLGLLYTVKYPNLNDLERLKDKHPVLGVITEQQSSINISGYNFSLGAAVKAGAALLDGGNMLESILIKPSNSEDLLKAVLGAKRKLNMFVSDQVGDRNPYENILYKVCLSGEGWPYIACRTSIVGRAWENTTIDLTSEKPAVNSPRPAPGAAGPPQVGLSYSQTMLLKDLMGGIDPNAPTWIDIEGRFNDPVEIAIFQPQNGQFIHFYREPVDQKQFKQDSKYSHGMDLADLFNAQPGLTSSVIGALPQGMVLSCQGSDDIRKLLDSQNRKDIKLIDVEMTREASREYEDKVWDKYGWLCKMHTGIVRDKKKKEITPHCALMDCIIFESASKARLPDLKTVHNILPHDLIFRGPNVVTL

SEQ ID NO: 27 - DNA, 273 bp, Lymphocytic choriomeningitis virus
  misc_feature 1..273: note = LCMV strain Armstrong 53b
  source 1..273: mol_type = genomic DNA
atgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaa

SEQ ID NO: 28 - protein, 90 aa, Lymphocytic choriomeningitis virus
  REGION 1..90: note = misc_feature - LCMV strain Armstrong 53b
MGQGKSREEKGTNSTNRAEILPDTTYLGPLSCKSCWQKFDSLVRCHDHYLCRHCLNLLLSVSDRCPLCKYPLPTRLKISTAPSSPPPYEE

SEQ ID NO: 29 - DNA, 6633 bp, Lymphocytic choriomeningitis virus
  misc_feature 1..6633: note = LCMV strain Armstrong 53b
  source 1..6633: mol_type = genomic DNA
atggatgaaatcatctcagaattgagagagttatgtttaaactatatagaacaggatgagaggttgtcaaggcagaaactcaactttctgggacaaagggaacccagaatggttctgattgagggactcaagttgctgtcacgctgcattgaaatagacagtgcagacaagagtggctgcacacacaaccacgacgataagtctgtggaaacaattttggtggagtctggaattgtatgcccaggactaccacttatcattcctgatggttacaagctgatagacaattctctcattcttcttgagtgttttgttaggagcactccagccagttttgagaagaaatttatagaggacactaacaaattggcatgcatcagggaagaccttgctgttgcgggtgtcacattagttccaatagtagatggtcgttgtgattatgataatagttttatgccagagtgggcaaacttcaaatttagagaccttttattcaaacttttggagtattctaaccaaaatgagaaagtctttgaagagtctgaatattttagactctgtgagtccctgaagactactatcgacaagcgctccggtatggactctatgaaaattctgaaagatgcgaggtcaactcataatgatgaaattatgaggatgtgccacgaaggcatcaaccccaacatgagctgtgatgatgtggtttttggaataaactctcttttcagcaggtttagaagagatttagaaagtgggaaattaaagagaaactttcagaaagtaaaccctgaaggcttgatcaaggaattctctgagctctatgaaaaccttgctgatagtgatgatatcttaacattaagcagggaggcagtggaatcctgtcctttgatgagattcataactgcagagacccatgggcacgaaaggggaagtgagactagcactgaatatgagaggctcctctctatgttaaacaaagtcaagagtttgaaactgttgaatactagaaggagacagttgttaaatctggatgttttgtgtctttcctcattgataaaacagtcgaaattcaaagggttaaaaaatgataaacactgggtgggttgttgctatagtagtgtgaatgataggctggtaagctttcacagcactaaagaggagttcattagacttttgaggaatagaaaaaagtcaaaggtgtttagaaaggtgtcttttgaggaattgtttagggcgtctattagtgagttcattgcaaaaattcaaaaatgcctgttagtggtgggactgagtttcgagcattacggactgtctgaacaccttgagcaagaatgccacataccattcactgaatttgagaactttatgaaaattggagctcacccgataatgtattatacgaagtttgaagattacaatttccaacccagcacagagcagctgaagaacatacagagcctgagaagattatcatctgtttgtctggccttaacaaacagtatgaaaactagctcagttgctagactaaggcaaaatcaaatagggtctgtgagatatcaagtggtagaatgcaaagaagtgttttgccaagtaataaaactggactctgaagaataccacctattataccagaagactggagaatcttcaaggtgctactccatacaaggcccggatggtcatttaatttccttctatgcagatcctaaaaggttctttttaccaattttttcagatgaggtcttatacaatatgatagacatcatgatttcatggattagatcatgtcctgatttgaaagactgtctcaccgacattgaggttgcactgaggaccctattgttgctaatgctcaccaacccaacaaagagaaatcaaaagcaggtacagagtgtcagatatttggtgatggcaatagtgtcagatttctcatctacatcattaatggataagttgagggaggatctgatcacacctgctgagaaggtggtgtataagctgcttagattcctaataaaaactatttttggtactggtgagaaggtgttgttgagtgcaaaatttaaatttatgttgaatgtgtcatacctgtgtcatttgatcacaaaggagacccctgacaggctaacagatcagataaaatgttttgaaaagttctttgagcccaaaagtcaatttggtttttttgtcaaccccaaggaagcaatcactcctgaggaagaatgtgtgttctatgagcaaatgaagagattcactagtaaagaaattgactgtcagcatacaactccaggtgttaatctggaagcctttagcctaatggtgtcttcatttaacaacggcactttaattttcaaaggagagaagaagctaaacagcctagatcccatgactaactctggatgtgcgacagcattagatcttgctagtaacaaaagtgtggtggttaataagcatctaaatggagaacgacttctggaatatgactttaacaaattgcttgttagtgctgtgagtcaaattacggagagtttcgtaagaaaacaaaagtataagttgagccactcagactatgaatataaagtttccaagttagtctctagattggtcatcggttccaagggagaagagacagggagatcggaagacaacctggcagaaatatgttttgatggagaagaagagacaagcttcttcaaaagtctcgaagaaaaggtcaacaccacaatagcacggtacagaagaggtaggagggccaatgacaaaggagatggagaaaaacttacaaatacaaaaggactacatcatttacagcttattctaacagggaagatggctcacttaagaaaagttatcttgtcagaaatatctttccatttagtagaagactttgacccatcatgtctaaccaatgatgacatgaaatttatctgtgaggctgttgagggttccacagagctgtcacctttgtatttcacctcagtcattaaagatcagtgtggcctcgatgagatggcaaaaaacctttgtagaaagttcttttctgagaatgattggttttcttgcatgaagatgattctgttgcaaatgaatgcaaatgcgtactcagggaaatacaggcatatgcaaaggcaaggcttgaatttcaaatttgactgggacaaactggaagaagacgtgagaatcagtgagagggaaagtaattctgagtcccttagtaaagctctgtcgttgacaaaatgtatgagtgctgctttgaaaaatctgtgcttctactcagaagaatcaccaacatcatacacctcagtaggtcctgactctggaaggctgaaatttgcactatcttataaagagcaggttgggggaaatagagaactctatattggagatttgaggacaaaaatgttcacaaggttaatagaagattattttgagtctttttcaagtttcttttcaggctcctgtttaaacaatgataaggaatttgaaaatgcaatcttgtcaatgactatcaatgtgcgggaagggttcttaaactatagtatggatcacagcaaatggggaccaatgatgtgcccatttttgttcttaatgtttctacaaaatctcaaactaggtgatgaccagtatgtgcgttccgggaaagatcatgttagcactttgttaacttggcacatgcataagcttgtcgaggtcccctttcctgttgtgaatgcaatgatgaaatcatatgtcaagtcgaagctaaaacttctcaggggttcagaaacaactgttactgagagaattttcagacaatattttgaaatggggatagtgccatcccatatatccagccttattgatatggggcagggaatcttgcataatgcttctgacttctatggtttgcttagcgagaggttcatcaactactgcattggtgttatctttggcgaaagaccagaggcttacacatcaagtgatgatcagatcactttatttgataggaggctgagtgacctggttgtaagtgatccggaggaagtccttgtcctgttggaattccaatctcatctgagcggcttgttaaacaaatttatcagcccaaaaagtgtggctgggaggttcgctgcagaatttaaatctagattctatgtatggggggaggaagtccctcttctcacaaagtttgtatctgcagcgctacacaatgtcaagtgtaaagagccacatcaactttgtgaaacaatagatacaattgcagatcaagccatcgcaaatggcgtcccagtctccctagttaatagtatccaaaggagaacactggacctcctaaagtatgccaatttccctttggatccatttctactgaataccaacactgatgtgaaagattggctggatggttctagaggttacagaatacaaagactcattgaggaactgtgtcctaatgaaacaaaggttgtaagaaagcttgtaaggaaactgcatcataagctcaaaaatggtgaatttaatgaagaatttttcttagacctatttaacagagataaaaaggaggccattcttcaattgggagacctcctcggtcttgaagaagatctgaatcagttagcagatgttaactggttgaatttgaatgaaatgttcccattaaggatggttttaagacaaaaggtggtttatccatcagtgatgactttccaagaggaaagaatcccatcattgatcaagacactccagaacaaactttgtagtaaattcacaaggggtgcacagaagctgctgtcagaagcaatcaacaagtcagctttccagagttgtatctcatctggctttataggcctttgcaaaactctaggaagcaggtgtgtgagaaacaaaaatagggaaaatctgtatatcaaaaagctgcttgaggatctaaccacagatgatcatgtgacaagagtttgcaatcgggatggtataacgctgtacatttgtgacaaacagtctcatccagaagcccaccgtgatcatatatgccttttaaggcctcttctttgggactacatttgtatttcattgagcaactcttttgagttgggtgtttgggtcctagcagaaccgaccaaagggaagaataacagtgagaacctaactcttaagcacttaaacccatgtgattatgtagcaagaaagcctgagagctcaaggctactggaggacaaagtgaatttgaaccaagtgattcaatctgtgaggcggttatatcccaagatctttgaggatcagcttcttccatttatgtctgacatgagctcaaaaaacatgaggtggagtcccagaattaaattccttgacctctgtgttttaattgatattaactcagaatccttgtcactcatttctcatgttgttaagtggaaaagggatgaacattacactgttctgttttctgaccttgccaattctcatcagcgatctgactccagtctggttgatgaatttgttgttagcacgagggatgtctgcaagaacttcttaaaacaggtgtattttgaatcatttgttcgagaatttgttgcaacaaccaggacattaggcaatttttcatggttccctcataaagaaatgatgccatctgaagatggtgctgaggcactgggcccctttcaatcatttgtctcaaaggtggtgaacaaaaatgtggagaggcctatgtttaggaatgatttgcagtttggttttgggtggttctcttaccgaatgggagatgttgtgtgtaatgctgccatgttgattaggcagggcctgacaaacccaaaggcatttaaatccttaaaggatctgtgggactacatgctcaactacacaaaaggggtattggagttttcaatttcagtggactttacgcacaatcagaataatactgactgtttaaggaaattttcattgatattcttggttaggtgccaattacagaatccaggtgtggctgaacttttatcatgctctcacctctttaagggtgagatagatagaagaatgttggatgaatgcctccacttactgaggacagactctgtcttcaaggtgaacgatggtgtctttgatatcagatctgaagagtttgaggattacatggaagatcccttgatacttggtgattctcttgagcttgagttgttgggctccaaaagaatactggatgggattagatctattgactttgagagagttggacctgagtgggagcctgtgccactgactgtaaagatgggtgccctttttgaaggaagaaaccttgtccaaaatatcattgtgaagctggagaccaaggacatgaaagtctttctagcaggacttgagggctatgaaaagattagtgatgtccttgggaacctcttcctgcatcgattcagaactggtgaacatttgttgggttcagagataagtgtaatcctccaggaactatgtatagacagatctattctgctgattccactgtcgcttttgccagactggttcgcctttaaggattgcagactttgttttagcaaatctaggagcactttgatgtatgaaacagtggggggcaggtttagactcaaggggaggtcctgcgacgattggctgggcgggtcggtggccgaggacatcgactga

SEQ ID NO: 30 - protein, 2210 aa, Lymphocytic choriomeningitis virus
  REGION 1..2210: note = misc_feature - LCMV strain Armstrong 53b
MDEIISELRELCLNYIEQDERLSRQKLNFLGQREPRMVLIEGLKLLSRCIEIDSADKSGCTHNHDDKSVETILVESGIVCPGLPLIIPDGYKLIDNSLILLECFVRSTPASFEKKFIEDTNKLACIREDLAVAGVTLVPIVDGRCDYDNSFMPEWANFKFRDLLFKLLEYSNQNEKVFEESEYFRLCESLKTTIDKRSGMDSMKILKDARSTHNDEIMRMCHEGINPNMSCDDVVFGINSLFSRFRRDLESGKLKRNFQKVNPEGLIKEFSELYENLADSDDILTLSREAVESCPLMRFITAETHGHERGSETSTEYERLLSMLNKVKSLKLLNTRRRQLLNLDVLCLSSLIKQSKFKGLKNDKHWVGCCYSSVNDRLVSFHSTKEEFIRLLRNRKKSKVFRKVSFEELFRASISEFIAKIQKCLLVVGLSFEHYGLSEHLEQECHIPFTEFENFMKIGAHPIMYYTKFEDYNFQPSTEQLKNIQSLRRLSSVCLALTNSMKTSSVARLRQNQIGSVRYQVVECKEVFCQVIKLDSEEYHLLYQKTGESSRCYSIQGPDGHLISFYADPKRFFLPIFSDEVLYNMIDIMISWIRSCPDLKDCLTDIEVALRTLLLLMLTNPTKRNQKQVQSVRYLVMAIVSDFSSTSLMDKLREDLITPAEKVVYKLLRFLIKTIFGTGEKVLLSAKFKFMLNVSYLCHLITKETPDRLTDQIKCFEKFFEPKSQFGFFVNPKEAITPEEECVFYEQMKRFTSKEIDCQHTTPGVNLEAFSLMVSSFNNGTLIFKGEKKLNSLDPMTNSGCATALDLASNKSVVVNKHLNGERLLEYDFNKLLVSAVSQITESFVRKQKYKLSHSDYEYKVSKLVSRLVIGSKGEETGRSEDNLAEICFDGEEETSFFKSLEEKVNTTIARYRRGRRANDKGDGEKLTNTKGLHHLQLILTGKMAHLRKVILSEISFHLVEDFDPSCLTNDDMKFICEAVEGSTELSPLYFTSVIKDQCGLDEMAKNLCRKFFSENDWFSCMKMILLQMNANAYSGKYRHMQRQGLNFKFDWDKLEEDVRISERESNSESLSKALSLTKCMSAALKNLCFYSEESPTSYTSVGPDSGRLKFALSYKEQVGGNRELYIGDLRTKMFTRLIEDYFESFSSFFSGSCLNNDKEFENAILSMTINVREGFLNYSMDHSKWGPMMCPFLFLMFLQNLKLGDDQYVRSGKDHVSTLLTWHMHKLVEVPFPVVNAMMKSYVKSKLKLLRGSETTVTERIFRQYFEMGIVPSHISSLIDMGQGILHNASDFYGLLSERFINYCIGVIFGERPEAYTSSDDQITLFDRRLSDLVVSDPEEVLVLLEFQSHLSGLLNKFISPKSVAGRFAAEFKSRFYVWGEEVPLLTKFVSAALHNVKCKEPHQLCETIDTIADQAIANGVPVSLVNSIQRRTLDLLKYANFPLDPFLLNTNTDVKDWLDGSRGYRIQRLIEELCPNETKVVRKLVRKLHHKLKNGEFNEEFFLDLFNRDKKEAILQLGDLLGLEEDLNQLADVNWLNLNEMFPLRMVLRQKVVYPSVMTFQEERIPSLIKTLQNKLCSKFTRGAQKLLSEAINKSAFQSCISSGFIGLCKTLGSRCVRNKNRENLYIKKLLEDLTTDDHVTRVCNRDGITLYICDKQSHPEAHRDHICLLRPLLWDYICISLSNSFELGVWVLAEPTKGKNNSENLTLKHLNPCDYVARKPESSRLLEDKVNLNQVIQSVRRLYPKIFEDQLLPFMSDMSSKNMRWSPRIKFLDLCVLIDINSESLSLISHVVKWKRDEHYTVLFSDLANSHQRSDSSLVDEFVVSTRDVCKNFLKQVYFESFVREFVATTRTLGNFSWFPHKEMMPSEDGAEALGPFQSFVSKVVNKNVERPMFRNDLQFGFGWFSYRMGDVVCNAAMLIRQGLTNPKAFKSLKDLWDYMLNYTKGVLEFSISVDFTHNQNNTDCLRKFSLIFLVRCQLQNPGVAELLSCSHLFKGEIDRRMLDECLHLLRTDSVFKVNDGVFDIRSEEFEDYMEDPLILGDSLELELLGSKRILDGIRSIDFERVGPEWEPVPLTVKMGALFEGRNLVQNIIVKLETKDMKVFLAGLEGYEKISDVLGNLFLHRFRTGEHLLGSEISVILQELCIDRSILLIPLSLLPDWFAFKDCRLCFSKSRSTLMYETVGGRFRLKGRSCDDWLGGSVAEDID

SEQ ID NO: 31 - DNA, 3377 bp, Lymphocytic choriomeningitis virus
  misc_feature 1..3377: note = LCMV strain Clone13
  source 1..3377: mol_type = genomic DNA
gcgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcctcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgaccaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 32 - DNA, 7229 bp, Lymphocytic choriomeningitis virus
  misc_feature 1..7229: note = LCMV strain Clone13
  source 1..7229: mol_type = genomic DNA
gcgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcacccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcgatgtcctcggccaccgacccgcccagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatatagccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattgtgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgaaaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagatgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattcgactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattgtgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

SEQ ID NO: 33 - DNA, 1497 bp, Lymphocytic choriomeningitis virus
  misc_feature 1..1497: note = LCMV strain Clone13
  source 1..1497: mol_type = genomic DNA
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcctcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgaccaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 34 - protein, 498 aa, Lymphocytic choriomeningitis virus
  REGION 1..498: note = misc_feature - LCMV strain Clone13
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFLTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 35 - DNA, 1677 bp, Lymphocytic choriomeningitis virus
  misc_feature 1..1677: note = LCMV strain Clone13
  source 1..1677: mol_type = genomic DNA
atgtccttgtctaaggaagttaagagcttccaatggacgcaagcattgagaagagaattgcagagcttcacatcagatgtgaaggctgctgtcattaaggatgcaaccaaccttctgaatgggttggacttctctgaggtcagcaatgttcagaggatcatgaggaaggaaaagagagatgacaaagacctacagagactcagaagtctcaaccagactgtacattctcttgtggatttaaagtcaacatcaaagaagaatgttttgaaagtggggaggctcagtgcagaagaactgatgtctcttgcggctgaccttgagaagctgaaggccaagatcatgaggtctgaaaggccccaggcttcaggggtatatatggggaacttaacaacacagcaactagaccaaagatctcagatcctacagatagttgggatgagaaagcctcagcagggtgcaagtggtgtggtaagagtttgggatgtgaaagactcatcacttttgaacaatcaatttggcacaatgccaagtctaactatggcttgtatggccaaacagtcacagactccgctcaatgacgttgtacaagcgctcacagaccttggcttgctttacacagtcaagtatccaaatcttaatgatcttgaaaggctgaaagacaagcacccagttctgggggtcatcactgaacagcagtccagcatcaacatttctggctataactttagtcttggtgctgccgtgaaggcaggggcagccctgttggatgggggtaacatgttagagtcaattttgatcaagccaagcaacagcgaggacctcttgaaggcagttctcggggccaagagaaaactcaacatgtttgtttcagaccaagttggggacaggaacccttatgaaaacatcctctataaagtttgcctttcaggtgaaggatggccatacatagcttgtagaacatcgattgtggggagagcatgggaaaacacaacaattgatctcacaagcgagaaacctgcagtcaattcacccaggccagcgcctggagcagcaggtccacctcaggtgggcttaagctacagccagacaatgcttttaaaagacctcatgggaggaattgaccccaacgctcctacatggattgacattgagggtagatttaatgatccagtggaaatagcaattttccaaccacagaacgggcagttcatacacttttacagggaacccgttgatcaaaaacaattcaagcaagattccaagtactcacacggcatggatcttgccgacctcttcaatgcgcaacccgggttgacctcgtcagttataggtgctcttccgcaggggatggttctaagctgtcaaggctccgatgacatcagaaagcttctggactcacagaataggaaggacattaagcttatcgatgttgaaatgaccagggaagcttcgagggagtatgaagacaaagtgtgggacaaatatggctggttgtgtaagatgcatactggaatagtaagggacaaaaagaagaaagagatcaccccgcactgtgcactcatggactgcatcatttttgaaagcgcctccaaagcaaggctcccagatctgaaaactgttcacaacattctgccacatgacctaatttttagaggcccaaatgttgtgacactctaa

SEQ ID NO: 36 - protein, 558 aa, Lymphocytic choriomeningitis virus; identical to SEQ ID NO: 26
  REGION 1..558: note = misc_feature - LCMV strain Clone13

SEQ ID NO: 37 - DNA, 273 bp, Lymphocytic choriomeningitis virus; identical to SEQ ID NO: 27
  misc_feature 1..273: note = LCMV strain Clone13
  source 1..273: mol_type = genomic DNA

SEQ ID NO: 38 - protein, 90 aa, Lymphocytic choriomeningitis virus; identical to SEQ ID NO: 28
  REGION 1..90: note = misc_feature - LCMV strain Clone13

SEQ ID NO: 39 - DNA, 6633 bp, Lymphocytic choriomeningitis virus
  misc_feature 1..6633: note = LCMV strain Clone13
  source 1..6633: mol_type = genomic DNA
atggatgaaatcatctcagaattgagagagttatgtttaaactatatagaacaggatgagaggttgtcaaggcagaaactcaactttctgggacaaagggaacccagaatggttctgattgagggactcaagttgctgtcacgctgcattgaaatagacagtgcagacaagagtggctgcacacacaaccacgacgataagtctgtggaaacaattttggtggagtctggaattgtatgcccaggactaccacttatcattcctgatggttacaagctgatagacaattctctcattcttcttgagtgttttgttaggagcactccagccagttttgagaagaaatttatagaggacactaacaaattggcatgcatcagggaagaccttgctgttgcgggtgtcacattagttccaatagtagatggtcgttgtgattatgataatagttttatgccagagtgggcaaacttcaaatttagagaccttttattcaaacttttggagtattctaaccaaaatgagaaagtctttgaagagtctgaatattttagactctgtgagtccctgaagactactatcgacaagcgctccggtatggactctatgaaaattctgaaagatgcgaggtcaactcacaatgatgaaattatgaggatgtgccacgaaggcatcaaccccaacatgagctgtgatgatgtggtttttggaataaactctcttttcagcaggtttagaagagatttagaaagtgggaaattaaagagaaactttcagaaagtaaaccctgaaggcttgatcaaggaattctctgagctctatgaaaaccttgctgatagtgatgatatcttaacattaagcagggaggcagtcgaatcctgtcctttgatgagattcataactgcagagacccatgggcacgaaaggggaagtgagactagcactgaatatgagaggctcctctctatgttaaacaaagtcaagagtttgaaactgttgaatactagaaggagacagttgttaaatctggatgttttgtgtctttcctcattgataaaacagtcgaaattcaaagggttaaaaaatgataaacactgggtgggttgttgctatagtagtgtgaatgataggctggtaagctttcacagcactaaagaggagttcattagacttttgaggaatagaaaaaagtcaaaggtgtttagaaaggtgtcttttgaggaattgtttagggcatctattagtgagttcattgcaaaaattcaaaaatgcctgttagtggtgggactgagtttcgagcattacggactgtctgaacaccttgagcaagaatgccacataccattcactgaatttgagaactttatgaaaattggagctcacccgataatgtattatacgaagtttgaagattacaatttccaacccagcacagagcagctgaagaacatacagagcctgagaagattatcatctgtttgtctggccttaacaaacagtatgaaaactagctcagttgctagactaaggcaaaatcaaatagggtctgtgagatatcaagtggtagaatgcaaagaagtgttttgccaagtaataaaactggactctgaagaataccacctattataccagaagactggagaatcttcaaggtgctactccatacaaggcccggatggtcatttaatttccttctatgcagatcctaaaaggttctttttaccaattttttcagatgaggtcttatacaatatgatagacatcatgatttcatggattagatcatgtcctgatttgaaagactgtctcaccgacattgaggttgcactgaggaccctattgttgctaatgctcaccaacccaacaaagagaaatcaaaagcaggtacagagtgtcagatatttggtgatggcaatagtgtcagatttttcatctacatcattaatggataagttgagggaggatctgatcacacctgctgagaaggtggtgtataagctgcttagattcctaataaaaactatttttggtactggtgagaaggtgttgttgagtgcaaaatttaaatttatgttgaatgtgtcatacctgtgtcatttgatcacaaaggagacccctgacaggctaacagatcagataaaatgttttgaaaagttctttgagcccaaaagtcaatttggtttttttgtcaaccccaaggaagcaatcactcctgaggaagaatgtgtgttctatgagcaaatgaagagattcactagtaaagaaattgactgtcagcatacaactccaggtgttaatctggaagcctttagcctaatggtgtcttcatttaacaacggcactttaattttcaaaggagagaagaagctaaacagcctagatcccatgactaactctggatgtgcgacagcattagatcttgctagtaacaaaagtgtggtggttaataagcatctaaatggagaacgacttctggaatatgactttaacaaattgcttgttagtgctgtgagtcaaattacggagagtttcgtaagaaaacaaaagtataagttgagccactcagactatgaatataaagtttccaagttagtctctagattggtcatcggttccaagggagaagagacagggagatcggaagacaacctggcagaaatatgttttgatggagaagaagagacaagcttcttcaaaagtctcgaagaaaaggtcaacaccacaatagcacggtacagaagaggtaggagggccaatgacaaaggagatggagaaaaacttacaaatacaaaaggactacatcatttacagcttattctaacagggaagatggctcacttaagaaaagttatcttgtcagaaatatctttccatttagtagaagactttgacccatcatgtctaaccaatgatgacatgaaatttatctgtgaggctgttgagggttccacagagctgtcacctttgtatttcacctcagtcattaaagatcagtgtggcctcgatgagatggcaaaaaacctttgtagaaagttcttttctgagaatgattggttttcttgcatgaagatgattctgttgcaaatgaatgcaaatgcgtactcagggaaatacaggcatatgcaaaggcaaggcttgaatttcaaatttgactgggacaaactggaagaagacgtgagaatcagtgagagggaaagtaattctgagtcccttagtaaagctctgtcgttgacacaatgtatgagtgctgctttgaaaaatctgtgcttctactcagaagaatcaccaacatcatacacctcagtaggtcctgactctggaaggctgaaatttgcactatcttataaagagcaggttgggggaaatagagaactctatattggagatttgaggacaaaaatgttcacaaggttaatagaagattattttgagtctttttcaagtttcttttcaggctcctgtttaaacaatgataaggaatttgaaaatgcaatcttgtcaatgactatcaatgtgcgggaagggttcttaaactatagtatggatcacagcaaatggggaccaatgatgtgcccatttttgttcttaatgtttctacaaaatctcaaactaggtgatgaccagtatgtgcgttccgggaaagatcatgttagcactttgttaacttggcacatgcataagcttgtcgaggtcccctttcctgttgtgaatgcaatgatgaaatcatatgtcaagtcgaagctaaaacttctcaggggttcagaaacaactgttactgagagaattttcagacaatattttgaaatggggatagtgccatcccatatatccagccttattgatatggggcagggaatcttgcataatgcttctgacttctatggtttgcttagcgagaggttcatcaactactgcattggtgttatctttggcgaaagaccagaggcttacacatcaagtgatgatcagatcactttatttgataggaggctgagtgacctggttgtaagtgatccggaggaagtccttgtcctgttggaattccaatctcatctgagcggcttgttaaacaaatttatcagcccaaaaagtgtggctgggaggttcgctgcagaatttaaatctagattctatgtatggggggaggaagtccctcttctcacaaagtttgtatctgcagcgctacacaatgtcaagtgtaaagagccacatcaactttgtgaaacaatagatacaattgcagatcaagccatcgcaaatggcgtcccagtctccctagttaatagtatccaaaggagaacactggacctcctaaagtatgccaatttccctttggatccatttctactgaataccaacactgatgtgaaagattggctggatggttctagaggttacagaatacaaagactcattgaggaactgtgtcctaatgaaacaaaggttgtaagaaagcttgtaaggaaactgcatcataagctcaaaaatggtgaatttaatgaagaatttttcttagacctatttaacagagataaaaaggaggccattcttcaattgggagacctcctcggtcttgaagaagatctgaatcagttagcagatgttaactggttgaatttgaatgaaatgttcccattaaggatggttttaagacaaaaggtggtttatccatcagtgatgactttccaagaggaaagaatcccatcattgatcaagacactccagaacaaactttgtagtaaattcacaaggggtgcacagaagctgctgtcagaagcaatcaacaagtcagctttccagagttgtatctcatctggctttataggcctttgcaaaactctaggaagcaggtgtgtgagaaacaaaaatagggaaaatctgtatatcaaaaagctgcttgaggatctaaccacagatgatcatgtgacaagagtttgcaatcgggatggtataacgctgtacatttgtgacaaacagtctcatccagaagcccaccgtgatcatatatgccttttaaggcctcttctttgggactacatttgtatttcattgagcaactcttttgagttgggtgtttgggtcctagcagaaccgaccaaagggaagaataacagtgagaacctaactcttaagcacttaaacccatgtgattatgtagcaagaaagcctgagagctcaaggctactggaggacaaagtgaatttgaaccaagtgattcaatctgtgaggcggctatatcccaagatctttgaggatcagcttcttccatttatgtctgacatgagctcaaaaaacatgaggtggagtcccagaattaaattccttgacctctgtgttttaattgatattaactcagaatccttgtcactcatttctcatgttgttaagtggaaaagggatgaacattacactgttctgttttctgaccttgccaattctcatcagcgatctgactccagtctggttgatgaatttgttgttagcacgagggatgtctgcaagaacttcttaaaacaggtgtattttgaatcatttgttcgagaatttgttgcaacaaccaggacattaggcaatttttcatggttccctcataaagaaatgatgccatctgaagatggtgctgaggcactgggcccctttcaatcatttgtctcaaaggtggtgaacaaaaatgtggagaggcctatgtttaggaatgatttgcagtttggttttgggtggttctcttaccgaatgggagatgttgtgtgtaatgctgccatgttgattaggcagggcctgacaaacccaaaggcatttaaatccttaaaggatctgtgggactacatgctcaactacacaaaaggggtattggagttttcaatttcagtggactttacgcacaatcagaataatactgactgtttaaggaaattttcattgatattcttggttaggtgccaattacagaatccaggtgtggctgaacttttatcatgctctcacctctttaagggtgagatagatagaagaatgttggatgaatgcctccacttactgaggacagactctgtcttcaaggtgaacgatggtgtctttgatatcagatctgaagagtttgaggattacatggaagatcccttgatacttggtgattctcttgagcttgagttgttgggctccaaaagaatactggatgggattagatctattgactttgagagagttggacctgagtgggagcctgtgccactgactgtaaagatgggtgccctttttgaaggaagaaaccttgtccaaaatatcattgtgaagctggagaccaaggacatgaaagtctttctagcaggacttgagggctatgaaaagattagtgatgtccttgggaacctcttcctgcatcgattcagaactggtgaacatttgttgggttcagagataagtgtaatcctccaggaactatgtatagacagatctattctgctgattccactgtcgcttttgccagactggttcgcctttaaggattgcagactttgttttagcaaatctaggagcactttgatgtatgaaacagtggggggcaggtttagactcaaggggaggtcctgcgacgattggctgggcgggtcggtggccgaggacatcgactga

SEQ ID NO: 40 - protein, 2210 aa, Lymphocytic choriomeningitis virus
  REGION 1..2210: note = misc_feature - LCMV strain Clone13
MDEIISELRELCLNYIEQDERLSRQKLNFLGQREPRMVLIEGLKLLSRCIEIDSADKSGCTHNHDDKSVETILVESGIVCPGLPLIIPDGYKLIDNSLILLECFVRSTPASFEKKFIEDTNKLACIREDLAVAGVTLVPIVDGRCDYDNSFMPEWANFKFRDLLFKLLEYSNQNEKVFEESEYFRLCESLKTTIDKRSGMDSMKILKDARSTHNDEIMRMCHEGINPNMSCDDVVFGINSLFSRFRRDLESGKLKRNFQKVNPEGLIKEFSELYENLADSDDILTLSREAVESCPLMRFITAETHGHERGSETSTEYERLLSMLNKVKSLKLLNTRRRQLLNLDVLCLSSLIKQSKFKGLKNDKHWVGCCYSSVNDRLVSFHSTKEEFIRLLRNRKKSKVFRKVSFEELFRASISEFIAKIQKCLLVVGLSFEHYGLSEHLEQECHIPFTEFENFMKIGAHPIMYYTKFEDYNFQPSTEQLKNIQSLRRLSSVCLALTNSMKTSSVARLRQNQIGSVRYQVVECKEVFCQVIKLDSEEYHLLYQKTGESSRCYSIQGPDGHLISFYADPKRFFLPIFSDEVLYNMIDIMISWIRSCPDLKDCLTDIEVALRTLLLLMLTNPTKRNQKQVQSVRYLVMAIVSDFSSTSLMDKLREDLITPAEKVVYKLLRFLIKTIFGTGEKVLLSAKFKFMLNVSYLCHLITKETPDRLTDQIKCFEKFFEPKSQFGFFVNPKEAITPEEECVFYEQMKRFTSKEIDCQHTTPGVNLEAFSLMVSSFNNGTLIFKGEKKLNSLDPMTNSGCATALDLASNKSVVVNKHLNGERLLEYDFNKLLVSAVSQITESFVRKQKYKLSHSDYEYKVSKLVSRLVIGSKGEETGRSEDNLAEICFDGEEETSFFKSLEEKVNTTIARYRRGRRANDKGDGEKLTNTKGLHHLQLILTGKMAHLRKVILSEISFHLVEDFDPSCLTNDDMKFICEAVEGSTELSPLYFTSVIKDQCGLDEMAKNLCRKFFSENDWFSCMKMILLQMNANAYSGKYRHMQRQGLNFKFDWDKLEEDVRISERESNSESLSKALSLTQCMSAALKNLCFYSEESPTSYTSVGPDSGRLKFALSYKEQVGGNRELYIGDLRTKMFTRLIEDYFESFSSFFSGSCLNNDKEFENAILSMTINVREGFLNYSMDHSKWGPMMCPFLFLMFLQNLKLGDDQYVRSGKDHVSTLLTWHMHKLVEVPFPVVNAMMKSYVKSKLKLLRGSETTVTERIFRQYFEMGIVPSHISSLIDMGQGILHNASDFYGLLSERFINYCIGVIFGERPEAYTSSDDQITLFDRRLSDLVVSDPEEVLVLLEFQSHLSGLLNKFISPKSVAGRFAAEFKSRFYVWGEEVPLLTKFVSAALHNVKCKEPHQLCETIDTIADQAIANGVPVSLVNSIQRRTLDLLKYANFPLDPFLLNTNTDVKDWLDGSRGYRIQRLIEELCPNETKVVRKLVRKLHHKLKNGEFNEEFFLDLFNRDKKEAILQLGDLLGLEEDLNQLADVNWLNLNEMFPLRMVLRQKVVYPSVMTFQEERIPSLIKTLQNKLCSKFTRGAQKLLSEAINKSAFQSCISSGFIGLCKTLGSRCVRNKNRENLYIKKLLEDLTTDDHVTRVCNRDGITLYICDKQSHPEAHRDHICLLRPLLWDYICISLSNSFELGVWVLAEPTKGKNNSENLTLKHLNPCDYVARKPESSRLLEDKVNLNQVIQSVRRLYPKIFEDQLLPFMSDMSSKNMRWSPRIKFLDLCVLIDINSESLSLISHVVKWKRDEHYTVLFSDLANSHQRSDSSLVDEFVVSTRDVCKNFLKQVYFESFVREFVATTRTLGNFSWFPHKEMMPSEDGAEALGPFQSFVSKVVNKNVERPMFRNDLQFGFGWFSYRMGDVVCNAAMLIRQGLTNPKAFKSLKDLWDYMLNYTKGVLEFSISVDFTHNQNNTDCLRKFSLIFLVRCQLQNPGVAELLSCSHLFKGEIDRRMLDECLHLLRTDSVFKVNDGVFDIRSEEFEDYMEDPLILGDSLELELLGSKRILDGIRSIDFERVGPEWEPVPLTVKMGALFEGRNLVQNIIVKLETKDMKVFLAGLEGYEKISDVLGNLFLHRFRTGEHLLGSEISVILQELCIDRSILLIPLSLLPDWFAFKDCRLCFSKSRSTLMYETVGGRFRLKGRSCDDWLGGSVAEDID

(19) United States
(12) Patent Application Publication
(10) Pub. No.: US 2026/0345704 A1
(43) Pub. Date: Nov. 5, 2026

(54) VIRUS PARTICLES FOR THERAPEUTIC PURPOSES

(71) Applicant: Abalos Therapeutics GmbH, Dusseldorf (DE)

(72) Inventors: Jorg VOLLMER, Dusseldorf (DE); Marcus KOSTKA, Dusseldorf (DE); Philipp LANG, Dusseldorf (DE); Haifeng XU, Dusseldorf (DE)

(73) Assignee: Abalos Therapeutics GmbH, Dusseldorf (DE)

(21) Appl. No.: 19/743,264

(22) Filed: Jul. 15, 2026

Related U.S. Application Data
(62) Division of application No. 17/680,960, filed on Feb. 25, 2022, now Pat. No. 12,708,654.

(30) Foreign Application Priority Data
Feb. 26, 2021 (EP) 21159746.3

(51) Int. Cl. A61K 35/768; A61P 35/00

(52) U.S. Cl. CPC A61K 35/768; A61P 35/00

(57) ABSTRACT

The invention relates to a virus particle comprising a lymphocytic choriomeningitis virus (LCMV) S segment and an LCMV L segment, wherein the S segment comprises an open reading frame encoding a glycoprotein derived from LCMV strain WE, and wherein the L segment comprises an open reading frame encoding an L protein derived from LCMV strain Clone13. The invention also relates to related host cells, methods of producing such virus particles, pharmaceutical compositions comprising such virus particles, and medical uses of such virus particles.

### CROSS REFERENCE TO RELATED APPLICATIONS

The present application is a divisional of U.S. patent application Ser. No. 17/680,960, filed on Feb. 25, 2022, which claims the benefit of priority of European Patent Application No. 21159746.3 filed on Feb. 26, 2021, the contents of each of which are hereby incorporated by reference in their entirety for all purposes.

### SEQUENCE LISTING

The application contains a Sequence Listing which has been submitted electronically in .XML format and is hereby incorporated by reference in its entirety. Said .XML copy, created on Jun. 269, 2026, is named “SCH-7600-DV1_SeqListing.xml” and is 151,552 bytes in size. The sequence listing contained in this .XML file is part of the specification and is hereby incorporated by reference herein in its entirety.

### FIELD OF THE INVENTION

The invention relates to a virus particle comprising a lymphocytic choriomeningitis virus (LCMV) S segment and an LCMV L segment, wherein the S segment comprises an open reading frame encoding a glycoprotein derived from LCMV strain WE, and wherein the L segment comprises an open reading frame encoding an L protein derived from LCMV strain Clone13. The invention also relates to related host cells, methods of producing such virus particles, pharmaceutical compositions comprising such virus particles, and medical uses of such virus particles.

### BACKGROUND

LCMV belongs to the genus of Mammarenaviruses and can ubiquitously be isolated from mice and other rodents (Radoshitzky et al. (2020) in D. M. Knipe and P. M. Howley (eds.), Fields Virology). Intrauterine infection results in persistent infection of the offspring without clinical symptoms although the viral load may be very high (Radoshitzky et al. (2020) in D. M. Knipe and P. M. Howley (eds.), Fields Virology). Live virus is shed from infected animals lifelong via urine, saliva, nasal secretions and droppings resulting in symptomatic infection of exposed naïve rodents.

Humans become infected with LCMV through close contact with infected rodents or secretions from infected rodents, through solid organ transplantations or by vertical transplacental transmission. Human to human horizontal transmission other than through solid organ transplantation has not been reported. Postnatal infections in persons with an intact immune system are often asymptomatic or result in mild febrile illness. Mild and reversible neurological symptoms are rarely observed. Infection during pregnancy increases the risk for miscarriage, severe CNS or ocular malformations. Neurologic sequelae include spastic quadriparesis, mental retardation, seizures and visual impairment. In immune suppressed organ recipients LCMV infection can be fatal, however, less than 20 cases have been described to date.

LCMV strains have been divided into four different lineages grouping the most commonly used laboratory strains Armstrong and WE into lineage I (Radoshitzky et al. (2020) in D. M. Knipe and P. M. Howley (eds.), Fields Virology). These laboratory strains have a low pathogenic potential for healthy humans but can cause substantial disease and death in susceptible animals. In line with the clinical manifestations caused in susceptible animals by the two strains and derivatives derived thereof the Armstrong strain is categorized as neurotropic in contrast to the WE strain that is categorized as viscerotropic.

The LCMV genome consists of two negative stranded RNA segments named S (mall) and L(arge). The S segment encodes the surface Glycoprotein (GP) and the Nucleocapsid protein (NP) while the L segment encodes the viral polymerase (LP) and the Z protein (Radoshitzky et al. (2020) in D. M. Knipe and P. M. Howley (eds.), Fields Virology).

Certain LCMV WE strain derivatives have been described as effective agents for solid tumor treatment (WO 2016/166285 A1, WO 2020/053324 A1). However, LCMV stain WE has a by default biosafety level (BSL) 3 classification, which would preclude the use of such products, as production and application of such strains under BSL 3 conditions is virtually impossible.

Thus, there is an unmet need to provide viruses with a higher level of attenuation while having comparable or even improved anti-tumoral properties in comparison with LCMV stain WE. It is thus object of the invention to provide additional and/or improved viruses that are useful in therapy.

### SUMMARY OF THE INVENTION

The invention relates to a virus particle comprising a lymphocytic choriomeningitis virus (LCMV) S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40, wherein the glycoprotein comprises at least one mutated amino acid residue in comparison with the glycoprotein sequence set forth in SEQ ID NO: 4.

The invention also relates to a virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40, wherein the L protein comprises at position 1079 corresponding to the linear polypeptide sequence of SEQ ID NO: 40 an amino acid residue other than Lys.

The invention also relates to a virus particle comprising an LCMV S segment and an LCMV L segment, wherein the S segment comprises an open reading frame encoding a glycoprotein having at least 97% sequence identity to SEQ ID NO: 4 and an open reading frame encoding a nucleoprotein having at least 97% sequence identity to SEQ ID NO: 6, and wherein the L segment comprises an open reading frame encoding an L protein having at least 90% sequence identity to SEQ ID NO: 40 and an open reading frame encoding a Z protein having at least 90% sequence identity to SEQ ID NO: 38.

The invention also relates to a host cell comprising an LCMV S segment and an LCMV L segment as comprised in a virus particle of the invention.

The invention also relates to a host cell comprising cDNA of (a) an ORF encoding a glycoprotein, an ORF encoding an L protein, an ORF encoding a nucleoprotein, and an ORF encoding a Z protein as comprised in a virus particle of the invention; and/or (b) an LCMV S segment and an LCMV L segment as comprised in a virus particle of the invention.

The invention also relates to a method of producing a virus particle of the invention comprising cultivating the host cell of the invention under conditions suitable for virus particle formation.

The invention also relates to a pharmaceutical composition comprising a virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40.

The invention also relates to a virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40 for use in therapy, in particular in the treatment of cancer.

### BRIEF DESCRIPTION OF THE DRAWINGS

FIG. 1: Murine B16 tumor-bearing B1/6 mice, LCMV wild type (WE) and recombinant strains with 2×10E4 PFU/animal i.v. at day 0, tumor growth measurement for 7 days and tumor mass at day 7 (n=5 animals/group), one way ANOVA and Dunnett's multiple comparisons tests for statistics.

FIG. 2: Murine B16 tumor-bearing B1/6 mice, LCMV wild type (WE) and recombinant strains with 2×10E4 PFU/animal i.v. at day 0, CD8 T cells by flow cytometry from tumor at day 7 (n=5 animals/group).

FIG. 3: Murine B16 tumor-bearing B1/6 mice, LCMV wild type (WE) and recombinant strains with 2×10E4 PFU/animal i.v. at day 0, virus-specific (GP33) CD8 T cells by flow cytometry from tumor at day 7 (n=5 animals/group).

FIG. 4: Murine MC38 tumor-bearing B1/6 mice, LCMV wild type (WE) and recombinant strains with 2×10E4 PFU/animal i.v. at day 0, tumor growth measurement for 11 days (n=4 animals/group), t-test for statistics.

FIG. 5: Murine MC38 tumor-bearing B1/6 mice, LCMV wild type (WE) and recombinant strains with 2×10E4 PFU/animal i.v. at day 0, tumor infiltrating T cells (CD3⁺) measured via flow cytometry at day 11 (n=4 animals/group).

FIG. 6: Murine MC38 tumor-bearing B1/6 mice, LCMV wild type (WE) and recombinant strains with 2×10E4 PFU/animal i.v. at day 0, tumor infiltrating cytotoxic T cells (CD3⁺CD8⁺) measured via flow cytometry at day 11 (n=4 animals/group).

FIG. 7: Murine MC38 tumor-bearing B1/6 mice, LCMV wild type (WE) and recombinant strains with 2×10E4 PFU/animal i.v. at day 0, tumor immunohistochemistry (IHC) for LCMV (NP protein⁺) CD8 T cells (CD8⁺) at day 11 (n=(3) 4 animals/group).

FIG. 8: In vitro replication for 20 h of wild type (WE, Clone13) and recombinant viruses at MOI=0.1 by flow cytometry (NP protein) in HEK293T and human tumor cell lines H1975 (lung), SkMM (myeloma), Ma-Mel-51 (melanoma), C643 (thyroid) and murine tumor cell lines MC-57 (fibrosarcoma) and B16F10 (melanoma).

FIG. 9: In vitro replication for 24 h or 48 h of wild type (WE) and recombinant WE-Cl13 viruses at MOI=0.1 by colony formation assay on MC57 cells, in human tumor cell lines H1975 (lung), Mamel86a and 51 (melanoma), C643 (thyroid), murine tumor cell lines 511950 (pancreas), Tramp-C2 (prostate), B16.F10 (melanoma), and murine dendritic cell line JAWSII.

FIG. 10: In vitro replication for 48 h of wild type (WE) and recombinant viruses at MOI=0.1 by replication assay in human primary neurons, murine tumor cell line MC-57 (fibrosarcoma), murine MC57G cells, human lung adenocarcinoma cell line A549 and human thyroid carcinoma cell line FTC133, line: initial inoculum.

FIG. 11: Bone marrow-derived dendritic cells.

FIG. 12: Treatment of B1/6 mice with 2×10E4 PFU/animal i.v., serum cytokines at days 1 and 3 p.i. Dotted line indicates lower detection limit.

FIG. 13: Treatment of B1/6 mice with 2×10E4PFU/animal i.v., serum cytokines at days 1 and 3 p.i. Dotted line indicates lower detection limit.

FIG. 14: Mouse strains with LCMV wild type (WE, Clone13, Arm) and recombinant (Clone13 L, WE S) strains with 2×10E6 PFU/animal i.v. at day 0 (n=3 animals/group).

FIG. 15: Mouse strains with LCMV wild type (WE, Clone13, Arm) and recombinant (Clone13 L, WE S) strains with 2×10E6 PFU/animal i.v. at day 0 (n=3 animals/group).

FIG. 16: Mouse strains with LCMV wild type (WE, Clone13, Arm) and recombinant (Clone13 L, WE S) strains with 2×10E6 PFU/animal i.v. at day 0 (n=3 animals/group).

FIG. 17: Mouse strains with LCMV wild type (WE, Clone13, Arm) and recombinant (Clone13 L, WE S) strains with 2×10E6 PFU/animal i.v. at day 0 (n=3 animals/group).

FIG. 18: ALT/AST serum levels upon i.v. treatment of B16 melanoma-bearing B1/6 animals with 2×10E4 PFU/animal 7 days p.i.

FIG. 19: Murine B16 tumor-bearing B1/6 mice, LCMV wild type (WE) and recombinant strains with 2×10E4 PFU/animal i.v. at day 0, colony forming assay from the indicated organs at day 7 (n=5 animals/group).

FIG. 20: Murine MC38 tumor-bearing B1/6 mice, LCMV wild type (WE) and recombinant strains with 2×10E4 PFU/animal i.v. at day 0, plaque-forming assay from the indicated organs at day 11 (n=4 animals/group).

FIG. 21: In vitro replication for 20 h of wild type (WE) and recombinant viruses at MOI=0.1 by flow cytometry (NP protein) in human tumor cell lines H1975 (lung), GIST-T1 (gastrointestinal), Ma-Mel-86a and 51 (melanoma) and murine tumor cell line MC57G (fibrosarcoma). Line: replication of LCMV strain WE

FIG. 22: In vitro replication for 20 h of wild type (WE) and recombinant viruses at MOI=0.1 by flow cytometry (NP protein) in human tumor cell lines H1975 (lung), Gist-T1 (gastrointestinal), Mamel86a and 51 (melanoma) and murine tumor cell line MC57G (fibrosarcoma). Line: replication of LCMV strain WE

FIG. 23: Differentiated human muscle myoblasts, human skeletal muscle myoblasts (SkMM), lung adenocarcinoma cell line A549 and murine fibrosarcoma cell line MC57G were seeded out (100.000 cell/well) and infected with MOI=0.1 of LCMV strains WE, WE-Cl13(L), P52-Cl13(L), I181M-Cl13(L) or R185W-Cl13(L). After 20h, cells were intracellularly stained for LCMV nucleoprotein (NP, clone VL4). The percentage of LCMV infected cells (LCMV NP⁺) was analyzed via flow cytometry. Line: replication of LCMV strain WE

FIG. 24: In vitro replication for 48 h of wild type (WE) and recombinant viruses at MOI=0.1 by replication assay in human primary neurons and as a comparison murine tumor cell line MC57G (fibrosarcoma), line: initial inoculum.

FIG. 25: Murine B16 tumor-bearing B1/6 mice, LCMV wild type (WE) and recombinant strains with 2×10E4 PFU/animal i.v. at day 0, tumor growth measurement for 7 days and tumor mass at day 7 (n=5 animals/group), one way ANOVA and Dunnett's multiple comparisons tests for statistics.

FIG. 26: Murine B16 tumor-bearing B1/6 mice, LCMV wild type (WE) and recombinant strains with 2×10E4 PFU/animal i.v. at day 0, virus-specific (GP33) and CD8 T cells by flow cytometry from dLN and tumor at day 7 (n=5 animals/group).

FIG. 27: Murine B16 tumor-bearing B1/6 mice, LCMV wild type (WE) and recombinant strains with 2×10E4 PFU/animal i.v. at day 0, virus-specific (GP33) and CD8 T cells by flow cytometry from dLN and tumor at day 7 (n=5 animals/group).

FIG. 28: Murine B16 tumor-bearing B1/6 mice, LCMV wild type (WE) and recombinant strains with 2×10E4 PFU/animal i.v. at day 0, virus-specific (GP33) and CD8 T cells by flow cytometry from dLN and tumor at day 7 (n=5 animals/group).

FIG. 29: Murine B16 tumor-bearing B1/6 mice, LCMV wild type (WE) and recombinant strains with 2×10E4 PFU/animal i.v. at day 0, virus-specific (GP33) and CD8 T cells by flow cytometry from dLN and tumor at day 7 (n=5 animals/group).

FIG. 30: Murine MC38 tumor-bearing B1/6 mice, LCMV wild type (WE) and recombinant strains with 2×10E4 PFU/animal i.v. at day 0, tumor growth measurement for 11 days (n=4 animals/group), t-test for statistics.

FIG. 31: Murine MC38 tumor-bearing B1/6 mice, LCMV wild type (WE) and recombinant strains with 2×10E4 PFU/animal i.v. at day 0, tumor T cell measurement by flow cytometry at day 11, n=4 animals/arm, except for the P52-FP7-Cl13(L) group (n=3) due to experimental error (values near/below control).

FIG. 32: Murine MC38 tumor-bearing B1/6 mice, LCMV wild type (WE) and recombinant strains with 2×10E4 PFU/animal i.v. at day 0, tumor T helper cell measurement by flow cytometry at day 11, n=4 animals/arm, except for the P52-FP7-Cl13(L) group (n=3) due to experimental error (values near/below control).

FIG. 33: Murine MC38 tumor-bearing B1/6 mice, LCMV wild type (WE) and recombinant strains with 2×10E4 PFU/animal i.v. at day 0, tumor cytotoxic T cell measurement by flow cytometry at day 11, n=4 animals/arm, except for the P52-FP7-Cl13(L) group (n=3) due to experimental error (values near/below control).

FIG. 34: Naïve B1/6 mice, LCMV wild type (WE) and recombinant strains with 2×10E4 PFU/animal i.v. at day 0, cytokine LegendPlex days 2 and 4 (n=3 animals/group).

FIG. 35: Murine B16 tumor-bearing B1/6 mice, LCMV wild type (WE) and recombinant strains with 2×10E4 PFU/animal i.v. at day 0, plaque-forming assay from the indicated organs at day 7 (n=5 animals/group).

FIG. 36: Murine MC38 tumor-bearing B1/6 mice, LCMV wild type (WE) and recombinant strains with 2×10E4 PFU/animal i.v. at day 0, plaque-forming assay from the indicated organs at day 11 (n=4 animals/group).

FIG. 37: FreeStyle 293-F suspension cells were infected with MOI=0.001 of LCMV strains WE-Cl13(L), P52-Cl13(L) or P52-WE(L) at a density of 1.5×10⁶ cells per mL. Supernatant was harvested on 12, 24, 30, 34, 40, 48, 72 and 96 hours post infection. LCMV titers in supernatant were analyzed via plaque-forming assay.

FIG. 38: Nucleic acid and amino acid sequences of the L segment, S segment, GPC, NP, ZP, and LP of LCMV strains A. WE, B. P52, C. Armstrong, and D. Clone13.

### DETAILED DESCRIPTION

The inventors of the present application believe that pathogenicity of the WE strain may be consistently determined by elements located on the L segment, while the S segment of the WE and other LCMV strains is not contributing to pathogenicity. As such, replacement of the L segment of the WE strain by the corresponding segment of another LCMV strain, such as the Armstrong strain, Clone13 strain, and derivatives thereof, are believed to result in a reassorted laboratory strain that does no longer display the original pathogenic profile including liver pathogenicity in NHPs and other susceptible animals.

The inventors of the present application have then surprisingly found that a virus strain comprising elements of the S segment of the LCMV WE strain or derivatives thereof, which are believed not to contribute to pathogenicity, and elements of the L segment of the strain Clone13 or Armstrong or derivatives thereof, which are believed not to contribute to pathogenicity, have improved properties as compared to LCMV WE strain, in particular in tumor treatment. Such a virus strain is believed to no longer being able to induce LCMV specific disease including liver disease in NHPs, rodents or guinea pigs (Riviere et al. (1985) Journal of virology, 55:704-09; Riviere et al. (1986) Med Microbiol Immunol, 175:191-2; Baccala et al. (2014) Proc Natl Acad Sci USA, 111:8925-30; Oldstone et al. (2018) Proceedings of the National Academy of Sciences, 115: E7814).

Studies conducted by the inventors of the present application with naïve and susceptible rodents do indeed demonstrate that a reassorted virus according to the invention may have decreased pathogenic properties as compared to LCMV WE strain including liver pathogenicity (Examples 16, 17, 18, 19, and 35). These results do confirm that a virus particle of the invention may result in virus-based products that can safely be handled during manufacturing and clinical investigation as pathogenic properties described for wild-type LCMV strains are reduced, including liver pathogenesis and neurotropism (Examples 10, 21, 24).

Beneath its severely reduced replication capacity (up to >100 times) in cells of different origin (Example 8 and 9), virus particles of the invention may demonstrate a strongly reduced potential to induce in vivo pathogenic cytokines in naïve animals, and at the same time any signs of induced organ pathogenicity may be absent (Examples 12-17). Notably, only virus particles comprising WE strain derived elements of the S segment and Clone13 strain derived elements of the L segment, but not virus particles comprising Clone13 strain derived elements of the S segment and WE strain derived elements of the L segment showed attenuated replication in Example 8, supporting the hypothesis that pathogenicity of the WE and Clone13 strain is determined by its L or S segment, respectively.

The FVB mouse strain, which exhibits a predisposition to viral induced pathologies (Schnell et al. (2012) PLoS Pathog, 8: e1003073), succumbs to a hemorrhagic fever-like illness including thrombocytopenia and hepatocellular necrosis when infected with LCMV. Importantly, the disease in FVB mice mimics LCMV disease in macaques and clinical signs of Argentine hemorrhagic fever (Schnell et al. (2012) PLoS Pathog, 8: e1003073). However, in contrast to LCMV strains WE, Clone13, and Armstrong, infection with a virus particle of the invention did not result in neither one reduced animal weight nor liver pathology or any other signs of hemorrhagic fever-like illness in Examples 14-17.

An important and surprising characteristic of the virus particles of the invention for clinical development is their retainment or even enhancement of strong anti-tumoral effects as described for the WE strain (Kalkavan et al. (2017) Nat Commun, 8:14447)—despite their attenuation. A virus particle of the invention may be equipped with additional modifications to increase its tumor tropism (Examples 21-23). Such modifications may comprise mutations at positions 181 and/or 185 of the GP, in particular Arg 185→Trp and/or Ile 181→Met. Indeed, such virus particles may exhibit strong anti-tumoral effects in murine tumor models (Example 25) but may bear the attenuated phenotype of the WE-Clone13 strain from which it is derived (Examples 34 and 38). Pathogenicity of LCMV was reported as being mainly directed by the anti-viral functionality of the immune system and correlates with the strength of virus replication in infected organs (Lang et al. (2010) Cell Physiol Biochem, 26:263-72). Such modified virus particles may result in a, compared to the WE strain, stronger early LCMV directed T cell response, the main anti-viral immune effector mechanism, and its organ distribution is controlled already early upon infection (Example 35).

In previous publications, LCMV was described as being able to induce organ damage (e.g., liver) due to an overshooting and deregulated innate (Th1 and pro-inflammatory cytokines) and adaptive (CD8 T cells) immune response (Lang et al. (2010) Cell Physiol Biochem, 26:263-72; Schnell et al. (2012) PLoS Pathog, 8: e1003073; Oldstone et al. (2018) Proceedings of the National Academy of Sciences, 115: E7814). The inventors of the present application have surprisingly found that—although a similar pattern of the cytokine response to the wild type LCMV strains Armstrong, Clone13 and WE was observed—the strength of the immune response for a virus particle of the invention may be substantially lower. This observation is especially important for IFN-alpha, for which high cytokine levels early upon infection was linked to organ damage in respective animal models (Schnell et al. (2012) PLoS Pathog, 8: e1003073). The virus particles of the invention, in contrast, may induce especially early upon infection substantially lower IFN-alpha and IFN-gamma levels (Examples 12 and 13), which strongly points to a significantly lower potential for IFN-mediated detrimental effects (Baccala et al. (2014) Proc Natl Acad Sci USA, 111:8925-30). In addition, the pro-inflammatory cytokines IL-6 and TNF-alpha, as well as IL-10 may also be substantially decreased at different time points (days 1 and 3) upon infection, indicating enhanced virus control (Lang et al. (2010) Cell Physiol Biochem, 26:263-72; Schnell et al. (2012) PLoS Pathog, 8: e1003073).

Overall, the virus particles of the invention may exhibit a strongly attenuated phenotype and may show an even stronger in vivo attenuated immune phenotype, although retaining or even enhancing their anti-tumoral efficacy.

Accordingly, the present invention relates to a virus particle comprising an LCMV S segment and an LCMV L segment. The S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40.

The S segment may also comprise an open reading frame encoding a nucleoprotein. The nucleoprotein may have at least 97% sequence identity to SEQ ID NO: 6.

The L segment may also comprise an L segment. The L segment may comprise an open reading frame encoding a Z protein having at least 90% sequence identity to SEQ ID NO: 38.

The virus particle of the invention is preferably an arenavirus particle, more preferably a lymphocytic choriomeningitis virus (LCMV) particle.

The wild-type arenavirus genomic segments and ORFs are known in the art. In particular, the arenavirus genome consists of an S segment and an L segment. The S segment carries the ORFs encoding the GP and the NP. The L segment encodes the L protein and the Z protein. Both segments are flanked by the respective 5′ and 3′ UTRs.

The virus particles of the invention preferably comprise genomic segments that correspond to the genomic segments of a wild-type arenavirus. This means that the S segment carries the ORFs encoding the GP and the NP and that the L segment encodes the L protein and the Z protein. On the S segment, the ORF encoding the glycoprotein is under control of a 5′ untranslated region (UTR) and the nucleoprotein is under control of a 3′ UTR. On the L segment, the ORF encoding the L protein is under control of a 3′ UTR and the ORF encoding the Z protein is under control of a 5′ UTR. Accordingly, the genes of the virus particles of the disclosure are preferably located at their natural positions. The virus particle of the invention thus preferably has a bi-segmented genome. The genome of the virus particle of the invention preferably consists of one L segment and one S segment as described herein. Illustrative examples of LCMV S segments are shown in SEQ ID NOs: 1, 11, 21, and 31. Illustrative examples of LCMV L segments are shown in SEQ ID NOs: 2, 12, 22, and 32.

Several strains of LCMV are part of the present disclosure. As used herein, LCMV “WE”, “strain WE”, “WE strain”, or the like refers to an LCMV having the genomic segments as shown in SEQ ID NOs: 1 and 2. As used herein, LCMV “P52”, “P52-WE”, “strain P52”, “P52 strain”, or the like refers to a variant/derivative of strain WE, which has the genomic segments as shown in SEQ ID NOs: 11 and 12. As used herein, LCMV “Armstrong”, “strain Armstrong”, “Armstrong strain”, or the like refers to LCMV strain Armstrong 53b, which has the genomic segments as shown in SEQ ID NOs: 21 and 22. As used herein, LCMV “Clone13”, “Cl13” “strain Clone13”, “Clone13 strain”, or the like refers to an LCMV which has the genomic segments as shown in SEQ ID NOs: 31 and 32.

The terms “glycoprotein”, “GP”, and “G protein”, which are used interchangeably, refer to an LCMV-derived glycoprotein, which is considered to mediate receptor binding and membrane fusion. Illustrative examples of glycoproteins are shown in SEQ ID NOs: 4, 14, 24, and 34. Illustrative examples for genes that encode a glycoprotein are shown in SEQ ID NOs: 3, 13, 23, and 33.

The terms “L protein”, and “LP”, which are used interchangeably, refer to an LCMV-derived RNA polymerase L. Illustrative examples of L proteins are shown in SEQ ID NOs: 10, 20, 30, and 40. Illustrative examples for genes that encode an L protein are shown in SEQ ID NOs: 9, 19, 29, and 39.

The terms “nucleoprotein”, “N protein”, and “NP”, which are used interchangeably, refer to an LCMV-derived nucleoprotein. Illustrative examples of nucleoproteins are shown in SEQ ID NOs: 6, 16, 26, and 36. Illustrative examples for genes that encode a nucleoprotein are shown in SEQ ID NOs: 5, 15, 25, and 35.

The terms “Z protein” or “ZP”, which are used interchangeably, refer to an LCMV-derived small RING finger protein Z. Illustrative examples of Z proteins are shown in SEQ ID NOs: 8, 18, 28, and 38. Illustrative examples for genes that encode a Z protein are shown in SEQ ID NOs: 7, 17, 27, and 37.

A virus particle of the invention comprises a glycoprotein, wherein the glycoprotein may comprise at least one mutated amino acid residue in comparison with the glycoprotein sequence set forth in SEQ ID NO: 4. For example, the glycoprotein may comprise at least one mutated amino acid residue at position 181 and/or 185 in comparison with the glycoprotein sequence set forth in SEQ ID NO: 4. It is however preferred that the glycoprotein comprises a mutation at both positions in comparison with SEQ ID NO: 4. Preferred mutations at these positions are selected from Arg 185→Trp and Ile 181→Met. A virus particle thus can have any one or preferably both mutations.

Without wishing to be bound by theory, it is believed that the presence of the one or two above-mentioned mutations in the glycoprotein can improve the function of the LCMV (e.g., anti-tumoral activities), independent from the presence of other mutations, in particular of other mutations in other genes or proteins of LCMV.

A virus particle of the invention comprises an L protein, wherein the L protein may comprise at position 1079 corresponding to the linear polypeptide sequence of SEQ ID NO: 40 an amino acid residue other than Lys. The amino acid residue at this position is preferably a non-basic amino acid residue, more preferably a neutral hydrophilic amino acid residue, even more preferably an Asn or Gln, most preferably a Gln.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. Naturally occurring residues are divided into groups based on common side-chain properties: (1) hydrophobic: methionine, alanine, valine, leucine, iso-leucine; (2) neutral hydrophilic: cysteine, serine, threonine, asparagine, glutamine; (3) acidic: aspartic acid, glutamic acid; (4) basic: histidine, lysine, arginine; (5) residues that influence chain orientation: glycine, proline; and (6) aromatic: tryptophan, tyrosine, phenylalanine. In some embodiments, substitutions may entail exchanging a member of one of these classes for another class.

A virus particle of the invention comprises an S segment and an L segment, wherein the S segment is preferably derived from LCMV strain WE or a variant thereof and wherein the L segment is preferably derived from LCMV strain Clone13 or a variant thereof.

A variant of the L segment may have at least about 83%, preferably at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, preferably at least about 99.7%, preferably at least about 99.8%, preferably at least about 99.9% sequence identity to the sequence of the original L segment.

A virus particle disclosed herein may have an L segment that comprise or preferably consists of a sequence that has at least about 83%, preferably at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, preferably at least about 99.7%, preferably at least about 99.8%, preferably at least about 99.9% sequence identity, or is preferably identical, to a sequence set forth in SEQ ID NO: 21 or 31.

A variant of the S segment may have at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, preferably at least about 99.7%, preferably at least about 99.8%, preferably at least about 99.9% sequence identity to the sequence of the original S segment.

A virus particle disclosed herein may have an S segment that comprise or preferably consists of a sequence that has at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, preferably at least about 99.7%, preferably at least about 99.8%, preferably at least about 99.9% sequence identity, or is preferably identical, to a sequence set forth in SEQ ID NO: 1 or 11.

A virus particle of the invention comprising an LCMV preferably comprises S segment and an LCMV L segment, wherein the S segment comprises an open reading frame encoding a glycoprotein having at least 97% sequence identity to SEQ ID NO: 4 and an open reading frame encoding a nucleoprotein having at least 97% sequence identity to SEQ ID NO: 6, and wherein the L segment comprises an open reading frame encoding an L protein having at least 90% sequence identity to SEQ ID NO: 40 and an open reading frame encoding a Z protein having at least 90% sequence identity to SEQ ID NO: 38.

An open reading frame encoding a glycoprotein disclosed herein may encode a sequences that has at least about 98%, preferably at least about 98.5%, preferably at least about 99%, preferably at least about 99.1%, preferably at least about 99.2%, preferably at least about 99.3%, preferably at least about 99.4%, preferably at least about 99.5%, preferably at least about 99.5%, preferably at least about 99.7% sequence identity, or is preferably identical, to a sequence set forth in SEQ ID NO: 4 or 14.

An open reading frame encoding a glycoprotein disclosed herein may comprise a sequence that has at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, or is preferably identical, to a sequence set forth in SEQ ID NO: 3 or 13.

An open reading frame encoding an L protein disclosed herein may encode a sequences that has at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, or is preferably identical, to a sequence set forth in SEQ ID NO: 30 or 40.

An open reading frame encoding an L protein disclosed herein may have at least about 83%, preferably at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, preferably at least about 99.8%, preferably at least about 99.9% sequence identity, or is preferably identical, to a sequence set forth in SEQ ID NO: 29 or 39.

An open reading frame encoding a nucleoprotein disclosed herein may encode a sequence that has at least about 98%, preferably at least about 98.5%, preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is preferably identical, to a sequence set forth in any one of in SEQ ID NO: 6 or 16.

An open reading frame encoding a nucleoprotein disclosed herein may have at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is preferably identical, to a sequence set forth in SEQ ID NO: 5 or 15.

An open reading frame encoding a Z protein disclosed herein may encode a sequence that has at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is preferably identical, to a sequence set forth in SEQ ID NO: 28 or 38.

An open reading frame encoding a Z protein disclosed herein may have at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is identical, to a sequence set forth in SEQ ID NO: 27 or 37.

A preferred virus particle comprises an LCMV S segment and an LCMV L segment, wherein the S segment comprises an open reading frame encoding a glycoprotein having at least 98.5% sequence identity to SEQ ID NO: 4 or 14, with SEQ ID NO: 14 being preferred, and an open reading frame encoding a nucleoprotein having at least 98.5% sequence identity to SEQ ID NO: 6 or 16, with SEQ ID NO: 16 being preferred, and wherein the L segment comprises an open reading frame encoding an L protein having at least 95% sequence identity to SEQ ID NO: 30 or 40, with SEQ ID NO: 40 being preferred, and an open reading frame encoding a Z protein having at least 95% sequence identity to SEQ ID NO: 28 or 38, with SEQ ID NO: 38 being preferred.

A preferred virus particle comprises an LCMV S segment and an LCMV L segment, wherein the S segment comprises an open reading frame encoding a glycoprotein having at least 98% sequence identity to SEQ ID NO: 4 or 14, with SEQ ID NO: 14 being preferred, and an open reading frame encoding a nucleoprotein having at least 98% sequence identity to SEQ ID NO: 6 or 16, with SEQ ID NO: 16 being preferred, and wherein the L segment comprises an open reading frame encoding an L protein having at least 98% sequence identity to SEQ ID NO: 30 or 40, with SEQ ID NO: 40 being preferred, and an open reading frame encoding a Z protein having at least 98% sequence identity to SEQ ID NO: 28 or 38, with SEQ ID NO: 38 being preferred.

A preferred virus particle comprises an LCMV S segment and an LCMV L segment, wherein the S segment comprises an open reading frame encoding a glycoprotein having at least 99% sequence identity to SEQ ID NO: 4 or 14, with SEQ ID NO: 14 being preferred, and an open reading frame encoding a nucleoprotein having at least 99% sequence identity to SEQ ID NO: 6 or 16, with SEQ ID NO: 16 being preferred, and wherein the L segment comprises an open reading frame encoding an L protein having at least 99% sequence identity to SEQ ID NO: 30 or 40, with SEQ ID NO: 40 being preferred, and an open reading frame encoding a Z protein having at least 99% sequence identity to SEQ ID NO: 28 or 38, with SEQ ID NO: 38 being preferred.

A preferred virus particle comprises an LCMV S segment and an LCMV L segment, wherein the S segment comprises an open reading frame encoding a glycoprotein having a sequence set forth in SEQ ID NO: 4 or 14, with SEQ ID NO: 14 being preferred, and an open reading frame encoding a nucleoprotein having a sequence set forth in SEQ ID NO: 6 or 16, with SEQ ID NO: 16 being preferred, and wherein the L segment comprises an open reading frame encoding an L protein having a sequence set forth in SEQ ID NO: 30 or 40, with SEQ ID NO: 40 being preferred, and an open reading frame encoding a Z protein having a sequence set forth in SEQ ID NO: 28 or 38, with SEQ ID NO: 38 being preferred.

A preferred virus particle comprises an LCMV S segment and an LCMV L segment, wherein the S segment comprises an open reading frame encoding a glycoprotein having a sequence set forth in SEQ ID NO: 4, and an open reading frame encoding a nucleoprotein having a sequence set forth in SEQ ID NO: 6, and wherein the L segment comprises an open reading frame encoding an L protein having a sequence set forth in SEQ ID NO: 40, and an open reading frame encoding a Z protein having a sequence set forth in SEQ ID NO: 38.

A preferred virus particle comprises an LCMV S segment and an LCMV L segment, wherein the S segment comprises an open reading frame encoding a glycoprotein having a sequence set forth in SEQ ID NO: 14, and an open reading frame encoding a nucleoprotein having a sequence set forth in SEQ ID NO: 16, and wherein the L segment comprises an open reading frame encoding an L protein having a sequence set forth in SEQ ID NO: 40, and an open reading frame encoding a Z protein having a sequence set forth in SEQ ID NO: 38.

A preferred virus particle comprises an LCMV S segment and an LCMV L segment, wherein the S segment comprises an open reading frame encoding a glycoprotein having a sequence set forth in SEQ ID NO: 4, and an open reading frame encoding a nucleoprotein having a sequence set forth in SEQ ID NO: 6, and wherein the L segment comprises an open reading frame encoding an L protein having a sequence set forth in SEQ ID NO: 30, and an open reading frame encoding a Z protein having a sequence set forth in SEQ ID NO: 28.

A preferred virus particle comprises an LCMV S segment and an LCMV L segment, wherein the S segment comprises an open reading frame encoding a glycoprotein having a sequence set forth in SEQ ID NO: 14, and an open reading frame encoding a nucleoprotein having a sequence set forth in SEQ ID NO: 16, and wherein the L segment comprises an open reading frame encoding an L protein having a sequence set forth in SEQ ID NO: 30, and an open reading frame encoding a Z protein having a sequence set forth in SEQ ID NO: 28.

A virus particle of the disclosure preferably does not comprise a heterologous ORF. A “heterologous ORF” in this context refers to an ORF from an organism other than an LCMV and/or a ORF encoding an artificial or synthetic protein.

The term “nucleic acid” as used herein may generally refer to DNA or RNA. DNA and RNA differ—among others—in their nucleobases. The complementary base to adenine in DNA is thymine, whereas in RNA, it is uracil. For the sake of simplification, the corresponding base to adenine is denoted as “t” throughout the application, which—depending on its context—may refer to thymine (in DNA) or uracil (in RNA).

The term “encoding” or “encode(s)” as used herein in the context of a nucleic acid, relates to a nucleic acid having a sequence that can be translated into a particular amino acid sequence that is encoded by the nucleic acid. The nucleic acid sequence may encompass the sequence of a coding strand and may also encompass the sequence of a strand that is complementary to the coding strand.

“Percent (%) sequence identity” with respect to sequences disclosed herein is defined as the percentage of amino acid residues or nucleotides in a candidate sequence that are pair-wise identical with the amino acid residues or nucleotides in a reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is true for nucleotide sequences disclosed herein. For determining sequence identity, uracil (e.g. in RNA) may be considered to be identical to thymine (e.g. in DNA).

The term “attenuation” as used herein, relates to a reduced capacity of a virus to replicate within a given host (cell), a reduced capacity of replicating in a healthy organ, and/or a reduced capacity of inducing cytokines. A virus particle of the invention is preferably attenuated.

The term “infectious” as used herein, relates to a virus' capacity to infect, i.e., enter, a given host cell. A virus particle of the invention is preferably infectious.

The term “replication competent” means that a virus has the ability to amplify and express its genetic material in infected cells and being able to produce further progeny in normal cells that are not genetically engineered. In particular, replication competent viruses do not require host cells that were engineered to express a viral gene for being capable of replication. A virus particle of the invention is preferably replication competent.

The term “pathogenic” as used herein, relates to a virus' capacity to cause disease, i.e., harm to an organism.

A virus particle of the invention may be capable of promoting a reduction of growth of a tumor that is at least as strong or even stronger as compared to LCMV strain WE and/or a virus particle comprising the same S segment as the virus particle and an L segment as shown in SEQ ID NO: 2. In particular, the virus particle may be capable of promoting a stronger reduction of growth of a cold tumor as compared to LCMV strain WE and/or a virus particle comprising the same S segment as the virus particle and an L segment as shown in SEQ ID NO: 2, preferably as measured in an assay as essentially described in Example 1.

A virus particle of the invention may be capable of inducing an adaptive immune activation that is at least as strong or even stronger as compared to LCMV strain WE and/or a virus particle comprising the same S segment as the virus particle and an L segment as shown in SEQ ID NO: 2, preferably as measured in an assay as essentially described in Example 3.

A virus particle of the invention may be capable of promoting a reduction of growth of a warm tumor that is at least as strong as compared to LCMV strain WE and/or a virus particle comprising the same S segment as the virus particle and an L segment as shown in SEQ ID NO: 2, preferably as measured in an assay as essentially described in Example 4.

As used herein a “hot” or “warm” tumor, which is used interchangeably, relates to a tumor having a T-cell-inflamed phenotype. Such tumors show signs of inflammation, meaning the tumor has already been infiltrated by T cells to fight the cancerous cells. Examples of cancers that are typically hot tumors include melanoma, bladder cancer, kidney cancer, head and neck cancer, and non-small cell lung cancer.

As used herein, a “cold tumor” relates to a tumor having a non-T-cell-inflamed phenotype, which has low T cell infiltration or which have not been infiltrated with T cells. Due to the lack of T cells, it difficult to provoke an immune response with immunotherapy drugs in such tumors. Examples of cancers that are typically cold tumors include breast cancer, ovarian cancer, prostate cancer, pancreatic cancer, and glioblastoma. The difference between hot tumors and cold tumors are described in detail in Gajewski et al. (2017) Adv Exp Med Biol. 1036:19-31 and Maleki Vareki (2018) Journal for ImmunoTherapy of Cancer 6:157.

A virus particle of the invention may be capable of inducing decreased levels of an interferon upon (preferably systemic) administration compared to LCMV strain WE, Armstrong, Clone13 and/or a virus particle comprising the same S segment as the virus particle and an L segment as shown in SEQ ID NO: 2, preferably as measured in an assay as essentially described in Example 12. The interferon is preferably selected from the group consisting of IFN-α and IFN-β.

A virus particle of the invention may be capable of inducing a lower concentration of one or more Th1- and pro-inflammatory cytokines compared to LCMV strain WE, Armstrong, Clone13 and/or a virus particle comprising the same S segment as the virus particle and an L segment as shown in SEQ ID NO: 2, preferably as measured in an assay as essentially described in Example 13. The cytokine is preferably selected from the group consisting of IL-6, IL-10, TNFα, and IFNγ.

A virus particle of the invention may be capable of inducing a lower concentration of one or more Th1- and pro-inflammatory cytokines compared to LCMV strain WE, Armstrong, Clone13 and/or a virus particle comprising the same S segment as the virus particle and an L segment as shown in SEQ ID NO: 2, preferably as measured in an assay as essentially described in Example 13. The cytokine is preferably selected from the group consisting of IL-6, IL-10, TNFα, and IFNγ.

A virus particle of the invention may be less pathogenic in a mouse compared to LCMV strain WE, preferably as measured in an FVB/N mouse in an assay as essentially described in Example 14.

A virus particle of the invention may be capable of inducing a reduced degree of liver pathology in a mouse compared to LCMV strain WE and/or a virus particle comprising the same S segment as the virus particle and an L segment as shown in SEQ ID NO: 2, preferably as measured in an assay as essentially described in Example 18.

A virus particle of the invention may be capable of inducing a reduced degree of thrombocytopenia in a mouse compared to LCMV strain WE and/or a virus particle comprising the same S segment as the virus particle and an L segment as shown in SEQ ID NO: 2 or no detectable thrombocytopenia in a mouse, such as an FVB/N mouse, preferably as measured in an assay as essentially described in Example 15.

A virus particle of the invention may show reduced replication in healthy cells such as neuron cells compared to LCMV strain WE and/or a virus particle comprising the same S segment as the virus particle and an L segment as shown in SEQ ID NO: 2, preferably as measured in an assay as essentially described in Example 24.

A virus particle of the invention may show increased replication in tumor cells compared to LCMV strain WE, preferably as measured in an assay as essentially described in Example 24.

A virus particle of the invention may show reduced replication in a healthy organ compared to LCMV strain WE and/or a virus particle comprising the same S segment as the virus particle and an L segment as shown in SEQ ID NO: 2, preferably as measured in an assay as essentially described in Example 35.

The present invention also relates to a host cell comprising an LCMV S segment and an LCMV L segment as described for the virus particle of the invention.

The present invention also relates to a host cell comprising cDNA of an ORF encoding a glycoprotein as described for the virus particle of the invention, an ORF encoding an L protein as described for the virus particle of the invention, an ORF encoding a nucleoprotein as described for the virus particle of the invention, and an ORF encoding a Z protein as described for the virus particle of the invention.

The present invention also relates to a host cell comprising cDNA of an LCMV S segment and an LCMV L segment as described for the virus particle of the invention.

Techniques for the production of a cDNA are routine and conventional techniques of molecular biology and DNA manipulation and production. Any cloning technique known to the skilled artesian can be used. Such as techniques are well known and are available to the skilled artesian in laboratory manuals such as, Sambrook and Russell, Molecular Cloning: A laboratory Manual, 3rd edition, Cold Spring Harbor Laboratory N.Y. (2001).

A cDNA described herein can be incorporated into a plasmid. The cDNA described herein can be part of or can be incorporated into a DNA expression vector and optionally introduced into a host cell. A cDNA described herein or plasmid or vector comprising the cDNA preferably comprises a promoter. Specific examples of promoters include an RNA polymerase I promoter, an RNA polymerase II promoter, an RNA polymerase III promoter, a T7 promoter, an SP6 promoter or a T3 promoter.

A host cell can be any host cell suitable for cloning, expression, propagation, or production of the virus particle. A host cell can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines (e.g., HEK cells, BHK cells, HeLa cells or CHO cells) or primary mammalian cells. Preferred host cells include HEK cells, in particular HEK293 cells, such as HEK293T (CVCL_0063) or FreeStyle 293-F (CVCL_D603). FreeStyle 293-F cells are commercially available, e.g., from Thermo Fisher Scientific Inc. (Catalogue number 12338026). Preferred host cells also include BHK cells, such as BHK-21 cells (CVCL_1914). Preferred host cells also include WHO Vero RCB 10-87 cells (ATCC CCL 81).

The present invention also relates to a method of producing a virus particle of the invention. The method comprises cultivating a host cell of the invention under conditions suitable for virus particle formation.

The method of producing the virus particle can further comprise introducing into a host cell the cDNA described herein. The method of producing the virus particle can also comprise recovery and/or purification of the virus particle. Such recovery and/or purification methods are well-known to those skilled in the art.

The present invention also relates to a pharmaceutical composition comprising a virus particle disclosed herein. The virus particle preferably comprises an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40. The pharmaceutical composition may further comprise a pharmaceutically acceptable excipient or carrier. A carrier may be e.g., be selected from the group consisting of water, aqueous saline solution, aqueous buffer solution, cell culture medium and combinations of at least two of the foregoing carriers.

The present invention also relates to a virus particle disclosed herein for use in therapy. The virus particle preferably comprises an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40. The virus particle disclosed herein may be for use in the treatment of a cancer or tumor.

The present invention also relates to a use of a virus particle disclosed for the manufacture of a medicament. The virus particle preferably comprises an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40. The medicament is preferably for the treatment of a cancer or tumor.

The present invention also relates to a method of treating a disease comprising administering to a subject a virus particle disclosed herein. The subject is preferably in need thereof. The virus particle is preferably administered in an effective amount. The virus particle preferably comprises an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40. The disease is preferably a cancer or tumor.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as mice, sheep, dogs, horses, cats, cows, rats, pigs, apes such as cynomolgus monkeys and etc., to name only a few illustrative examples. Preferably, the mammal herein is human. The cancer or tumor may be a human or murine cancer or tumor, with a human cancer or tumor being preferred.

An “effective amount” is an amount sufficient to effect beneficial or desired results. An effective amount can be administered in one or more administrations.

The cancer or tumor may be any cancer or tumor disclosed herein. Generally, the cancer or tumor can be selected from the group consisting of carcinoma, melanoma, blastoma, lymphoma and sarcoma.

The term “carcinoma” in the context of the present disclosure should be understood to mean a malignant neoplasia of epithelial origin. A carcinoma is preferably selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, oropharyngeal carcinoma, prostate carcinoma, thyroid carcinoma and cervical carcinoma.

The term “sarcoma” in the context of the present disclosure should be understood to mean a malignant neoplasia of mesodermal origin. A sarcoma can be selected from the group consisting of angiosarcoma, chondrosarcoma, Ewing sarcoma, fibrosarcoma, Kaposi sarcoma, liposarcoma, leiomyosarcoma, malignant fibrous histiocytoma, neurogenic sarcoma, osteosarcoma and rhabdomyosarcoma.

The term “melanoma” in the context of the present disclosure should be understood to mean a malignant neoplasia of melanocytic origin.

The term “lymphoma” in the context of the present disclosure should be understood to mean a malignant neoplasia of lymphocytic origin.

The term “blastoma” in the context of the present disclosure should be understood to mean a malignant neoplasia of embryonic origin.

The cancer or tumor to be treated may be a cold tumor. Preferred cold tumors include breast cancer, ovarian cancer, prostate cancer, pancreatic cancer, and glioblastoma.

The cancer or tumor to be treated may be a hot tumor. Preferred hot tumors include melanoma, bladder cancer, kidney cancer, head and neck cancer, and non-small cell lung cancer.

Preferred cancers or tumors to be treated include melanoma, colon carcinoma, fibrosarcoma, pancreas cancer, thyroid carcinoma, lung cancer, adenocarcinoma, and gastrointestinal cancer.

Generally, the virus particles disclosed herein can be administered via any suitable route that is known to the skilled person. Administration generally includes enteral and parenteral administration. Parenteral administration can include local administration, such as intramuscular, intraperitoneal, subcutaneous, or intratumoral administration. Alternatively, parenteral administration can include systemic administration, in particular intravenous administration, such as via injection or infusion.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms. E.g., the term “comprising” is meant to provide explicit support also for “consisting essentially of” and “consisting of”, the term “consisting essentially of” is meant to provide explicit support also for “comprising” and “consisting of”, the term “consisting of” is meant to provide explicit support also for “consisting essentially of” and “comprising”.

The present invention is further characterized by the following items.

Item 1. A virus particle comprising a lymphocytic choriomeningitis virus (LCMV) S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40, wherein the glycoprotein comprises at least one mutated amino acid residue in comparison with the glycoprotein sequence set forth in SEQ ID NO: 4.

Item 2. The virus particle of item 1, wherein the glycoprotein comprises at least one mutated amino acid residue at position 181 and/or 185 in comparison with the glycoprotein sequence set forth in SEQ ID NO: 4, wherein the glycoprotein preferably comprises at least one mutation selected from Arg 185→Trp and Ile 181→Met in comparison with the glycoprotein sequence set forth in SEQ ID NO: 4.

Item 3. The virus particle of item 1 or 2, wherein the L protein comprises at position 1079 corresponding to the linear polypeptide sequence of SEQ ID NO: 40 an amino acid residue other than Lys, preferably a non-basic amino acid residue, preferably a neutral hydrophilic amino acid residue, preferably an Asn or Gln, preferably a Gln.

Item 4. A virus particle comprising a lymphocytic choriomeningitis virus (LCMV) S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40, wherein the L protein comprises at position 1079 corresponding to the linear polypeptide sequence of SEQ ID NO: 40 an amino acid residue other than Lys.

Item 5. The virus particle of item 4, wherein the L protein comprises at position 1079 corresponding to the linear polypeptide sequence of SEQ ID NO: 40 a non-basic amino acid residue, preferably a neutral hydrophilic amino acid residue, preferably an Asn or Gln, preferably a Gln.

Item 6. The virus particle of item 4 or 5, wherein the glycoprotein comprises at least one mutated amino acid residue in comparison with the glycoprotein sequence set forth in SEQ ID NO: 4, wherein the glycoprotein preferably comprises at least one mutated amino acid residue at position 181 and/or 185 in comparison with the glycoprotein sequence set forth in SEQ ID NO: 4, wherein the glycoprotein preferably comprises at least one mutation selected from Arg 185→Trp and Ile 181→Met in comparison with the glycoprotein sequence set forth in SEQ ID NO: 4.

Item 7. The virus particle of any one of the preceding items, wherein the S segment is derived from LCMV strain WE or a variant thereof and wherein the L segment is derived from LCMV strain Clone13 or a variant thereof.

Item 8. The virus particle of any one of the preceding items, wherein the S segment comprises an open reading frame encoding a nucleoprotein having at least 97% sequence identity to SEQ ID NO: 6.

Item 9. The virus particle of any one of the preceding items, wherein the L segment comprises an open reading frame encoding a Z protein having at least 90% sequence identity to SEQ ID NO: 38.

Item 10. A virus particle comprising a lymphocytic choriomeningitis virus (LCMV) S segment and an LCMV L segment, wherein the S segment comprises an open reading frame encoding a glycoprotein having at least 97% sequence identity to SEQ ID NO: 4 and an open reading frame encoding a nucleoprotein having at least 97% sequence identity to SEQ ID NO: 6, and wherein the L segment comprises an open reading frame encoding an L protein having at least 90% sequence identity to SEQ ID NO: 40 and an open reading frame encoding a Z protein having at least 90% sequence identity to SEQ ID NO: 38.

Item 11. The virus particle of item 10, wherein the glycoprotein comprises at least one mutated amino acid residue in comparison with the glycoprotein sequence set forth in SEQ ID NO: 4, wherein the glycoprotein preferably comprises at least one mutated amino acid residue at position 181 and/or 185 in comparison with the glycoprotein sequence set forth in SEQ ID NO: 4, wherein the glycoprotein preferably comprises at least one mutation selected from Arg 185→Trp and Ile 181→Met in comparison with the glycoprotein sequence set forth in SEQ ID NO: 4.

Item 12. The virus particle of item 10 or 11, wherein the L protein comprises at position 1079 corresponding the linear polypeptide sequence of SEQ ID NO: 40 an amino acid residue other than Lys, preferably a non-basic amino acid residue, preferably a neutral hydrophilic amino acid residue, preferably an Asn or Gln, preferably a Gln.

Item 13. The virus particle of any one of the preceding items, wherein the virus particle is capable of promoting a stronger reduction of growth of a tumor as compared to LCMV strain WE and/or a virus particle comprising the same S segment as the virus particle and an L segment as shown in SEQ ID NO: 2.

Item 14. The virus particle of any one of the preceding items, wherein the virus particle is capable of promoting a stronger reduction of growth of a cold tumor as compared to LCMV strain WE and/or a virus particle comprising the same S segment as the virus particle and an L segment as shown in SEQ ID NO: 2.

Item 15. The virus particle of any one of the preceding items, wherein the virus particle is capable of inducing a stronger adaptive immune activation as compared to LCMV strain WE and/or a virus particle comprising the same S segment as the virus particle and an L segment as shown in SEQ ID NO: 2.

Item 16. The virus particle of any one of the preceding items, wherein the virus particle is capable of promoting a reduction of growth of a warm tumor that is at least as strong as compared to LCMV strain WE and/or a virus particle comprising the same S segment as the virus particle and an L segment as shown in SEQ ID NO: 2.

Item 17. The virus particle of any one of the preceding items, wherein the virus particle is capable of inducing decreased levels of an interferon upon systemic administration compared to LCMV strain WE, Armstrong, Clone13 and/or a virus particle comprising the same S segment as the virus particle and an L segment as shown in SEQ ID NO: 2.

Item 18. The virus particle of any one of the preceding items, wherein the virus particle is capable of inducing a lower concentration of one or more Th1- and pro-inflammatory cytokines compared to LCMV strain WE, Armstrong, Clone13 and/or a virus particle comprising the same S segment as the virus particle and an L segment as shown in SEQ ID NO: 2.

Item 19. The virus particle of any one of the preceding items, wherein the virus particle is capable of inducing a lower concentration of one or more Th1- and pro-inflammatory cytokines compared to LCMV strain WE, Armstrong, Clone13 and/or a virus particle comprising the same S segment as the virus particle and an L segment as shown in SEQ ID NO: 2.

Item 20. The virus particle of any one of the preceding items, wherein the virus particle is less pathogenic in a mouse compared to LCMV strain WE.

Item 21. The virus particle of any one of the preceding items, wherein the virus particle is capable of inducing a reduced degree of liver pathology in a mouse compared to LCMV strain WE and/or a virus particle comprising the same S segment as the virus particle and an L segment as shown in SEQ ID NO: 2.

Item 22. The virus particle of any one of the preceding items, wherein the virus particle shows reduced replication in neuron cells compared to LCMV strain WE and/or a virus particle comprising the same S segment as the virus particle and an L segment as shown in SEQ ID NO: 2.

Item 23. The virus particle of any one of the preceding items, wherein the virus particle shows increased replication in tumor cells compared to LCMV strain WE.

Item 24. The virus particle of any one of the preceding items, wherein the virus particle shows reduced replication in a healthy organ compared to LCMV strain WE and/or a virus particle comprising the same S segment as the virus particle and an L segment as shown in SEQ ID NO: 2.

Item 25. The virus particle of any one of the preceding items, wherein the S segment comprises an open reading frame encoding a glycoprotein that has at least about 98%, preferably at least about 98.5%, preferably at least about 99%, preferably at least about 99.1%, preferably at least about 99.2%, preferably at least about 99.3%, preferably at least about 99.4%, preferably at least about 99.5%, preferably at least about 99.5%, preferably at least about 99.7% sequence identity, or is preferably identical, to a sequence set forth in SEQ ID NO: 4 or 14.

Item 26. The virus particle of any one of the preceding items, wherein the S segment comprises an open reading frame comprising a sequence that has at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, or is preferably identical, to a sequence set forth in SEQ ID NO: 3 or 13.

Item 27. The virus particle of any one of the preceding items, wherein the L segment comprises an open reading frame encoding an L protein having at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, or is preferably identical, to a sequence set forth in SEQ ID NO: 30 or 40.

Item 28. The virus particle of any one of the preceding items, wherein the L segment comprises an open reading frame comprising a sequence that has at least about 83%, preferably at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, preferably at least about 99.8%, preferably at least about 99.9% sequence identity, or is preferably identical, to a sequence set forth in SEQ ID NO: 29 or 39.

Item 29. The virus particle of any one of the preceding items, wherein the S segment comprises an open reading frame encoding a nucleoprotein, wherein said nucleoprotein has at least about 98%, preferably 98.5%, preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is preferably identical, to a sequence set forth in any one of in SEQ ID NO: 6 or 16.

Item 30. The virus particle of any one of the preceding items, wherein the S segment comprises an open reading frame comprising a sequence that has at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is preferably identical, to a sequence set forth in SEQ ID NO: 5 or 15.

Item 31. The virus particle of any one of the preceding items, wherein the L segment comprises an open reading frame encoding a Z protein having at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is preferably identical, to a sequence set forth in SEQ ID NO: 28 or 38.

Item 32. The virus particle of any one of the preceding items, wherein the L segment comprises an open reading frame comprising a sequence that has at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or that is identical, to a sequence set forth in SEQ ID NO: 27 or 37.

Item 33. The virus particle of any one of the preceding items, wherein the S segment comprises a sequence that has at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, preferably at least about 99.7%, preferably at least about 99.8%, preferably at least about 99.9% sequence identity, or is preferably identical, to a sequence set forth in SEQ ID NO: 1 or 11.

Item 34. The virus particle of any one of the preceding items, wherein the L segment comprises a sequence that has at least about 83%, preferably at least about 84%, preferably at least about 85%, preferably at least about 86%, preferably at least about 87%, preferably at least about 88%, preferably at least about 89%, preferably at least about 90%, preferably at least about 91%, preferably at least about 92%, preferably at least about 93%, preferably at least about 94%, preferably at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, preferably at least about 99.7%, preferably at least about 99.8%, preferably at least about 99.9% sequence identity, or is preferably identical, to a sequence set forth in SEQ ID NO: 21 or 31.

Item 35. The virus particle of any one of the preceding items, wherein the virus particle is infectious.

Item 36. The virus particle of any one of the preceding items, wherein the virus particle is replication competent.

Item 37. The virus particle of any one of the preceding items, wherein the virus particle is attenuated.

Item 38. The virus particle of any one of the preceding items, wherein the ORF encoding the glycoprotein is under control of a 5′ untranslated region (UTR).

Item 39. The virus particle of any one of the preceding items, wherein the ORF encoding the L protein is under control of a 3′ untranslated region (UTR).

Item 40. The virus particle of any one of the preceding items, wherein the ORF encoding the nucleoprotein is under control of a 3′ untranslated region (UTR).

Item 41. The virus particle of any one of the preceding items, wherein the ORF encoding the Z protein is under control of a 5′ untranslated region (UTR).

Item 42. The virus particle of any one of the preceding items, wherein the virus particle has a bi-segmented genome.

Item 43. The virus particle of any one of the preceding items, wherein the virus particle is an arenavirus particle.

Item 44. The virus particle of any one of the preceding items, wherein the virus particle is an LCMV particle.

Item 45. The virus particle of any one of the preceding items, wherein the virus particle does not comprise an ORF from an organism other than an LCMV.

Item 46. A host cell comprising an LCMV S segment and an LCMV L segment as defined in any one of the preceding items.

Item 47. A host cell comprising cDNA of (a) an ORF encoding a glycoprotein as defined in any one of items 1-45, an ORF encoding an L protein as defined in any one of items 1-45, an ORF encoding a nucleoprotein as defined in any one of items 1-45, and an ORF encoding a Z protein as defined in any one of items 1-45; and/or (b) an LCMV S segment and an LCMV L segment as defined in any one of items 1-45.

Item 48. The host cell of item 46 or 47, wherein the host cell is a HEK293 cell.

Item 49. A method of producing a virus particle of any one of items 1-45 comprising cultivating the host cell of any one of items 46-48 under conditions suitable for virus particle formation.

Item 50. The method of item 49 further comprising recovery and/or purification of the virus particle.

Item 51. A pharmaceutical composition comprising a virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40.

Item 52. The pharmaceutical composition of item 51, wherein the virus particle is a virus particle of any one of items 1-45.

Item 53. A virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40 for use in therapy.

Item 54. The virus particle for the use of item 53, wherein the virus particle is a virus particle of any one of items 1-45.

Item 55. The virus particle for the use of item 53 or 54, wherein the use is in the treatment of cancer.

Item 56. The virus particle for the use of any one of items 53-55, wherein the use is in the treatment of a human subject.

Item 57. The virus particle for the use of any one of items 53-56, wherein the use is in the treatment of a human cancer.

Item 58. The virus particle for the use of any one of items 53-57, wherein the use is in the treatment of a cold tumor.

Item 59. The virus particle for the use of any one of items 53-58, wherein the use is in the treatment of a hot tumor.

Item 60. The virus particle for the use of any one of items 53-59, wherein the use is in the treatment of a tumor selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, colorectal carcinoma, hepatocellular carcinoma, testicular carcinoma, colon carcinoma, tumor of the bladder, bladder carcinoma, tumor of the bladder, hepatocellular carcinoma, lung carcinoma, lung carcinoma, endometrial carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, oropharyngeal carcinoma, prostate carcinoma, thyroid carcinoma, Cervical cancer, angiosarcoma, chondrosarcoma, Ewing sarcoma, fibrosarcoma, Kaposi sarcoma, liposarcoma, leiomyosarcoma, malignant fibrosis histiocytoma, lymphoma, leukemia, neurogenic sarcoma, osteosarcoma and rhabdomyosarcoma.

Item 61. A use of a virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40 for the manufacture of a medicament, wherein the medicament is preferably for the treatment of cancer.

Item 62. A method of treating a disease comprising administering to a subject in need thereof an effective amount of a virus particle comprising an LCMV S segment and an LCMV L segment, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40, wherein the disease is preferably cancer.

### EXAMPLES

### Mice

Analysis of anti-tumoral effects was performed by using wild type animals on C57BL/6 background. For in vivo analysis of LCMV induced effects wildtype mouse strains C57BL/6, BALB/c or FVB/N were used. C57BL/6 and BALB/c strains are resistant to lethal infection caused by LCMV. FVB/N mice represent a highly susceptible mouse strain which develops a hemorrhagic fever like illness after infection with LCMV strain Clone13. Habitus was observed and weight was measured subsequent to LCMV treatment.

### Cell Lines

MC57G (CVCL_4985) is a murine fibrosarcoma cell line, which show robust LCMV replication. Tramp-C2 is a murine prostate gland cell line (CVCL_3615). B16 (CVCL_F936) and B16-F10 (B16F10, CVCL_0159) are murine melanoma cell lines. B16-Ova represents the respective B16 cell line expressing ovalbumin as antigen. MC-38 (MC38, CVCL_B288) is a murine colon adenocarcinoma cell line. JAWSII is a murine, spontaneously immortalized cell line, which can be differentiated into dendritic like cells by treatment with GM-CSF (CVCL_3727). 511950 is a genetically engineered murine primary pancreas cancer cell line passaged less than 20 times (Mazur et al. (2015) Nature medicine 21 (10): 1163-1171). Bone marrow derived dendritic cells (BMDCs) were differentiated from murine bone marrow in the presence of GM-CSF.

C643 (CVCL_5969) is a human anaplastic thyroid carcinoma cell line. NCI-H1975 (H1975, CVCL_1511) is a human lung adenocarcinoma cell line. A549 is human adenocarcinoma cell line (CVCL_0023). FTC133 is a human thyroid carcinoma cell line (CVCL_1219). GIST-T1 is a human gastrointestinal tumor cell line derived from pleural effusion (CVCL_4976). Hek293T (CVCL_0063) is a human embryonic kidney cell line, which shows robust LCMV replication. FreeStyle 293-F (CVCL_D603) is a derivative of Hek293 cells, propagated in Freestyle Medium. FreeStyle 293-F cells are the desired producer cell line for LCMV production.

UKE-Ma-Mel-51 (Mamel51, CVCL_A186) and UKE-Ma-Mel-86a (Ma-Mel-86a, CVCL_A221) are human primary melanoma cell lines derived from lymph node metastasis provided by Prof. Dr. Paschen, Klinik für Dermatologie, UK Essen. Primary human neurons were differentiated from human neuronal progenitor cells (NPC) kindly provided by Prof. Dr. Gopalakrishnan, HHU Düsseldorf. SkMM (Human Skeletal muscle myoblasts, Lonza CC-2580) are human primary skeletal myoblasts. Myotubes were differentiated from SkMM in the presence of SkGM-2, Horse Serum, Glutamine, Gentamicin and Dexamethasone. Differentiation was verified by staining with Anti-Myosin 4 AK (eBioscience, Clone MF-20).

BHK-21 cells represent a spontaneous immortalized fibroblast hamster cell line (CVCL_1914).

### Viruses

The LCMV strain WE was obtained from the laboratory of Prof. Zinkernagel (Experimental Immunology, Zurich, Switzerland) and has been propagated in L929 cells or BHK-21 cells since 2008.

The LCMV strains Clone13 and Armstrong 53b were obtained from S. Basta, Queens University and R. M. Zinkernagel, University of Zurich, respectively. The LCMV reassortants WE-Cl13(L), P52-Cl13(L), P52-WE(L) and Cl13-WE(L) were rescued by transient transfection entirely from plasmids as described (Flatz et al. (2006) Proc Natl Acad Sci USA 103 (12): 4663-4668). The viruses consist of the S-Segment of the LCMV strain WE or WE-derived P52 and the L-Segment of the LCMV strains Clone13 or WE. Viruses were propagated on BHK-21 or Freestyle 293F cells.

### Generation of Recombinant Viruses

LCMV recombinant and reassortant viruses were generated entirely from plasmids. BHK-21 cells were transiently transfected with plasmids coding for the LCMV S segment, L segment as well as helper plasmids coding for the L polymerase and the nucleoprotein. The LCMV reassortant WE-Cl13(L) was generated using a WE S segment and the Clone13 L segment plasmid. The LCMV P52-Cl13(L) was rescued using a LCMV P52 S segment plasmid, which harbors two coding mutations in the glycoproteins, 1181M and R185W, and a Clone13 L segment plasmid. The LCMV P52-WE(L) was generated using a LCMV P52 S segment plasmid and a LCMV WE L segment plasmid. The Cl13-WE(L) was generated using a LCMV Clone13 S segment plasmid and a WE L segment plasmid.

### Determination of LCMV Infected Cells and Infiltration of Immune Cell in Tumor Tissue Ex Vivo by Immunofluorescence

Immunohistofluorescence was used to detect LCMV and immune cell distribution in tumor tissues of LCMV treated or control treated tumor bearing mice. Tumor biopsies were cut into 7 μm thick sections and stained with a fluorochrome-labelled anti-LCMV-NP antibody (clone VL4), and antibody against CD8 (eBioscience) and visualized with a fluorescence microscope (Keyence) and photographed with an integrated CCD camera.

### Determination of LCMV in Supernatant or Organs by Plaque-Forming Assay

To analyze LCMV replication capacity, tumor and primary cells were seeded out in 24-well plates (100.000 cells/well) and infected with LCMV at MOIs shown. Supernatant was harvested 24 h, 48 h or 72 h after infection. Analysis of LCMV production by FreeStyle 293-F cells was performed by seeding out 1.5×10⁶ cells per mL and infection with LCMV at MOI=0.001. Supernatant was harvested after 12, 24, 30, 34, 40, 48, 72 and 96 hours post infection. Analysis of LCMV biodistribution was performed in infected tumor bearing and non-tumor bearing animals. Organs of infected mice were dissected and homogenized in cell culture medium. The supernatants were titrated and incubated on MC57G cells (100.000 cells/well). Methylcellulose overlay was added after three to four hours. LCMV infected plaques were stained with anti LCMV-NP antibody (clone VL4). Plaques were counted and infectious particles were determined as virus titer per mL of supernatant or per organ.

### Determination of LCMV Infected Cells by Flow Cytometry

To analyze LCMV infectivity in tumor and primary cells, cells were seeded out in 96-well plates, infected with LCMV at MOI=0.1 or left untreated and incubated one hour on ice. After one hour, cells were transferred to 37 °C. 20 h post infection, cells were harvested and intracellular LCMV staining was performed using fluorescent labelled LCMV NP antibody (clone VL4). Cells were analyzed via flow cytometry (LSR Fortessa, BD) and results were displayed as percent LCMV infected cells or mean fluorescent intensity (MFI).

### Determination of Cytokine Production by Flow Cytometry

The intensity of immune activation by LCMV was analyzed by measuring cytokines in serum. Therefore, serum of naïve or infected mice, with or without tumors, was analyzed via LegendPlex analysis according to manufacturer's protocol (BioLegend).

### Clinical Chemistry

The induction of liver pathology by LCMV was determined by measuring alanine aminotransferase (ALT) and aspartate aminotransferase (AST). Mice were bled on various days after LCMV infection and serum was extracted. Analysis of ALT and AST values was performed at Zentrallabor, Universitätsklinkum Essen or Zentralinstitut für Klinische Chemie und Laboratoriumsdiagnostik, HHU Düsseldorf.

### Analysis of Immune Cells by Flow Cytometry

To determine the effect of LCMV therapy immune cells were analyzed via flow cytometry (LSR Fortessa, BD). Thrombocytes were detected by their specific size and granularity. T cells were stained with specific fluorescent labelled antibodies for CD8, CD4, CD3 (eBioscience). LCMV specific T cells were stained using tetramer staining (NIH, Tetramer Facility).

### Tumor Growth and Treatments

To measure the anti-tumoral effect of LCMV therapy, C57BL/6 mice (6-12 weeks old) were subcutaneously injected with 1×10⁶ tumor cells (in 100 μL) into the right or left flank. After a visible tumor formation, animals were LCMV or control treated, and the mean tumor volume was determined. Endpoint analysis of tumor mass was performed by measuring the total weight (in g) of the tumor.

### Statistical Analysis

For column diagrams the mean values were compared using unpaired two-sample Student's t-test. Tumor growth curves were compared using two-way ANOVA with Skidak's multiple comparison test. Data are presented as mean±SEM. The level of statistical significance was determined to be p<0.05.

### Investigations

### Example 1

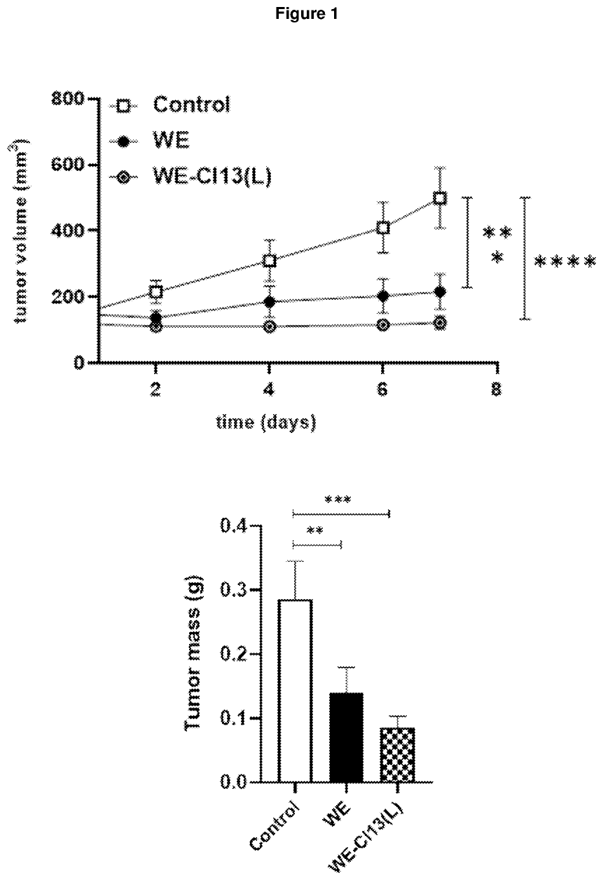

C57BL/6 mice were subcutaneously treated with B16 melanoma cells and intravenously infected with 2×10⁴ PFU of the LCMV strains WE or WE-Cl13(L) or were control treated (n=5 animals per group). Tumor growth was measured over seven days. Tumor mass was taken at finalization (d7) (FIG. 1).

Thereby it was proven that LCMV WE-CL13(L) shows an increased antitumoral effect compared to wildtype LCMV strain WE in the non-immunogenic cold B16 melanoma tumor model. This model is an example for a cold tumor.

### Example 2

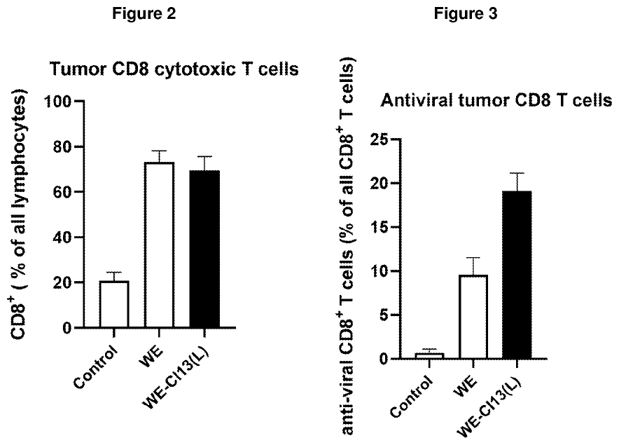

C57BL/6 mice were subcutaneously treated with B16 melanoma cells and intravenously infected with 2×10⁴ PFU of LCMV strains WE or WE-Cl13(L) or were control treated (n=5 animals per group). Mice were sacrificed at day seven after infection. Tumor resident cytotoxic CD8⁺ T cells were stained with fluorescently labelled antibody (CD8 eBioscience) and analyzed via flow cytometry (FIG. 2).

Thereby it was proven that LCMV strain WE-Cl13(L) enhances T cell infiltration into the tumor tissue.

### Example 3

C57BL/6 mice were subcutaneously treated with B16 melanoma cells and intravenously infected with 2×10⁴ PFU of LCMV strains WE or WE-Cl13(L) or were control treated (n=5 animals per group). Mice were sacrificed at day seven after infection. Tumor resident cytotoxic, antiviral CD8⁺ GP33-tetramer positive T cells were analyzed via flow cytometry on day seven after infection (FIG. 3).

Thereby it was proven that LCMV strain WE-Cl13(L) induces a stronger adaptive immune activation than wildtype LCMV strain WE.

### Example 4

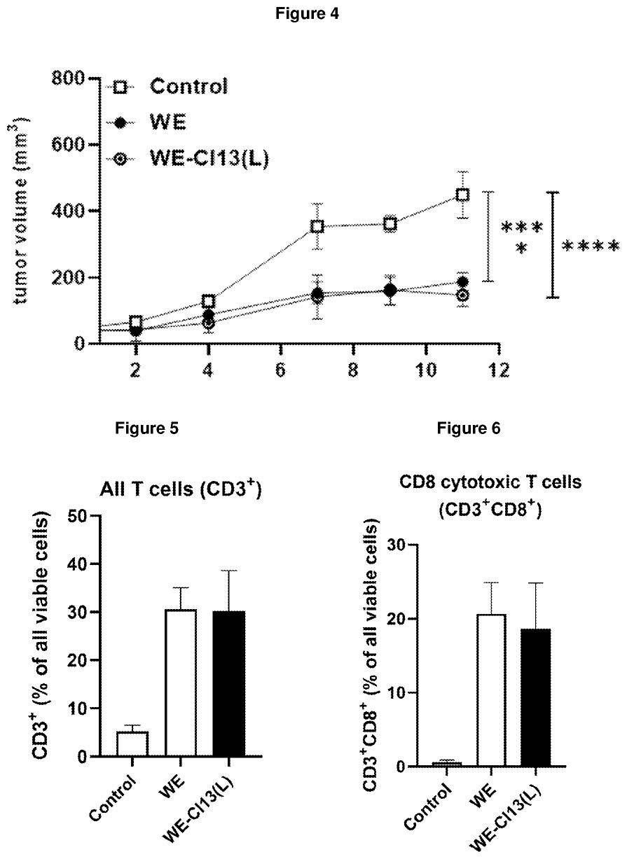

C57BL/6 mice were subcutaneously treated with MC38 colon carcinoma cells. Upon tumor formation mice were intravenously infected with 2×10⁴ PFU of LCMV strains WE or WE-Cl13(L) or were control treated (n=4 animals per group). Tumor growth was observed for eleven days (FIG. 4).

Thereby it was proven that LCMV strain WE-Cl13(L) shows a strong tumor growth inhibiting effect in the immunogenic warm MC38 tumor model. This is a model for a warm tumor.

### Example 5

C57BL/6 mice were subcutaneously treated with MC38 colon carcinoma cells. Upon tumor formation mice were intravenously infected with 2×10⁴ PFU of LCMV strains WE or WE-Cl13(L) or were control treated (n=4 animals per group). Tumor tissue was dissected on day eleven after infection. Tumor infiltrating T cells (CD3⁺) were stained via fluorescent labelled antibody (CD3, eBioscience) and measured via flow cytometry (Example 5).

Thereby it was proven that LCMV strain WE-Cl13(L) induces a strong T cell infiltration into the tumor.

### Example 6

C57BL/6 mice were subcutaneously treated with MC38 cells and intravenously infected with 2×10⁴ PFU of LCMV strains WE or WE-Cl13(L) or were control treated (n=4 animals per group). Tumor tissue was dissected on day eleven after infection. Tumor infiltrating cytotoxic T cells (CD3⁺CD8⁺) were stained via fluorescent labelled antibodies (CD3, CD8, eBioscience) and analyzed via flow cytometry (FIG. 6).

Thereby it was proven that LCMV strain WE-Cl13(L) strongly enhances cytotoxic T cell infiltration into the tumor.

### Example 7

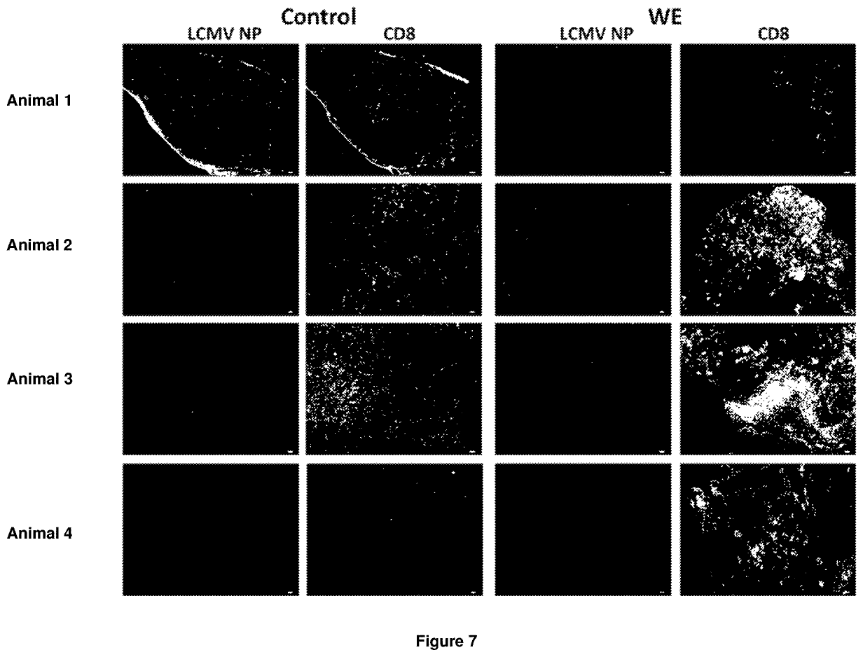

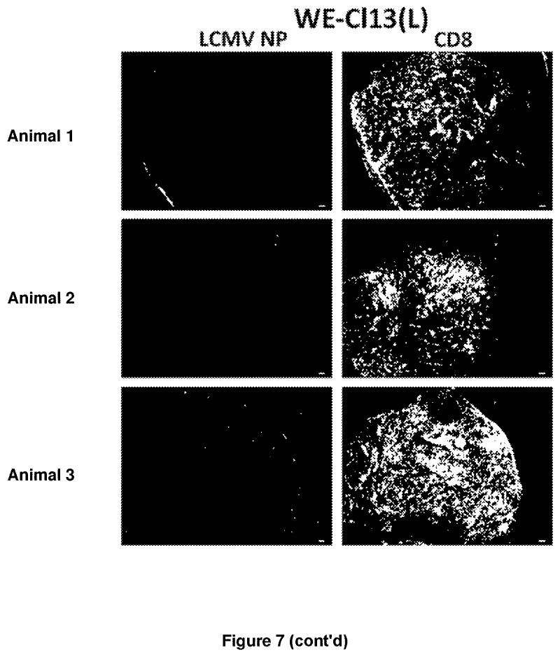

C57BL/6 mice were subcutaneously treated with MC38 cells and intravenously infected with 2×10⁴ PFU of LCMV strains WE (n=4) or WE-Cl13(L) (n=3) or were control treated (n=4). Tumor tissue was histologically analyzed. Tumor sections (7 μm) were stained with fluorescent labelled antibodies for LCMV nucleoprotein (LCMV NP, clone VL4) and CD8⁺ T cells (CD8) (FIG. 7).

Thereby it was proven that LCMV strain WE-Cl13(L) enhances cytotoxic T cell infiltration into the tumor independent of a specific local virus presence.

### Example 8

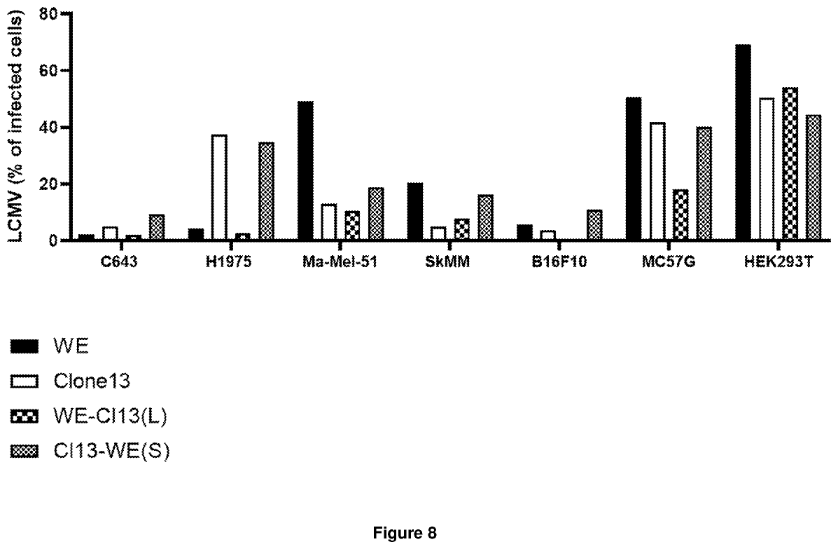

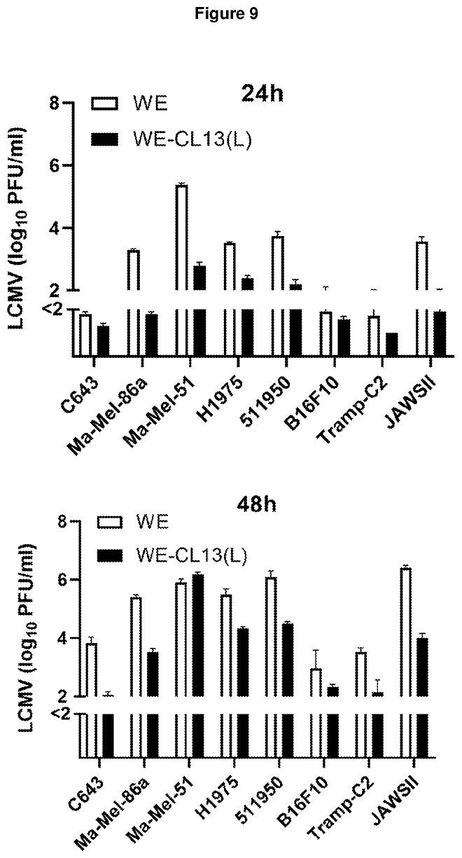

C643, H1975, Ma-Mel-51, SkMM, B16F10, MC57G and HEK293T cells were seeded out (100.000 cells/well) and infected with LCMV strains WE, Clone13, WE-Cl13(L) (WE S-Segment, Clone13 L-Segment) and Clone13-WE(L) (Clone13 S-Segment, WE L Segment) at MOI=0.1. Cells were cultured for 20 hours and intracellularly stained for presence of LCMV nucleoprotein via fluorescent labelled antibody (NP, Clone VL4). The percentage of infected cell from all cells was analyzed via flow cytometry (FIG. 8).

Thereby it was proven that the Clone13 L-Segment is responsible for the attenuation of the virus on all tumor cell lines but does not strongly decrease infection of producer cell line HEK293.

### Example 9

C643, Ma-Mel-86a, Ma-Mel-51, 511950, B16F10, Tramp-C2 and JAWSII cells were seeded out (100.000 cells/well) and infected with LCMV strains WE and WE-Cl13(L) at MOI=0.1. 24 and 48 hours post infection supernatants were harvested and analyzed for LCMV replication via LCMV focus forming assay (Example 9).

Thereby it was proven that LCMV strain WE-Cl13(L) replication capacity is attenuated on tumor cells and antigen presenting cells (JAWSII).

### Example 10

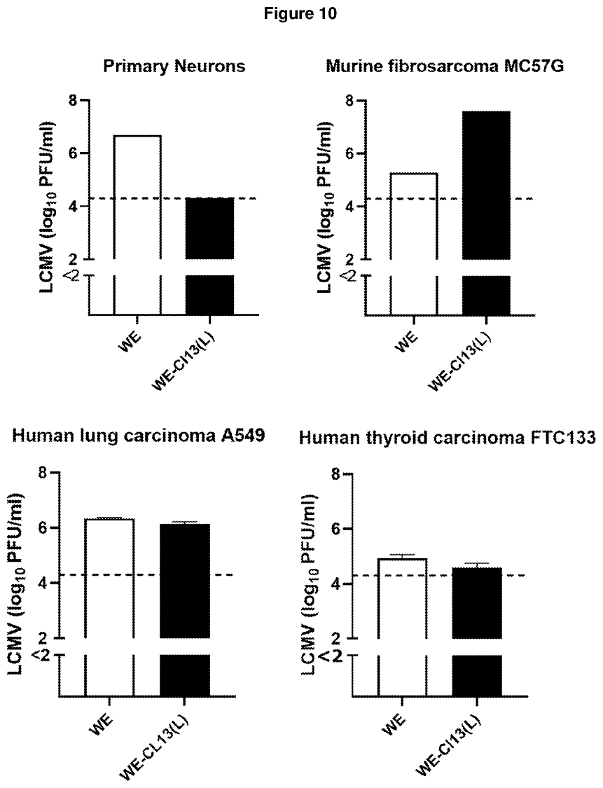

Primary human neurons, murine MC57G cells, human lung adenocarcinoma cell line A549 and human thyroid carcinoma cell line FTC133 were seeded out (100.000 cells/well) and infected at MOI=0.1 with LCMV WE and WE-Cl13(L). Supernatant was harvested after 48 hours and analyzed for LCMV replication via focus forming assay (FIG. 10).

Thereby it was proven that LCMV strain WE-Cl13(L) shows lack of neurotropism but simultaneously enhanced replication in susceptible tumor cells such as the murine fibrosarcoma cell line MC57G and robust replication on human cancer cell lines A549 and FTC133 compared to LCMV strain WE.

### Example 11

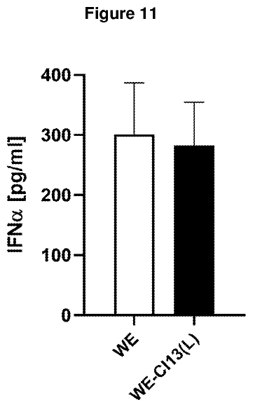

Bone marrow derived dendritic cells were infected with LCMV strains WE and WE-Cl13. Supernatants were harvested and analyzed for Interferon alpha protein via commercial Interferon alpha ELISA-Kit (FIG. 11).

Thereby it was proven that LCMV strain WE-Cl13(L) induces similar interferon response to LCMV WE upon in vitro infection of dendric cells.

### Example 12

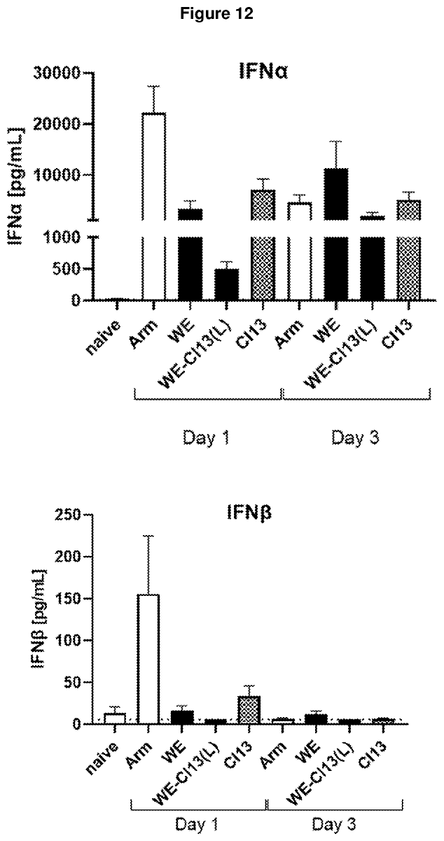

C57BL/6 mice were intravenously infected with 2×10⁴ PFU of LCMV strains Armstrong (Arm, n=3), WE (n=3), WE-Cl13(L) (n=3) or Clone13 (Cl13, n=3) or left untreated (n=12). Blood was taken at day one and day three after infection. Serum cytokine concentrations of IFNα and IFNβ were analyzed via LegendPlex assay (BioLegend) (FIG. 12).

Thereby it was proven that LCMV WE-Cl13(L) reassortant induces decreased levels of Interferons in mice upon systemic administration.

### Example 13

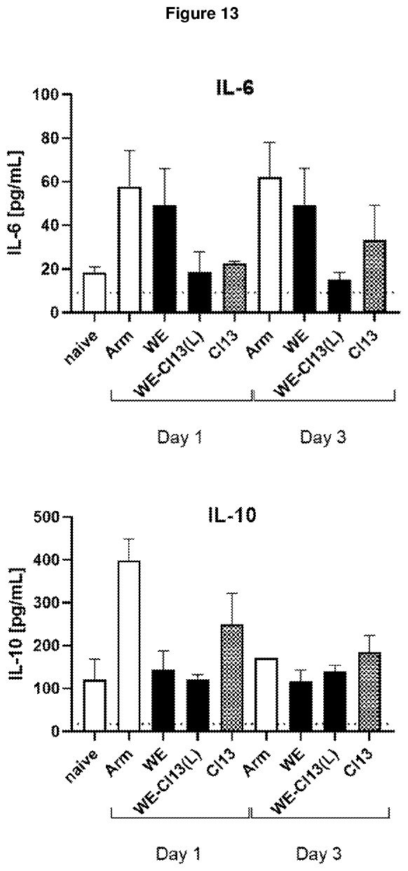

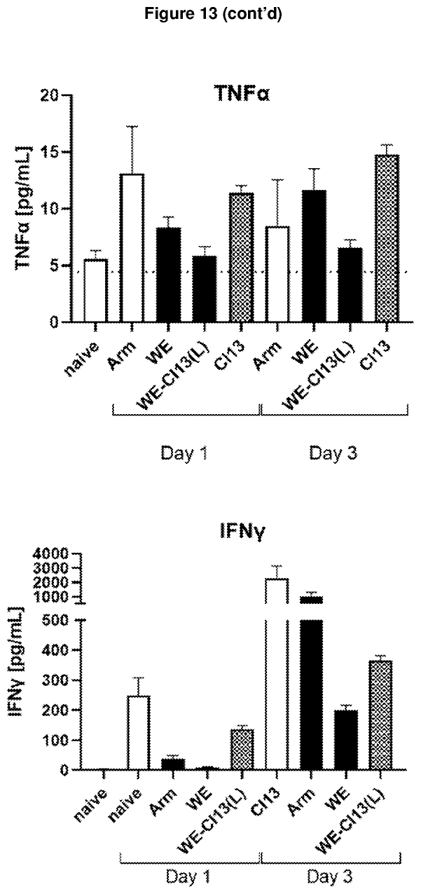

C57BL/6 mice were intravenously infected with 2×10⁴ PFU of LCMV strains Armstrong (Arm, n=3), WE (n=3), WE-Cl13(L) (n=3) or Clone13 (Cl13, n=3) or left untreated (n=12). Blood was taken at day one and day three after infection. Serum cytokine concentrations of IL-6, Il-10, TNFα and IFNγ were analyzed via LegendPlex assay (BioLegend) (FIG. 13).

Thereby it was proven, that LCMV strain WE-CL13(L) induces lower concentrations of Th1- and pro-inflammatory cytokines in mice.

### Example 14

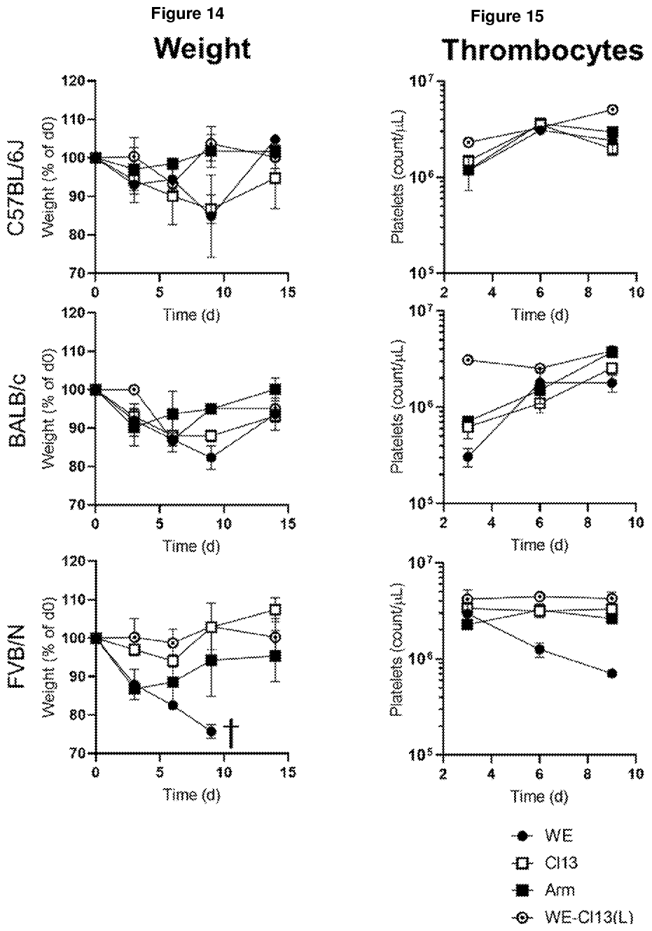

Wild type C57BL/6 or BALB/c mice or the highly susceptible mouse strain FVB/N were intravenously infected with 2×10⁶ PFU of LCMV strains WE, Cl13 (Clone13), Arm (Armstrong) or WE-Cl13(L) (n=3). Weight was measured for a time span of 14 days. The weight was analyzed as percentage of weight on day of infection (FIG. 14).

Thereby it was proven that LCMV reassortant WE-Cl13(L) does not induce lethal pathologies in the highly susceptible mouse strain FVB/N compared to wild type LCMV stain WE.

### Example 15

Wild type C57BL/6 or BALB/c mice or the highly susceptible mouse strain FVB/N were intravenously infected with 2×10⁶ PFU of LCMV strains WE, Cl13 (Clone13), Arm (Armstrong) or WE-Cl13(L) (N=3). Mice were bled at days three, six and nine. Platelets (Thrombocytes) were measured in full blood via flow cytometry. Total count of platelets per microliter of blood was analyzed (FIG. 15).

Thereby it was shown that LCMV strain WE-Cl13(L) does not induce thrombocytopenia in highly susceptible FVB/N and other mouse strains.

### Example 16

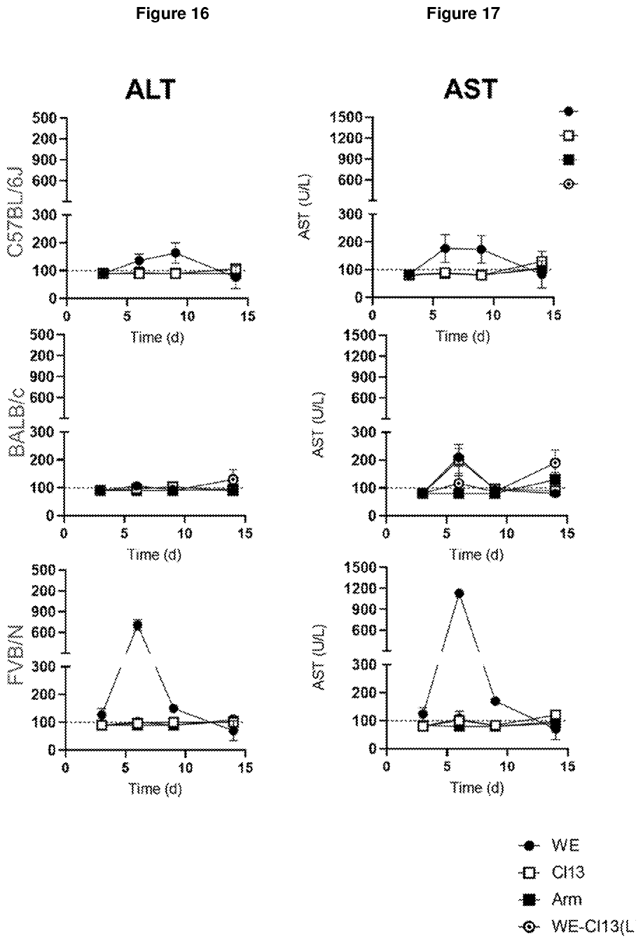

Wild type C57BL/6 or BALB/c mice or the highly susceptible mouse strain FVB/N were intravenously infected with 2×10⁶ PFU of LCMV strains WE, Cl13 (Clone13), Arm (Armstrong) or WE-Cl13(L) (N=3). Mice were bled at days three, six, nine and fourteen. Serum levels of alanine aminotransferase (ALT) were analyzed (FIG. 16).

Thereby it was shown that LCMV WE-Cl13(L) does not induce liver pathology in highly susceptible FVB/N and other mouse strains.

### Example 17

Wild type C57BL/6 or BALB/c mice or the highly susceptible mouse strain FVB/N were intravenously infected with 2×10⁶ PFU of LCMV strains WE, Cl13 (Clone13), Arm (Armstrong) or WE-Cl13(L) (N=3). Mice were bled at days three, six, nine and fourteen. Serum levels of aspartate aminotransferase (AST) were analyzed (FIG. 17).

Thereby it was shown that LCMV WE-Cl13(L) does not induce liver pathology in highly susceptible FVB/N and other mouse strains.

### Example 18

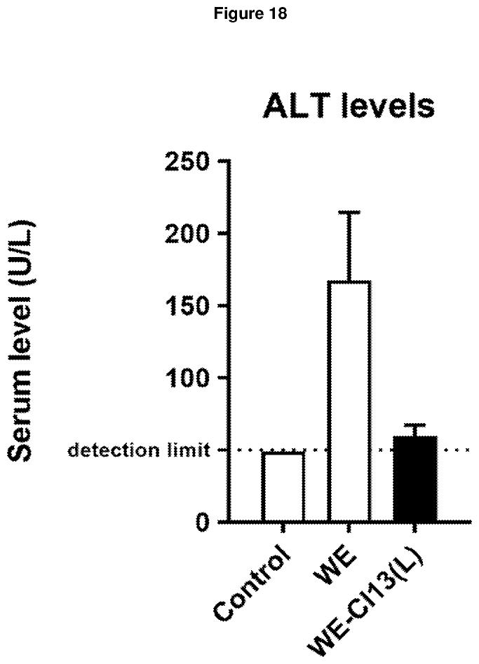

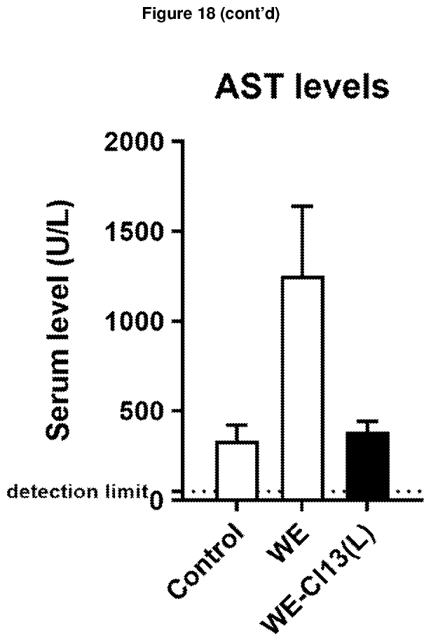

B16 melanoma tumor cells were injected subcutaneously into C57BL/6 mice. Mice were treated with 2×10⁴ PFU of LCMV strains WE (n=4) or WE-Cl13(L) (n=5) or control treated (n=6). Blood was drawn at day seven after infection and serum levels of ALT and AST as indicators of liver pathology were analyzed (FIG. 18).

Thereby it was proven that LCMV strain WE-CL13(L) does not induce liver pathology after systemic administration in the presence of LCMV replication in the tumor.

### Example 19

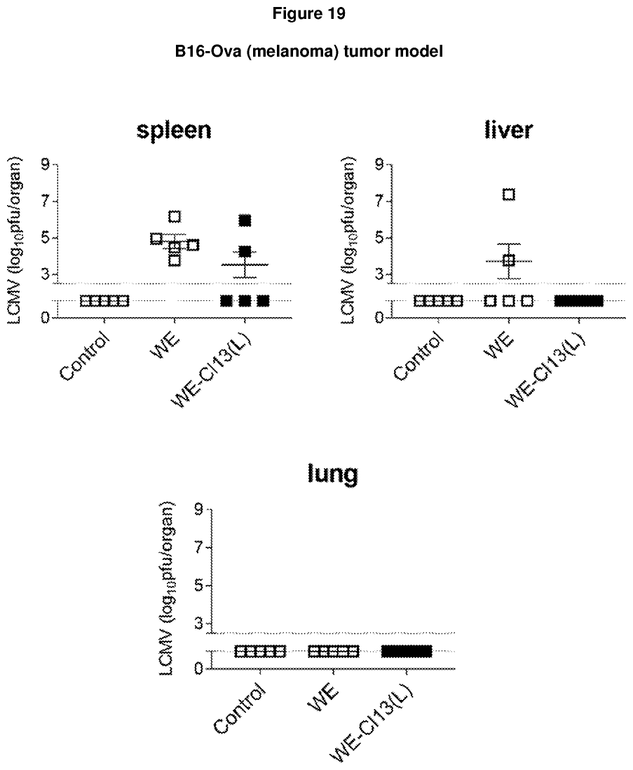

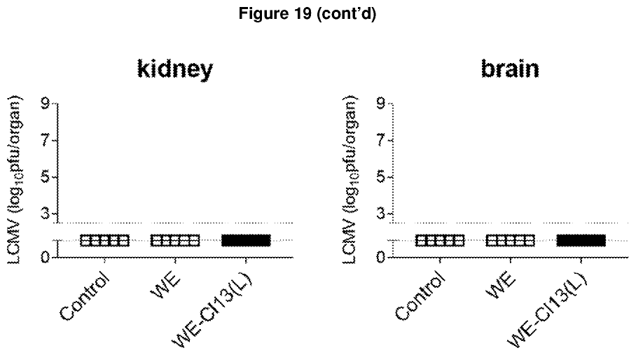

OVA expressing B16 tumor cells were injected in C57BL/6 mice subcutaneously. Mice were treated with 2×10⁴ PFU of LCMV strains WE or WE-Cl13(L) intravenously or control treated (n=5). Mice were sacrificed on day seven after treatment and spleen, liver, lung, kidney and brain were analyzed for replicating LCMV particles via focus forming assay (FIG. 19).

Thereby it was shown that LCMV strain WE-Cl13(L) shows significantly decreased replication in healthy organs.

### Example 20

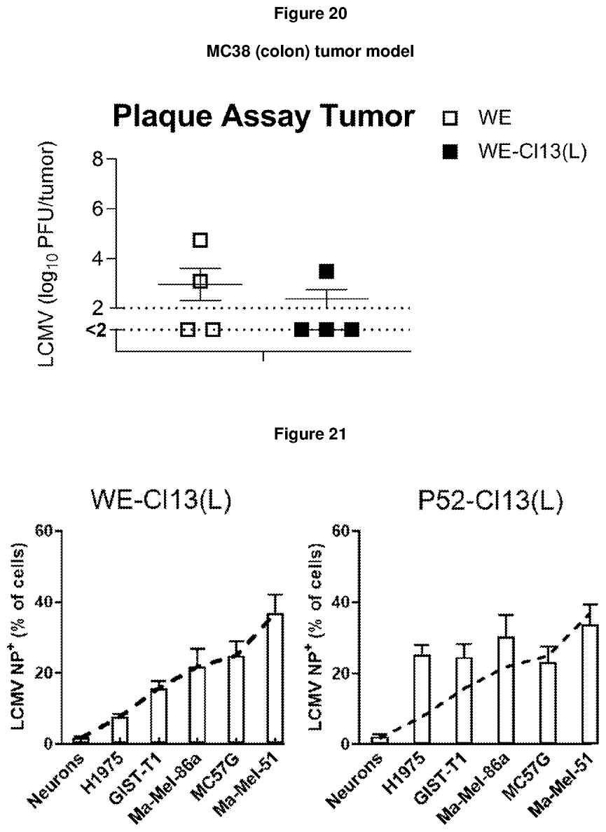

Murine MC38 tumor cells were subcutaneously transplanted in C57BL/6 mice. Mice were treated with 2×10⁴ PFU of LCMV strains WE (n=4) or WE-Cl13(L) (n=4) via intravenous injection or control treated. Tumor tissue was analyzed for LCMV replication via focus forming assay on day eleven after infection (FIG. 20).

Thereby it was proven that LCMV strain WE-Cl13(L) replicates equally to LCMV strain WE in tumor tissue.

### Example 21

Primary human neurons, lung adenocarcinoma cell line H1975, gastrointestinal cancer cell line GIST-T1, primary melanoma cell line Ma-Mel-86a, murine fibrosarcoma MC57G and primary melanoma cell line Ma-Mel-51 were seeded out (100.00 cells/well) and infected with MOI=0.1 of LCMV strains WE, WE-Cl13(L) or P52-Cl13(L). After 20 h, cells were intracellularly stained for LCMV nucleoprotein (NP, clone VL4). The percentage of LCMV infected cells (LCMV NP⁺) was analyzed via flow cytometry (FIG. 21).

Thereby it was proven that LCMV reassortants LCMV WE-Cl13(L) and LCMV P52-Cl13(L) do not replicate in human neurons but that LCMV strain P52-Cl13(L) shows increased infectivity and replication in human and murine tumor cells.

### Example 22

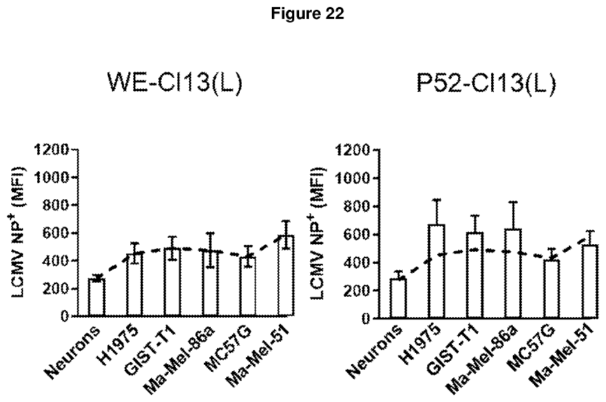

Primary human neurons, lung adenocarcinoma cell line H1975, gastrointestinal cancer cell line GIST-T1, primary melanoma cell line Ma-Mel-86a, murine fibrosarcoma MC57G and primary melanoma cell line Ma-Mel-51 were seeded out (100.00 cells/well) and infected with MOI=0.1 of LCMV strains WE, WE-Cl13(L) or P52-Cl13(L). After 20 h, cells were intracellularly stained for LCMV nucleoprotein (NP, clone VL4). The fluorescent intensity of LCMV infected cells was analyzed via flow cytometry to investigate replication capacity in the cells (FIG. 22).

Thereby it was proven that LCMV strain P52-Cl13(L) shows enhanced replication in human and murine tumor cells.

### Example 23

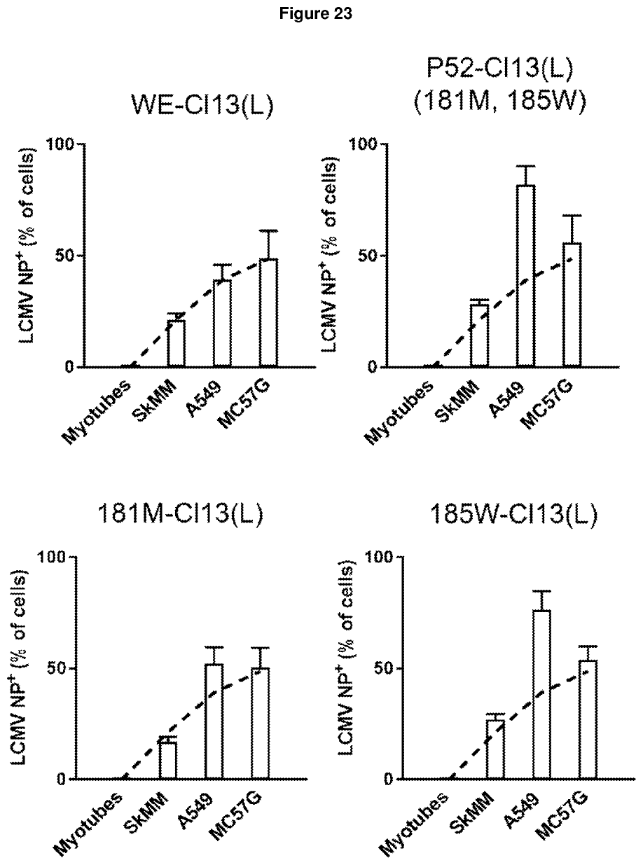

Differentiated human muscle myoblasts, human skeletal muscle myoblasts (SkMM), lung adenocarcinoma cell line A549 and murine fibrosarcoma cell line MC57G were seeded out (100.000 cell/well) and infected with MOI=0.1 of LCMV strains WE, WE-Cl13(L), P52-Cl13(L), I181M-Cl13(L) or R185W-Cl13(L). After 20 h, cells were intracellularly stained for LCMV nucleoprotein (NP, clone VL4). The percentage of LCMV infected cells (LCMV NP⁺) was analyzed via flow cytometry (FIG. 23).

Thereby it was shown that the mutations in the LCMV glycoprotein in positions 181 and 185 enhance the replication and infectivity in human and murine cancer cells but do not increase the tropism towards healthy human muscle cells.

### Example 24

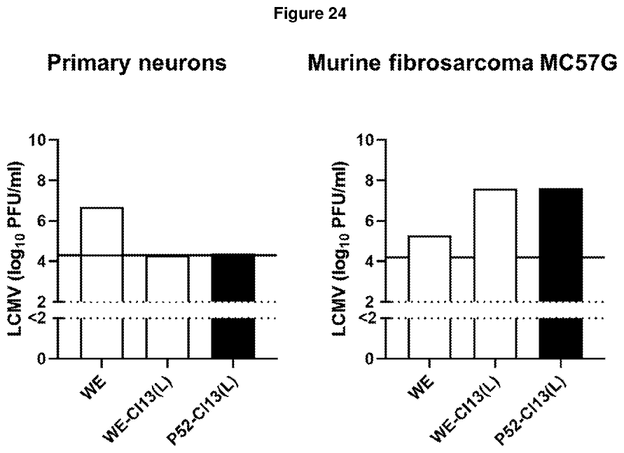

Primary human neurons and murine fibrosarcoma MC57G cells were tested for their LCMV replication potential. 100.000 cells per well were seeded out and infected with MOI=0.1 of LCMV strains WE, WE-Cl13(L) or P52-Cl13(L). Supernatant was harvested 48 h after infection. Virus titers were analyzed by focus forming assay (FIG. 24).

Thereby it was proven that reassortant LCMV strains WE-Cl13(L) and P52-Cl13(L) do not show tropism towards healthy human neurons but show enhanced replication in murine cancer cells compared to wild type LCMV strain WE.

### Example 25

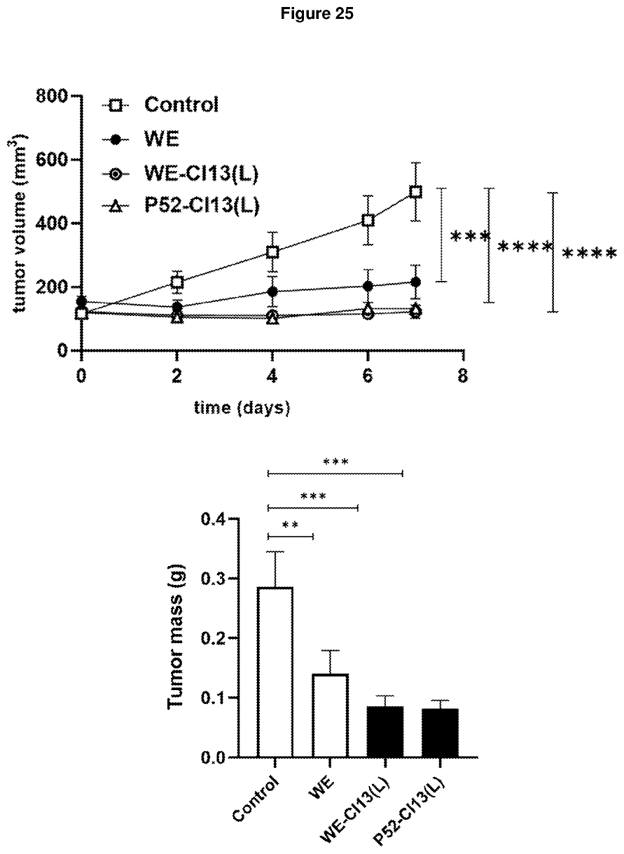

C57BL/6 mice were subcutaneously treated with murine melanoma B16 cells and intravenously infected with 2×10⁴ PFU of LCMV strains WE, WE-Cl13(L) or P52-Cl13(L) or were control treated (n=5 animals per group). Tumor growth was measured over seven days. Tumor mass was measured at finalization (d7) (FIG. 25).

Thereby it was proven that LCMV strains WE-CL13(L) and P52-Cl13(L) show an increased antitumoral effect compared to wild type LCMV strain WE.

### Example 26

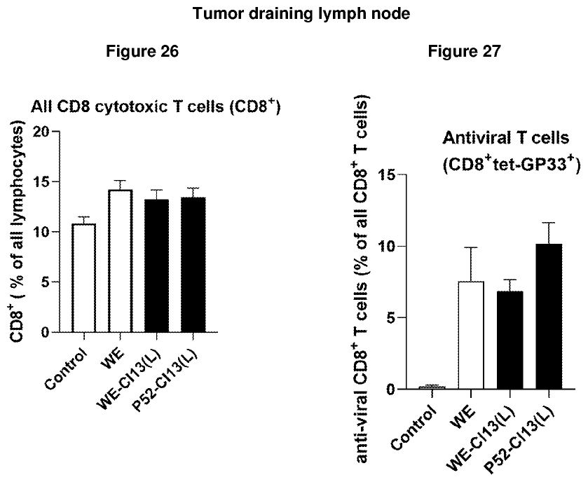

C57BL/6 mice were subcutaneously treated with B16 melanoma cells and intravenously infected with 2×10⁴ PFU of LCMV strains WE, WE-Cl13(L) or P52-Cl13(L) or were control treated (n=5 animals per group). Mice were sacrificed on day seven after infection. Tumor draining lymph node resident cytotoxic CD8⁺ T cells were stained via fluorescent labelled anti-CD8 antibody (eBioscience) and analyzed via flow cytometry (FIG. 26).

Thereby it was proven that LCMV strains WE-CL13(L) and P52-Cl13(L) enhance cytotoxic T cell accumulation in tumor draining lymph nodes.

### Example 27

C57BL/6 mice were subcutaneously treated with B16 melanoma cells and intravenously infected with 2×10⁴ PFU of LCMV strains WE, WE-Cl13(L) or P52-Cl13(L) or were control treated (n=5 animals per group). Mice were sacrificed on day seven after infection. Tumor draining lymph node resident cytotoxic, antiviral CD8⁺ GP33-tetramer positive T cells were stained via fluorescent labeled antibodies (eBioscience, NIH) and analyzed via flow cytometry on day seven after infection (FIG. 27).

Thereby it was proven that LCMV strain P52-Cl13(L) increases accumulation of virus specific T cells in tumor draining lymph nodes.

### Example 28

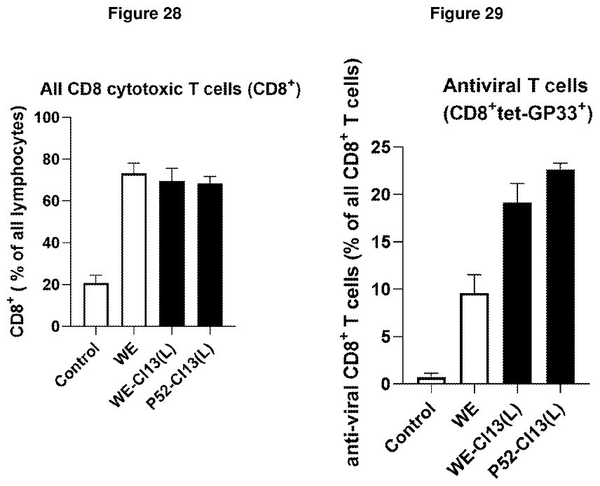

C57BL/6 mice were subcutaneously treated with B16 melanoma cells and intravenously infected with 2×10⁴ PFU of LCMV strains WE, WE-Cl13(L) or P52-Cl13(L) or were control treated (n=5 animals per group). Mice were sacrificed on day seven after infection. Tumor resident cytotoxic CD8⁺ T cells were stained via fluorescent labelled anti-CD8 antibody (eBioscience) and analyzed via flow cytometry (FIG. 28).

Thereby it was proven that LCMV strains WE-CL13(L) and P52-Cl13(L) enhance cytotoxic T cell accumulation in tumor.

### Example 29

C57BL/6 mice were subcutaneously treated with B16 melanoma cells and intravenously infected with 2×10⁴ PFU of LCMV strains WE, WE-Cl13(L) or P52-Cl13(L) or were control treated (n=5 animals per group). Tumor draining lymph node resident cytotoxic, antiviral CD8⁺ GP33-tetramer positive T cells were stained via fluorescent labeled antibodies (eBioscience, NIH) and analyzed via flow cytometry on day seven after infection (FIG. 29).

Thereby it was proven that treatment with LCMV strain P52-Cl13(L) strongly enhances antiviral T cell accumulation in tumor tissue.

### Example 30

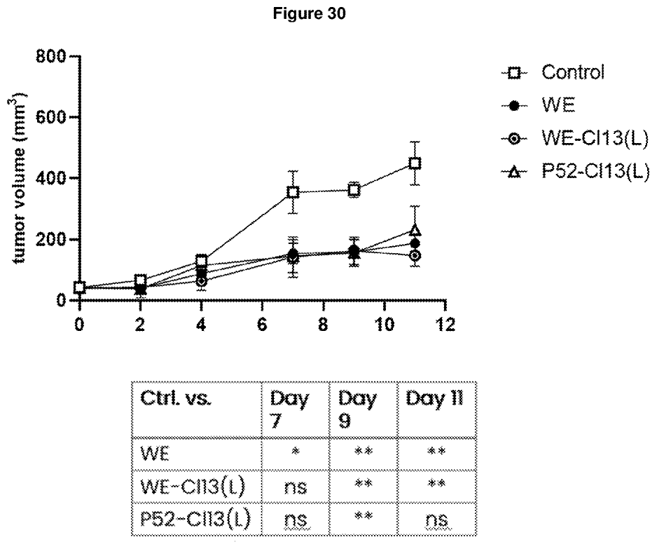

C57BL/6 mice were subcutaneously treated with murine MC38 colon carcinoma cells. After visible tumor formation mice were intravenously treated with 2×10⁴ PFU of LCMV strains WE, WE-Cl13(L), P52-Cl13(L) or control treated (n=4). Tumor volume was measured until eleven days after infection (FIG. 30).

Thereby it was proven that LCMV strain P52-Cl13(L) induces strong tumor growth inhibition.

### Example 31

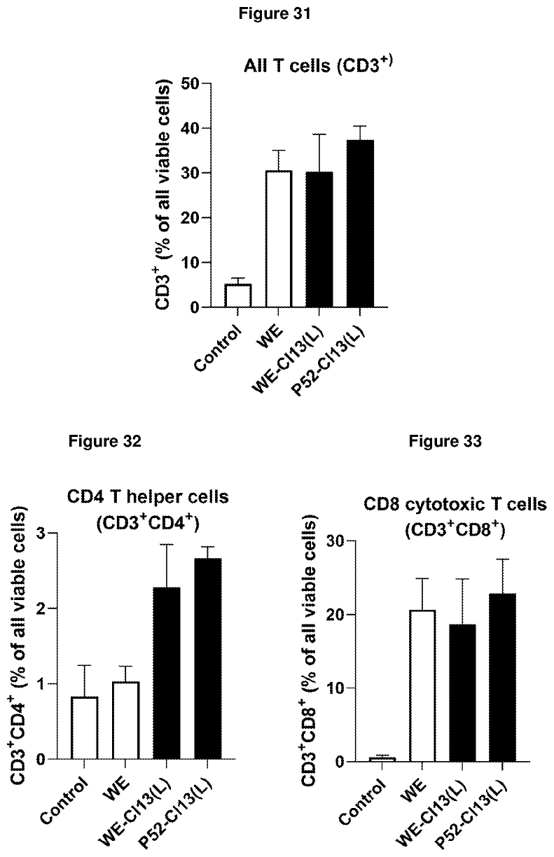

C57BL/6 mice were subcutaneously treated with murine MC38 colon carcinoma cells. After visible tumor formation mice were intravenously treated with 2×10⁴ PFU of LCMV strains WE, WE-Cl13(L), P52-Cl13(L) or control treated (n=4). Tumors were dissected on day eleven. T cells were stained with fluorescent anti-CD3 antibody (eBioscience) and analyzed via flow cytometry (FIG. 31).

Thereby it was proven that LCMV strain P52-Cl13(L) shows a clear tendency towards an enhanced T cell accumulation in tumor tissue compared to LCMV strains WE, WE-Cl13(L) and control treated tumors.

### Example 32

C57BL/6 mice were subcutaneously treated with murine MC38 colon carcinoma cells. After visible tumor formation mice were intravenously treated with 2×10⁴ PFU of LCMV strains WE, WE-Cl13(L), P52-Cl13(L) or control treated (n=4). Tumors were dissected on day eleven. T helper cells were stained with fluorescent anti-CD3 and anti-CD4 antibodies (eBioscience) and analyzed via flow cytometry (FIG. 32).

Thereby it was shown that LCMV strains WE-Cl13(L) and P52-Cl13(L) induce a stronger T helper cell infiltration into tumor tissue compared to tumors treated with wildtype LCMV strain WE or control treated.

### Example 33

C57BL/6 mice were subcutaneously treated with murine MC38 colon carcinoma cells. After visible tumor formation mice were intravenously treated with 2×10⁴ PFU of LCMV strains WE, WE-Cl13(L), P52-Cl13(L) or control treated (n=4). Tumors were dissected on day eleven. Cytotoxic T cells were stained with fluorescent anti-CD3 and anti-CD8 antibodies (eBioscience) and analyzed via flow cytometry (FIG. 33).

Thereby it was proven that LCMV strain P52-Cl13(L) enhances cytotoxic T cell accumulation in tumor compared to control treatment.

### Example 34

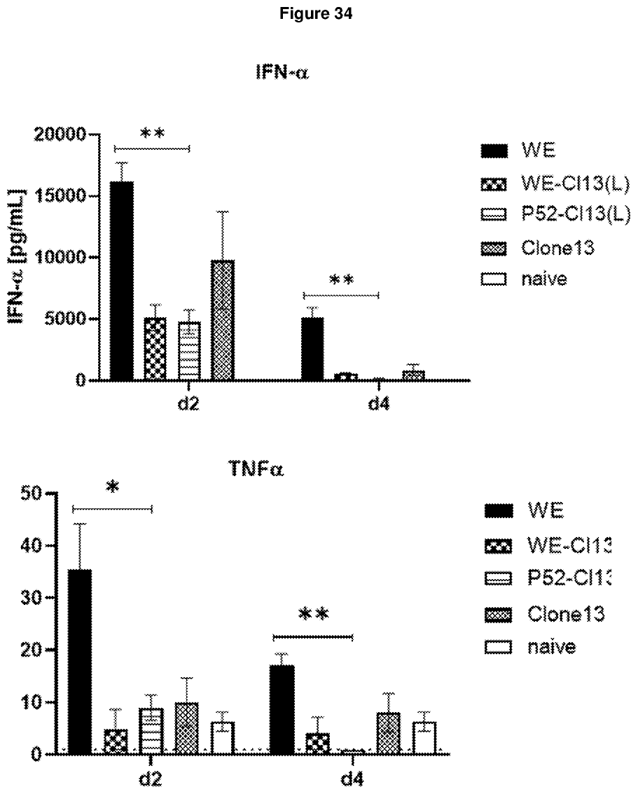

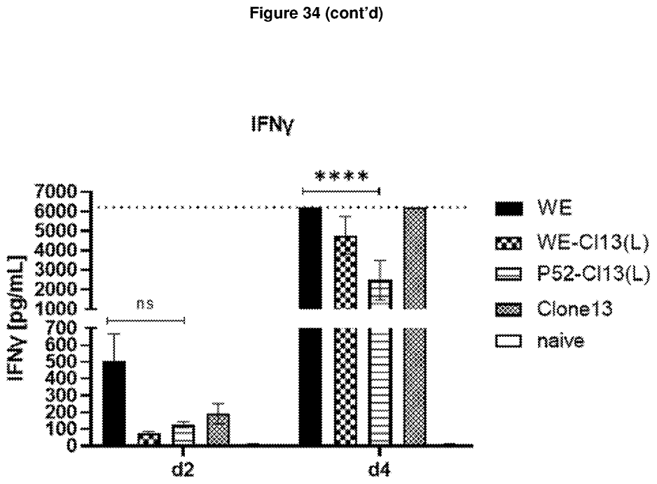

C57BL/6 mice were intravenously infected with 2×10⁴ PFU per mouse of LCMV strains WE, WE-Cl13(L), P52-Cl13(L) or left untreated (n=3). Blood was taken on day two and day four after infection. Serum cytokine levels of IFNα, TNFα and IFNγ were analyzed by LegendPlex assay (BioLegend) (FIG. 34).

Thereby it was proven that LCMV reassortant strains WE-Cl13(L) and P52-Cl13(L) induce reduced systemic cytokine levels compared to wildtype LCMV strains WE and Clone13.

### Example 35

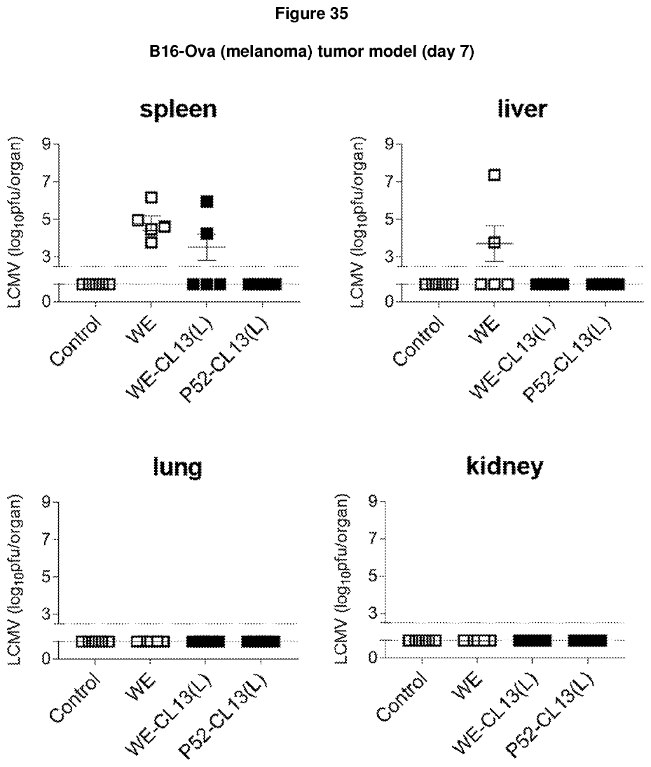

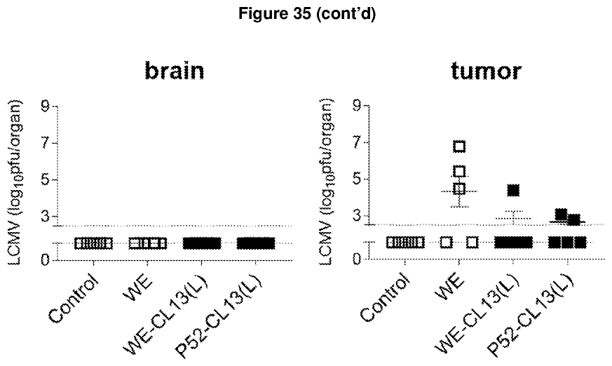

C57BL/6 mice were subcutaneously treated with B16-Ova melanoma cells and intravenously infected with 2×10⁴ PFU of LCMV strains WE, WE-Cl13(L) or P52-Cl13(L) or were control treated (n=5 animals per group). Mice were sacrificed at day seven post infection. Spleen, liver, lung, kidney brain and tumor tissue were analyzed for LCMV replication via focus forming assay (FIG. 35).

Thereby it was shown that LCMV reassortant P52-Cl13(L) displays a decreased replication in healthy organs by simultaneously showing measurable replication in tumor tissue.

### Example 36

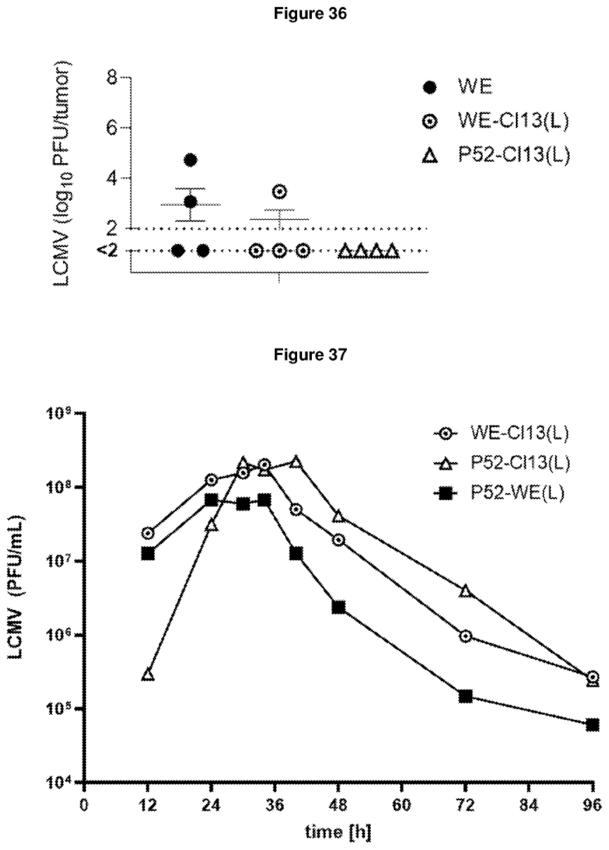

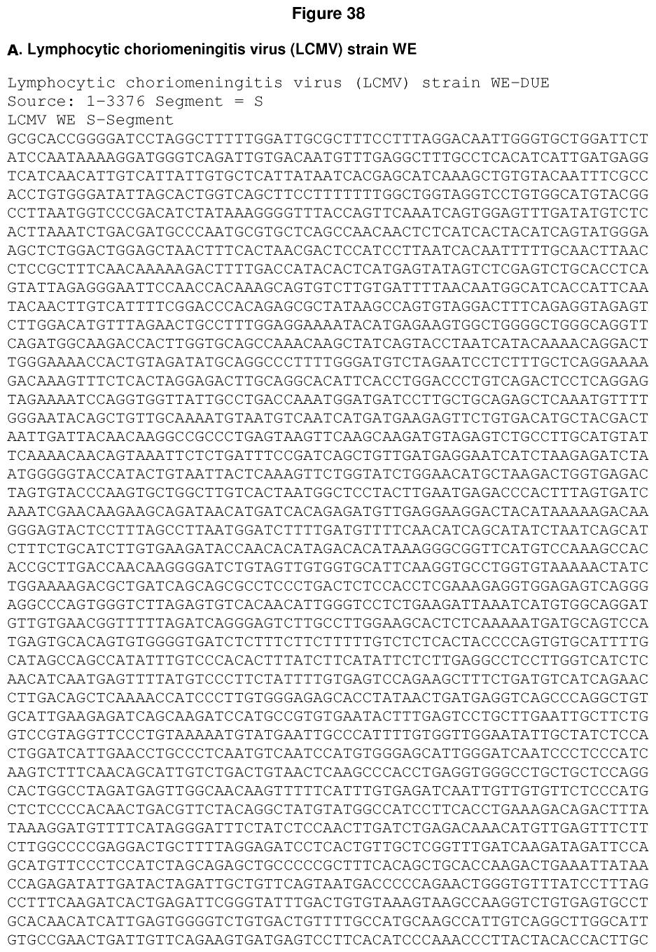

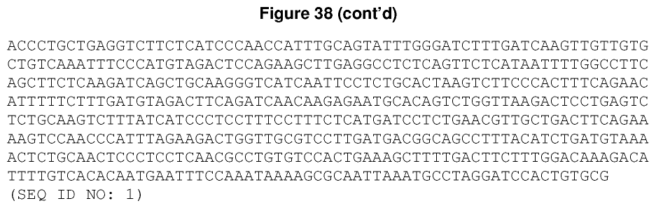

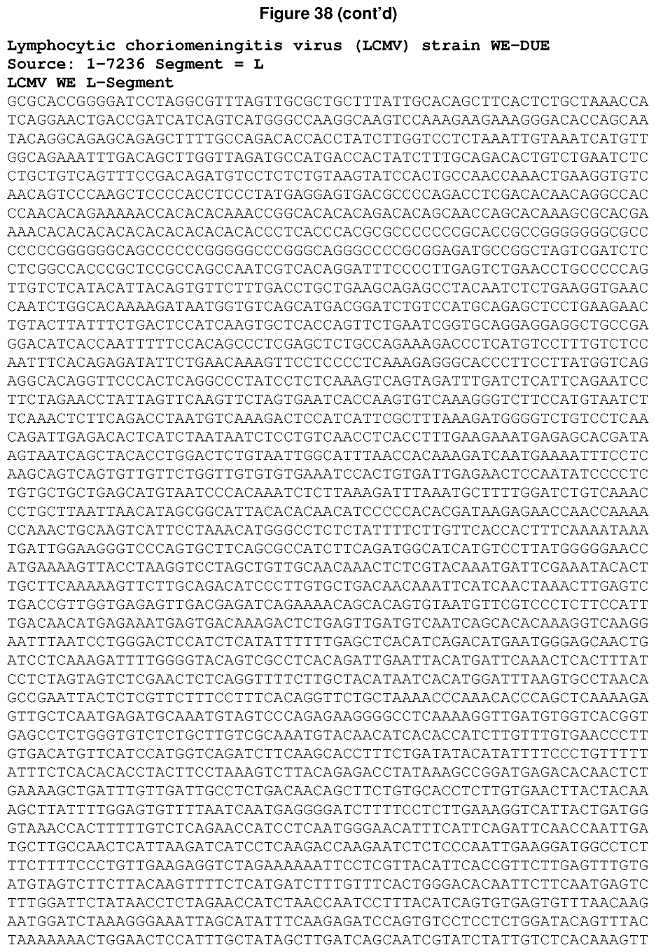

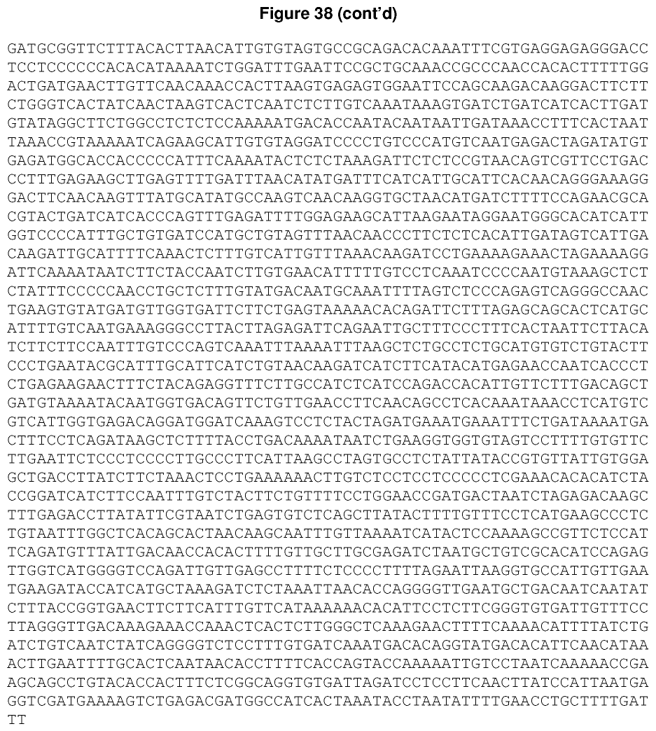

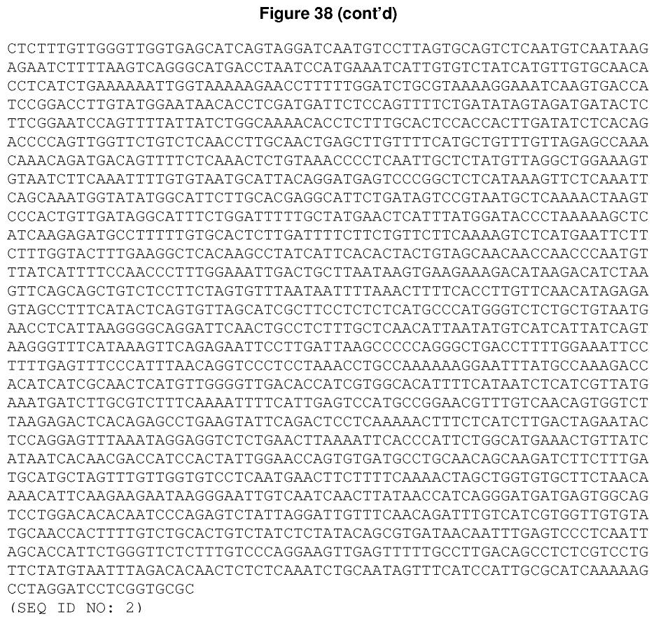

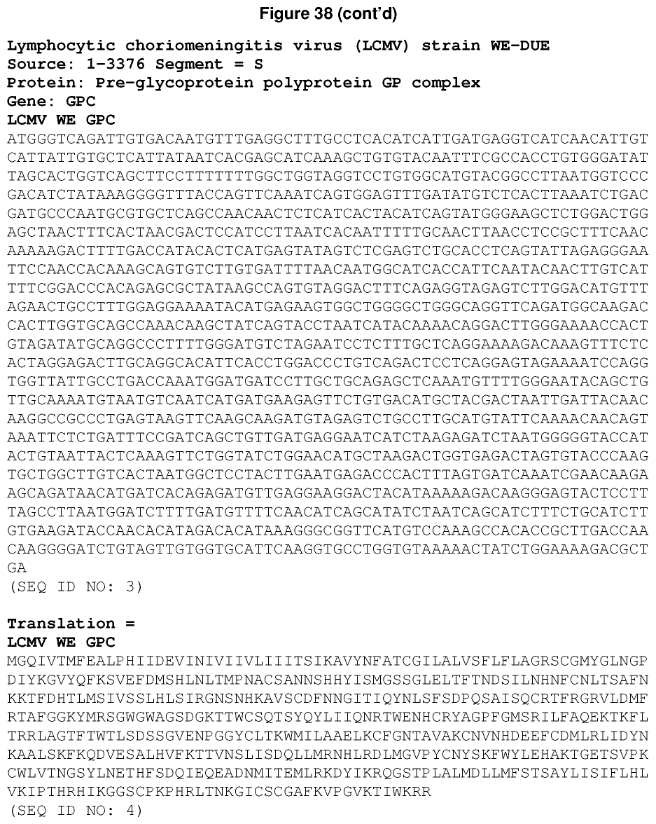

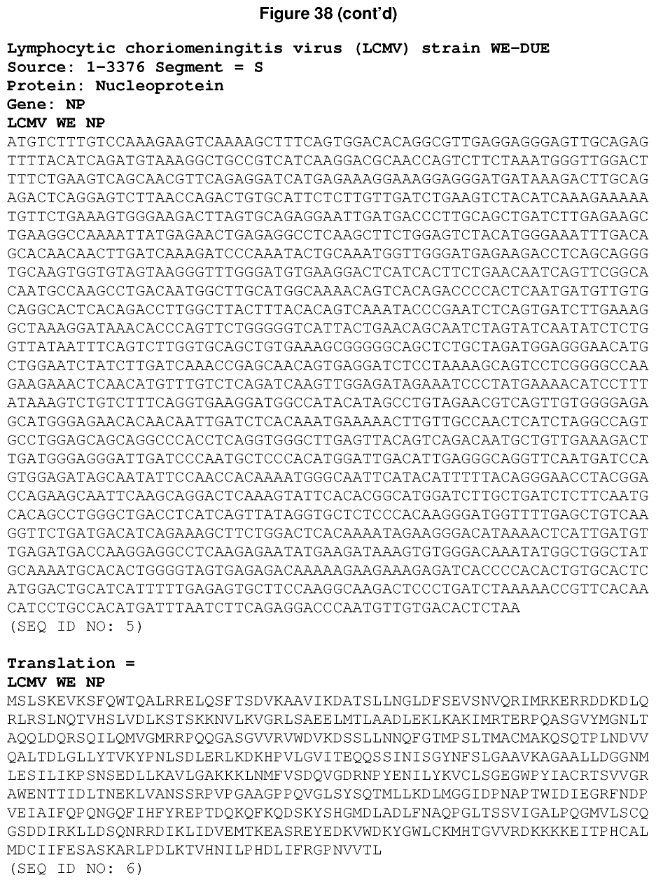

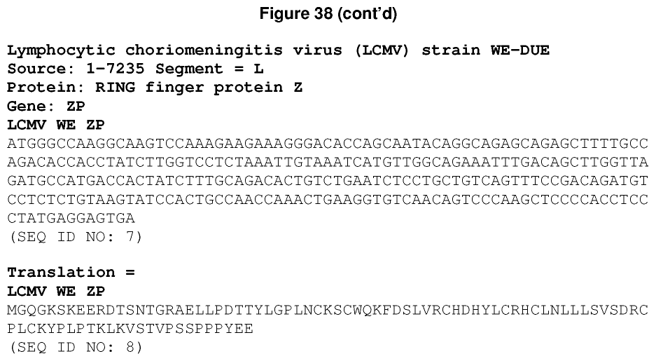

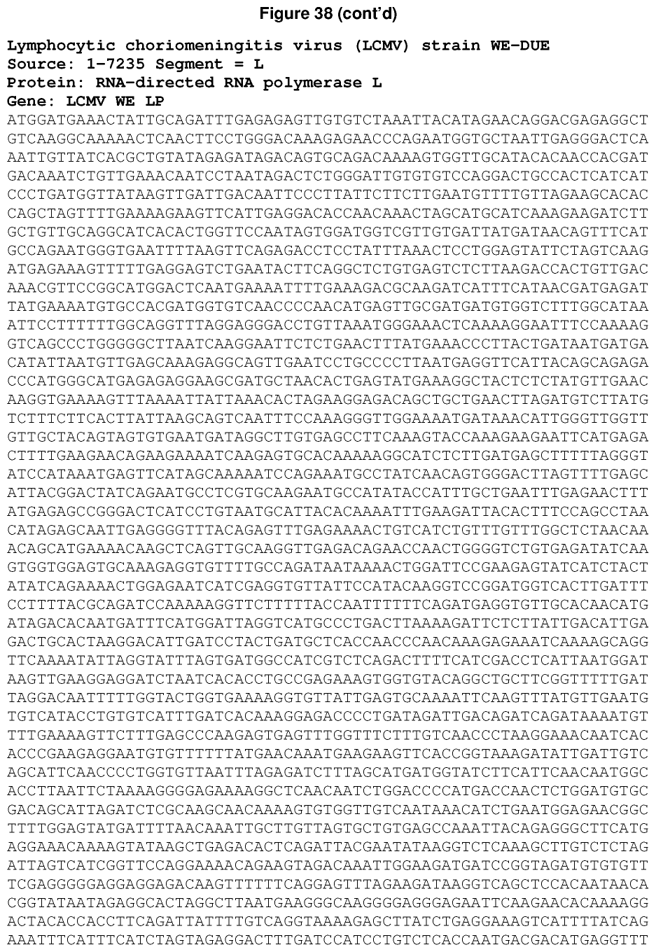

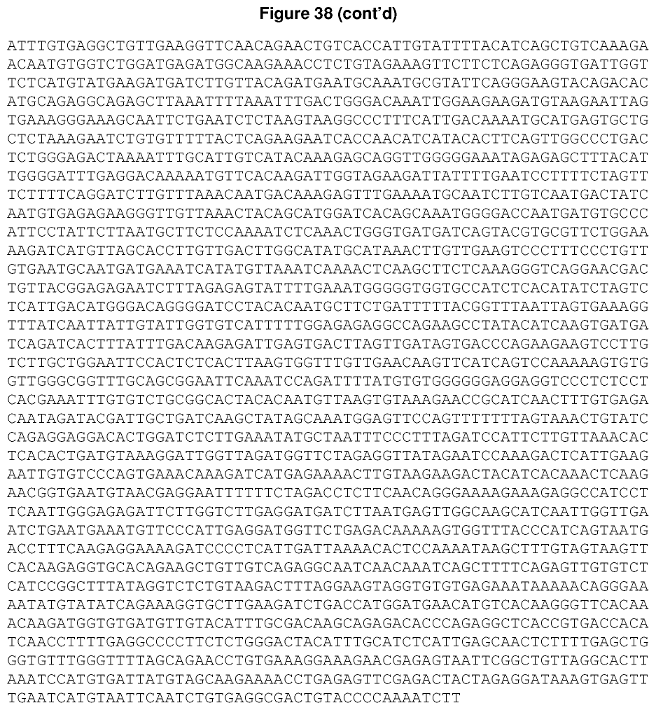

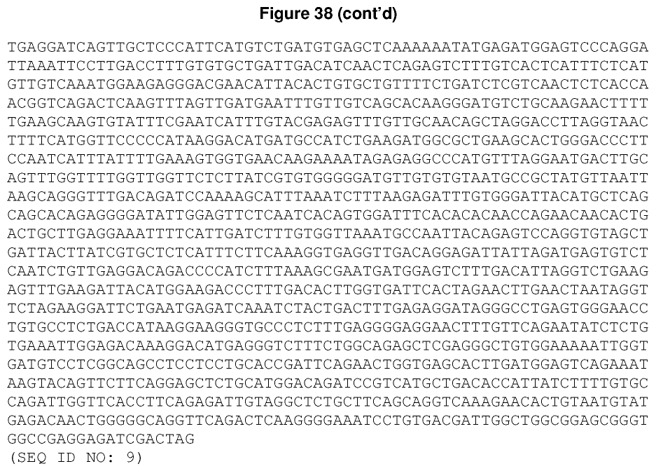

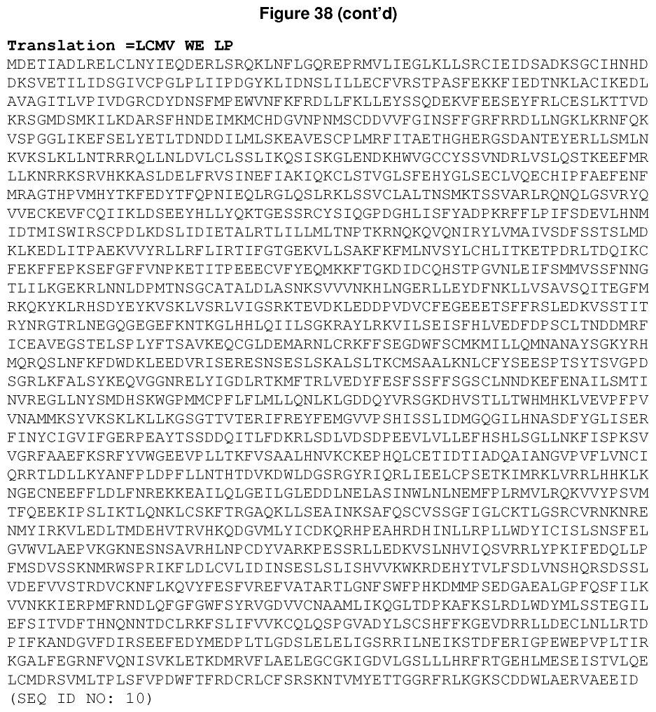

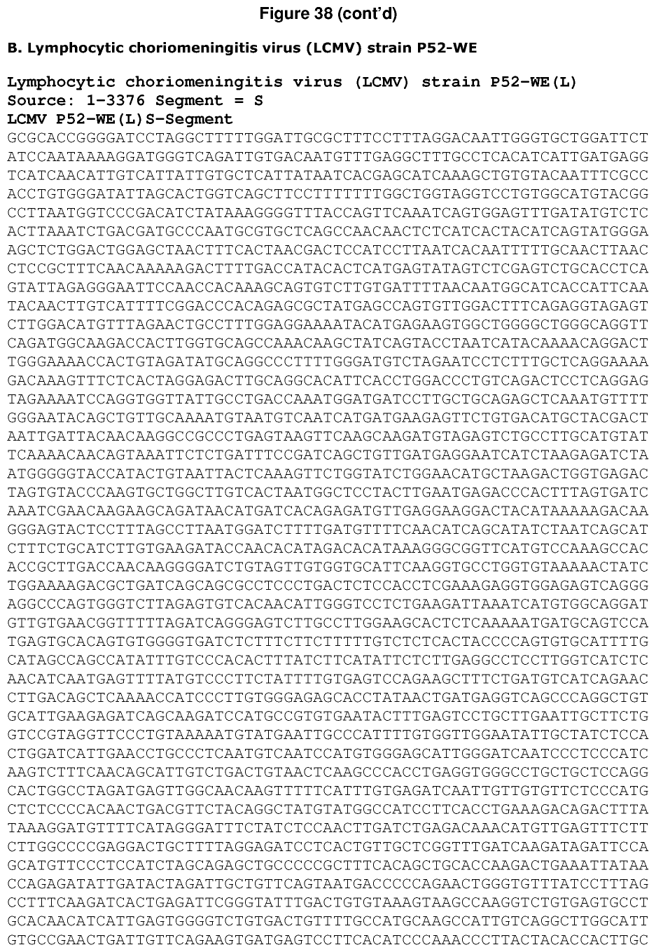

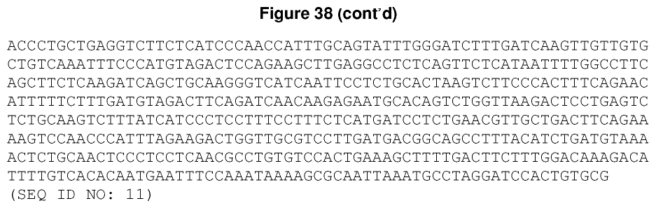

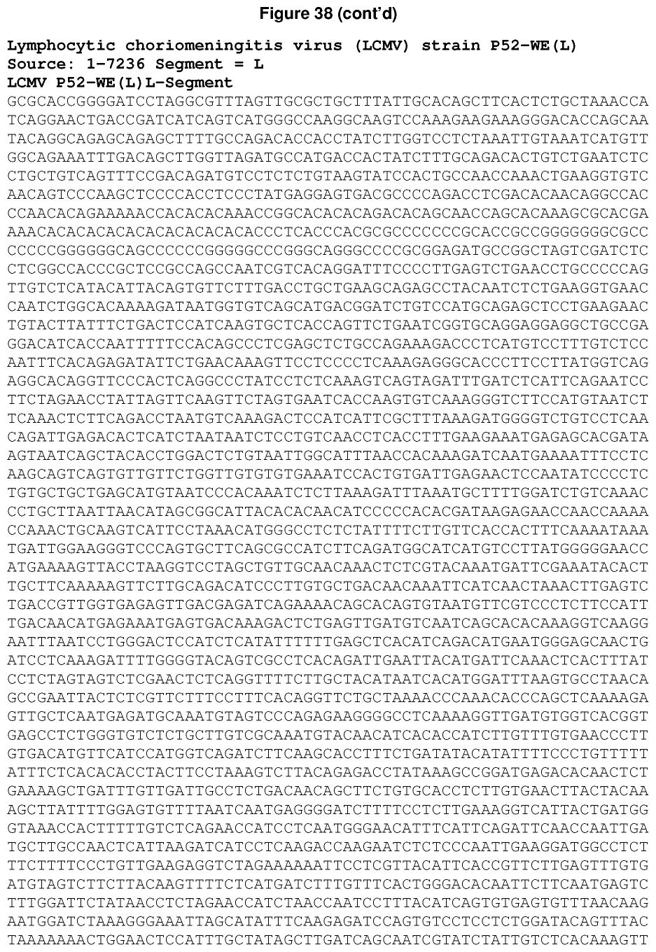

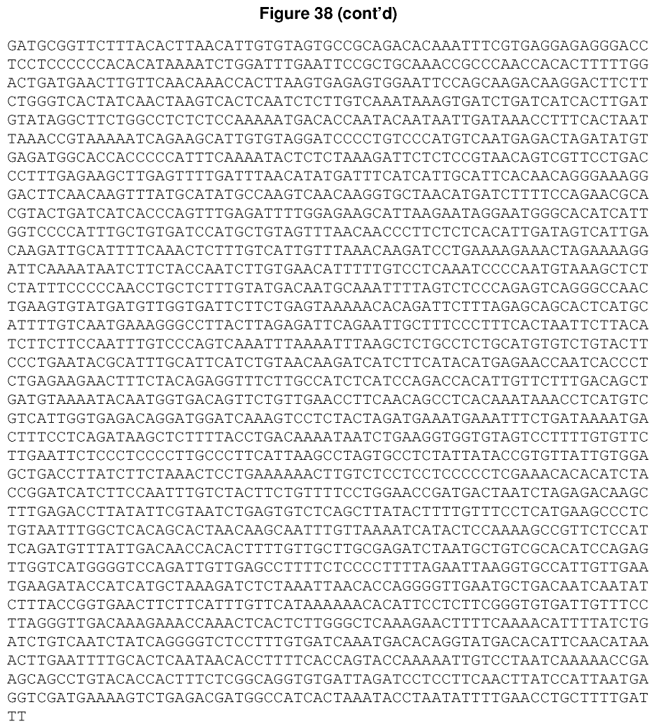

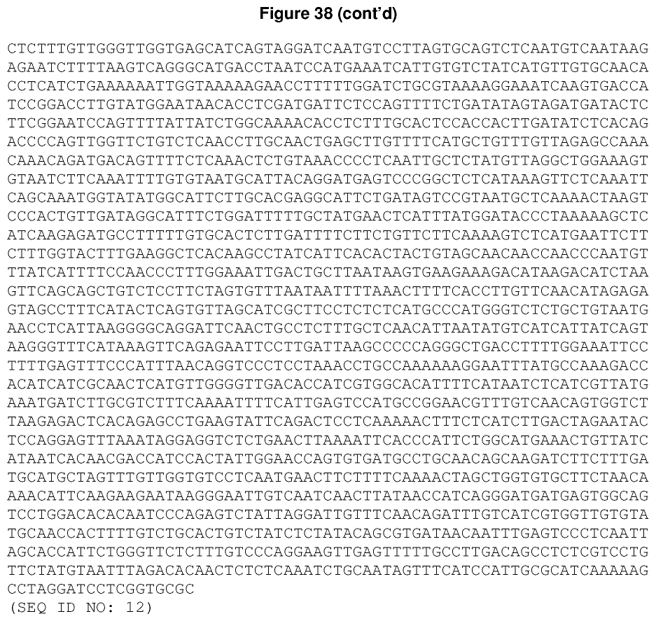

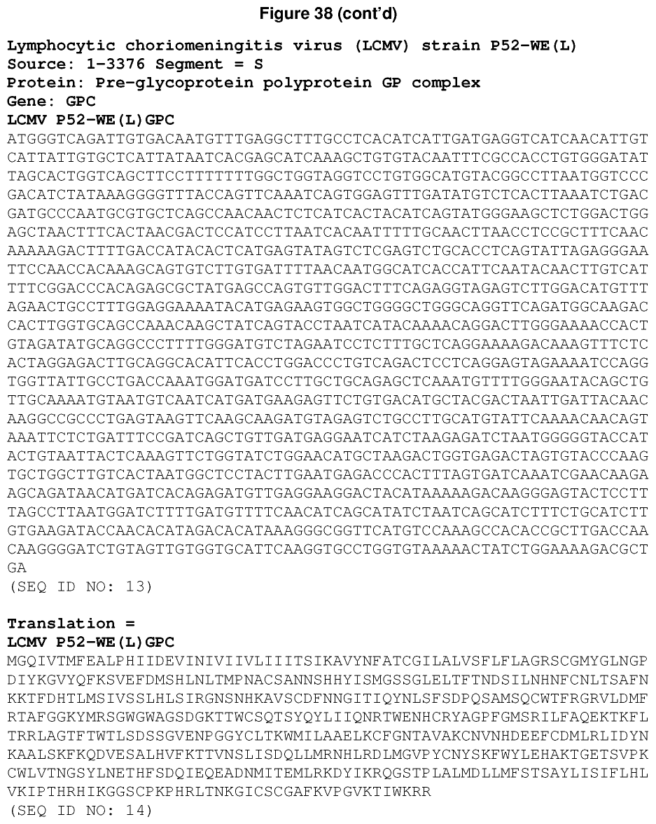

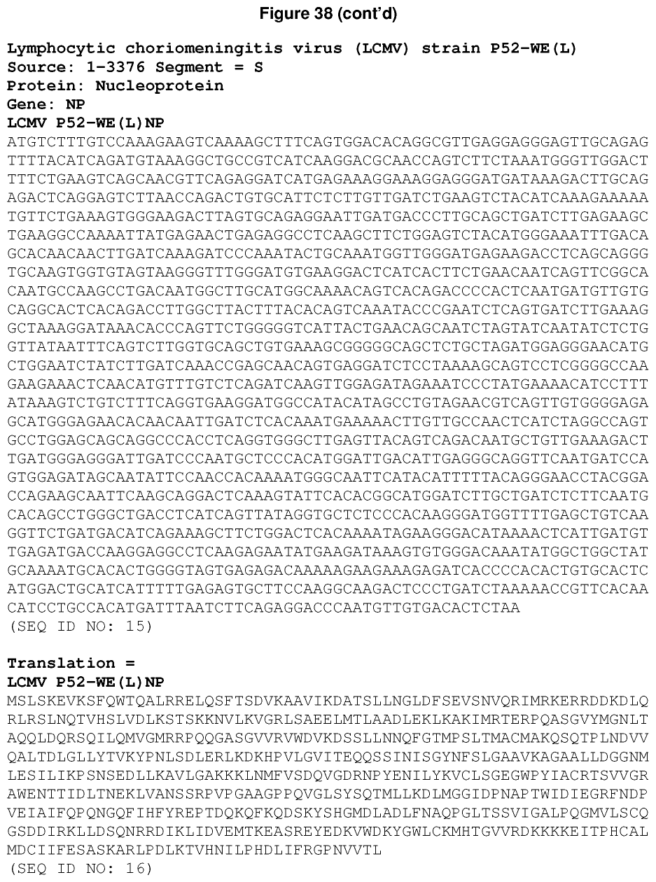

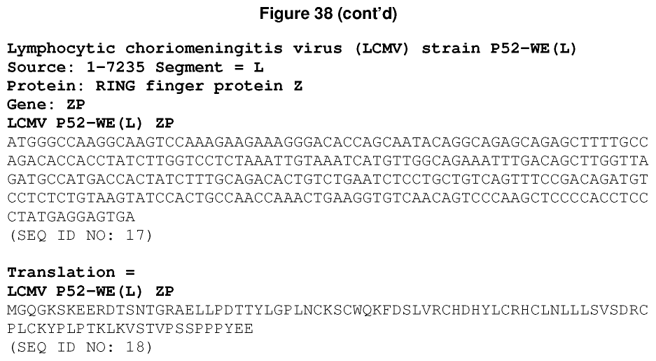

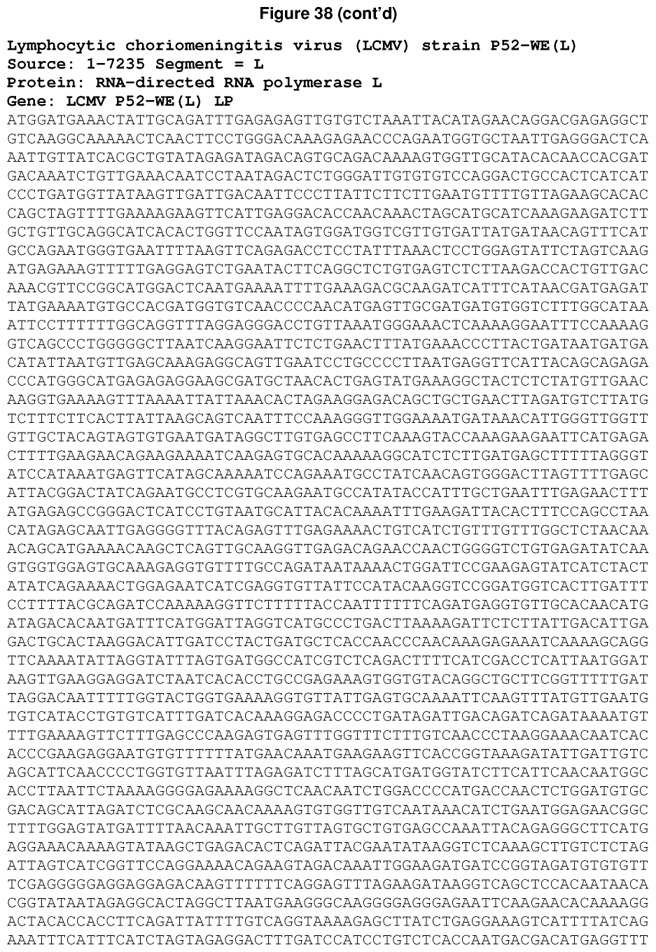

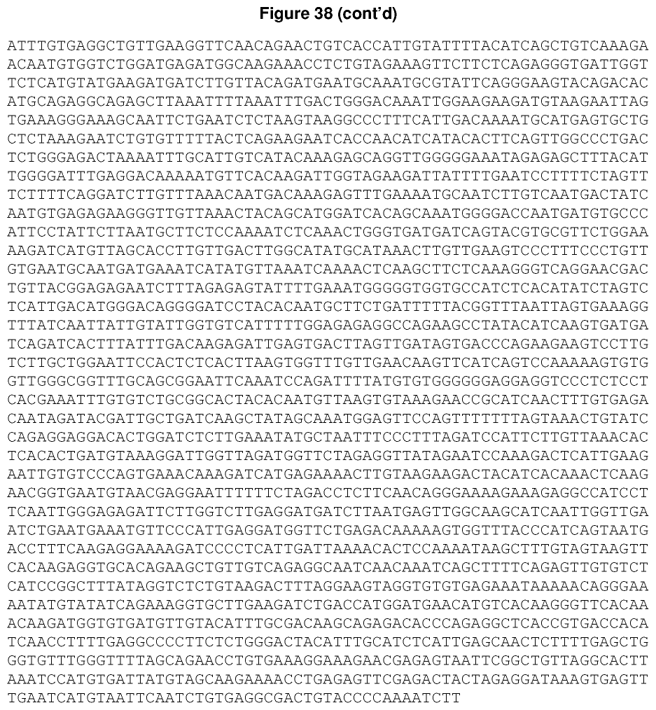

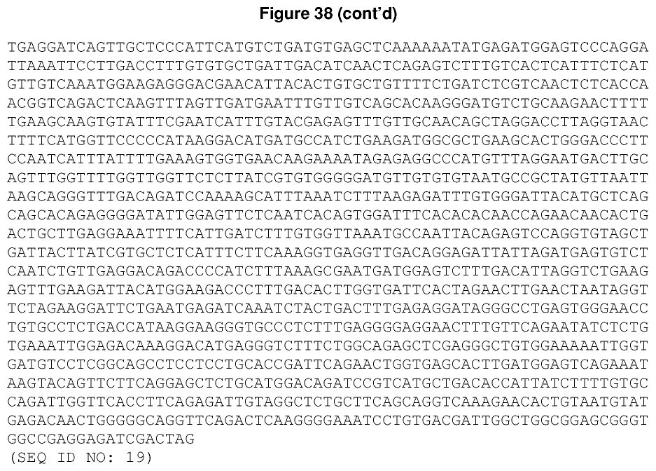

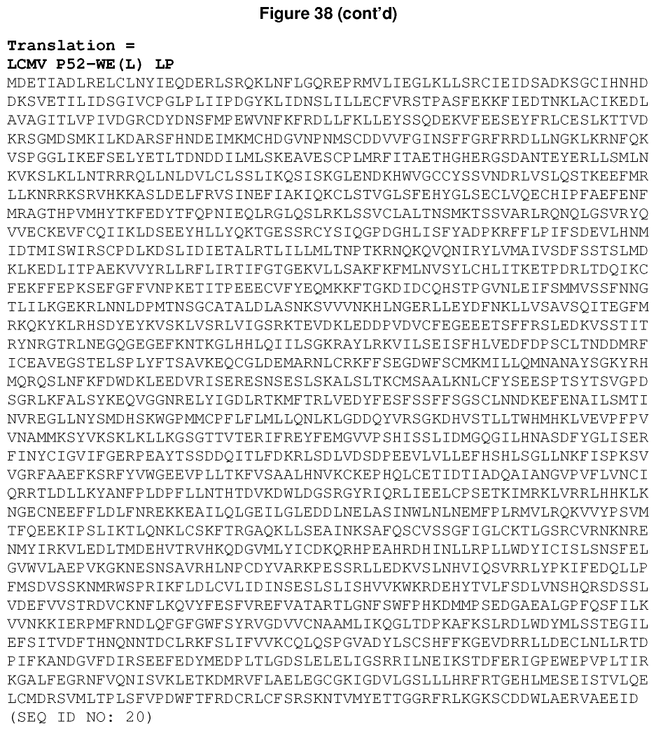

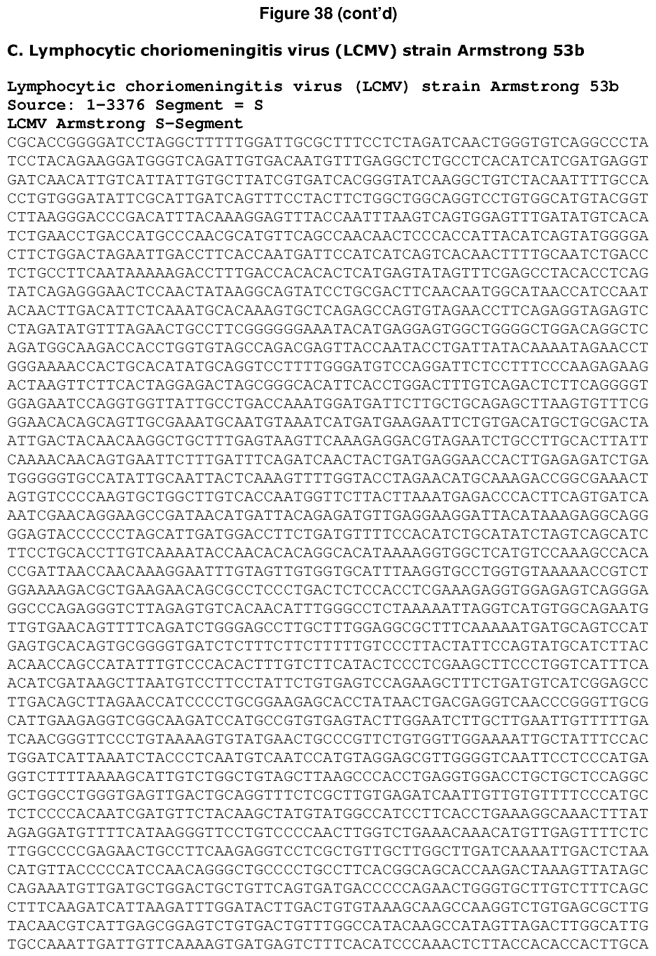

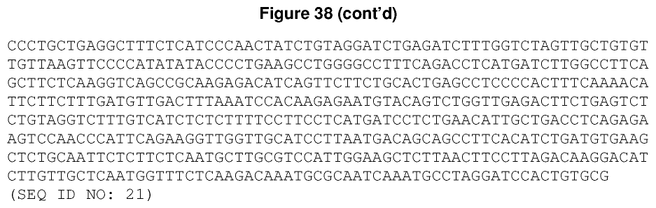

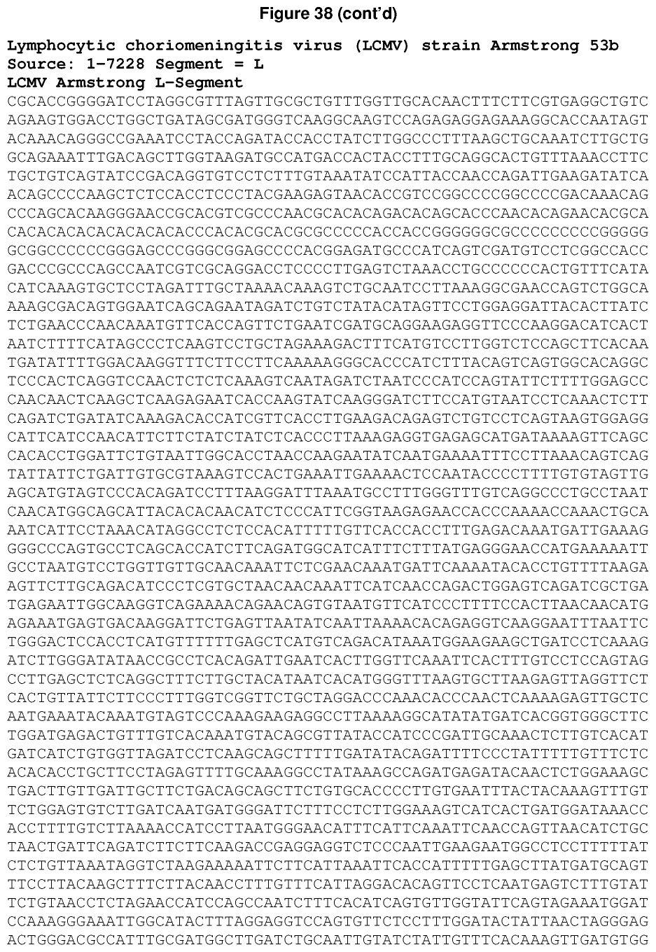

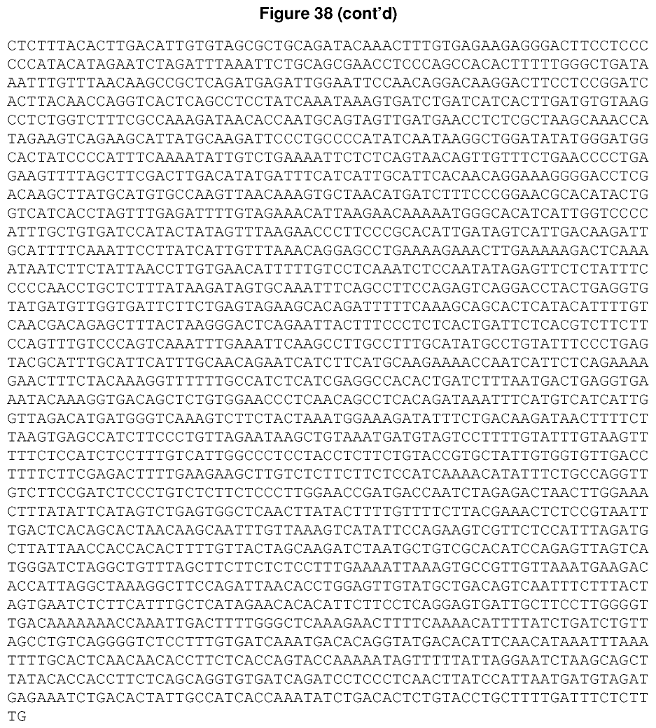

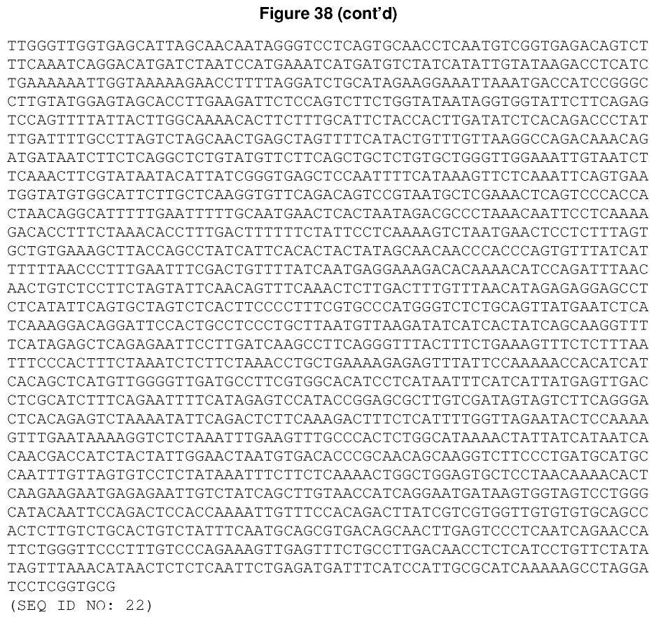

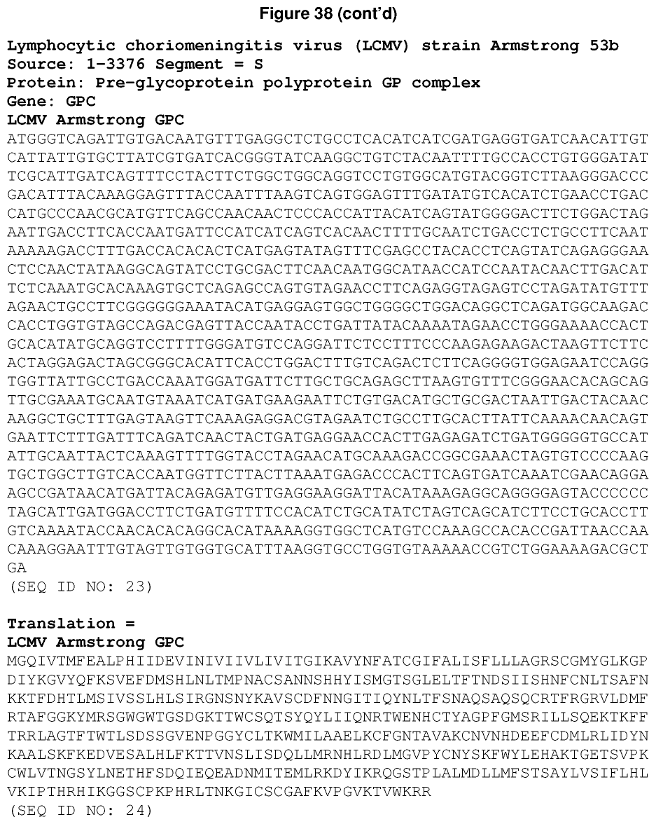

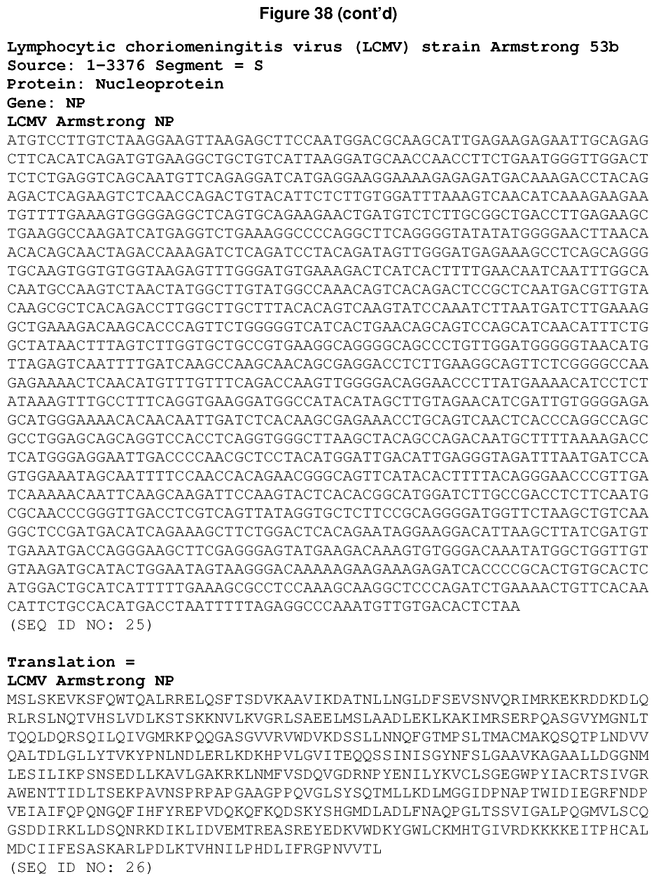

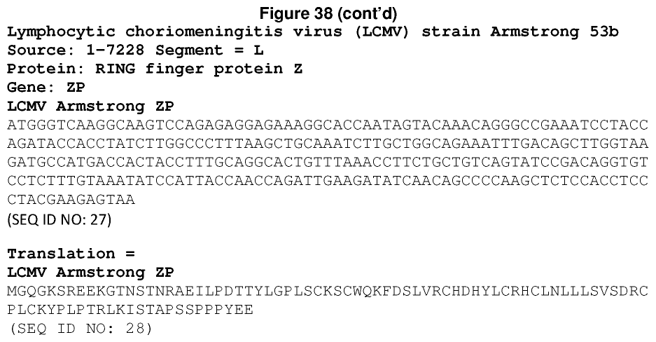

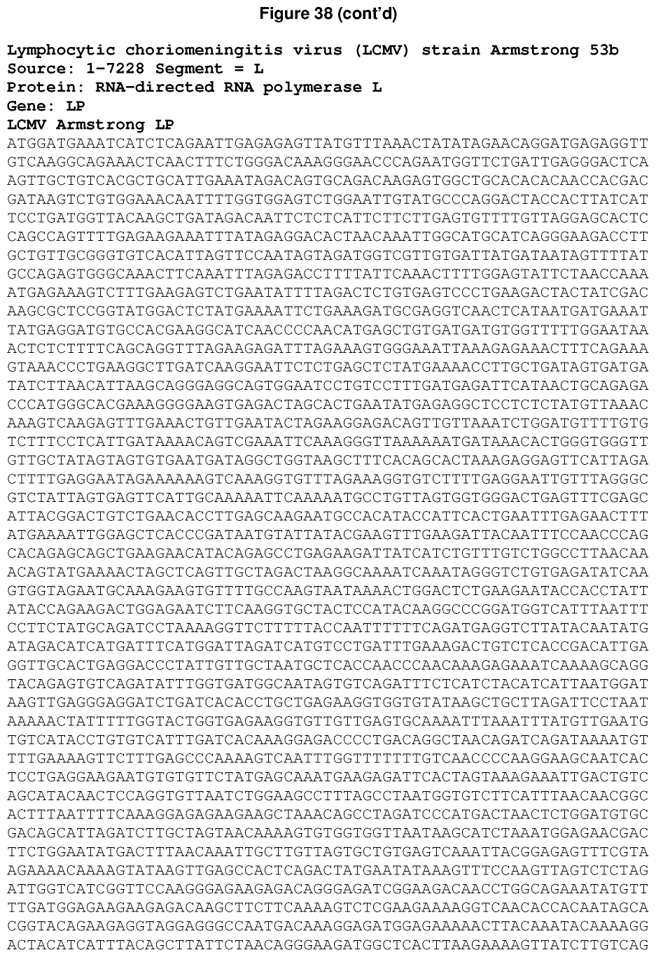

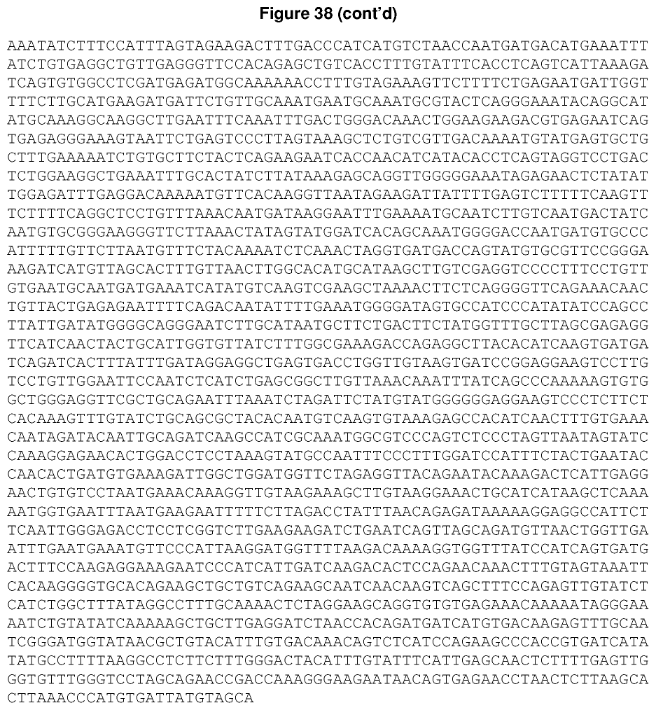

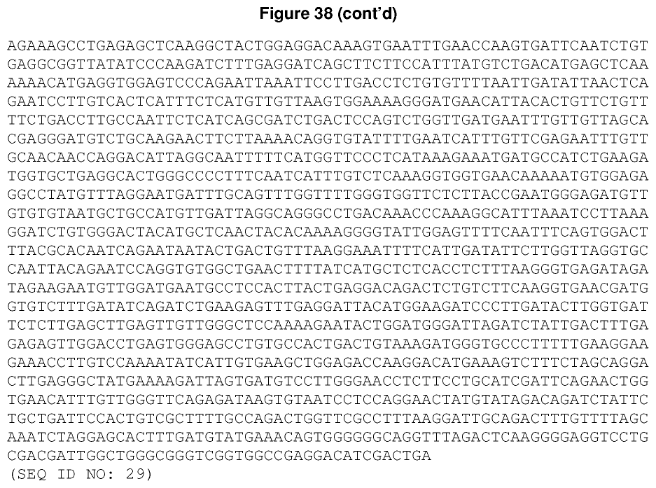

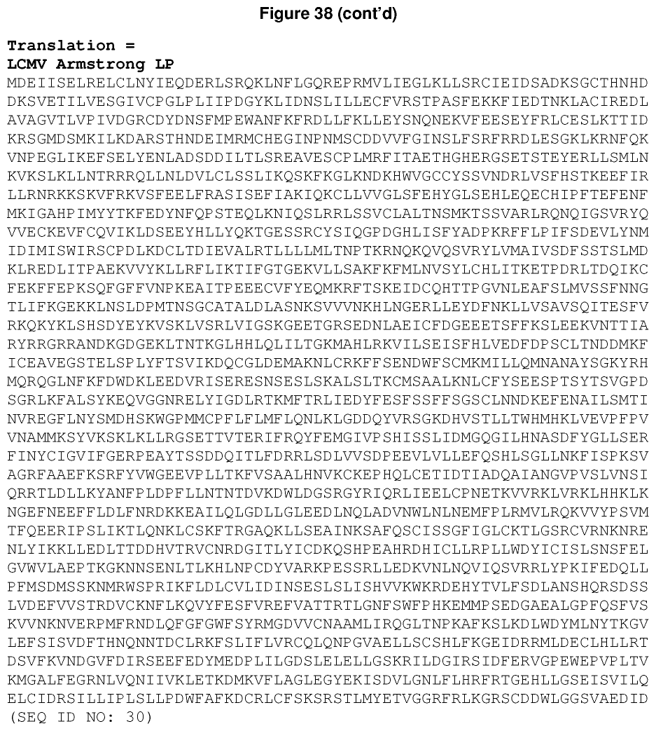

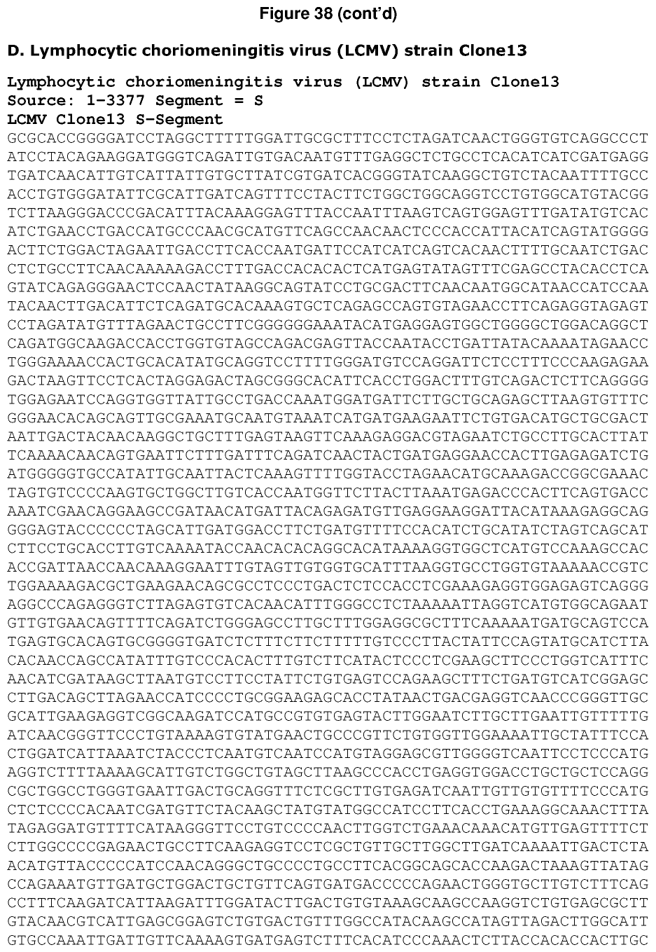

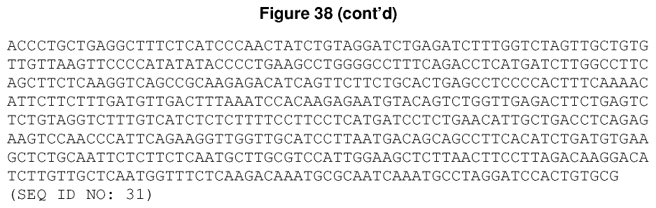

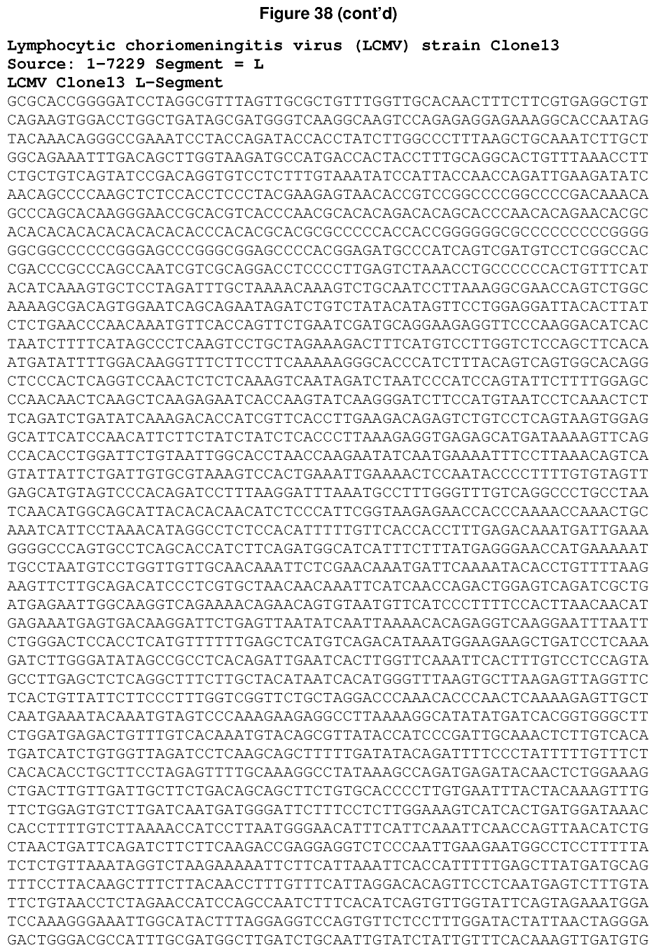

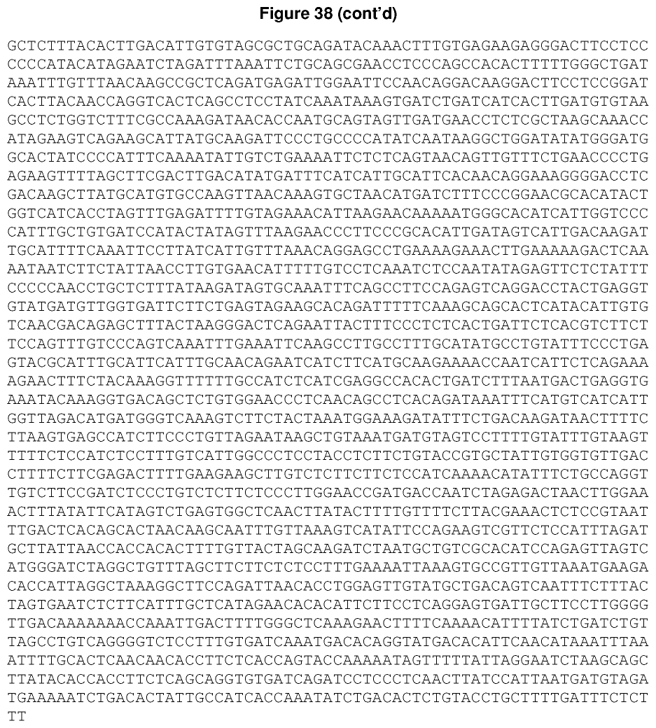

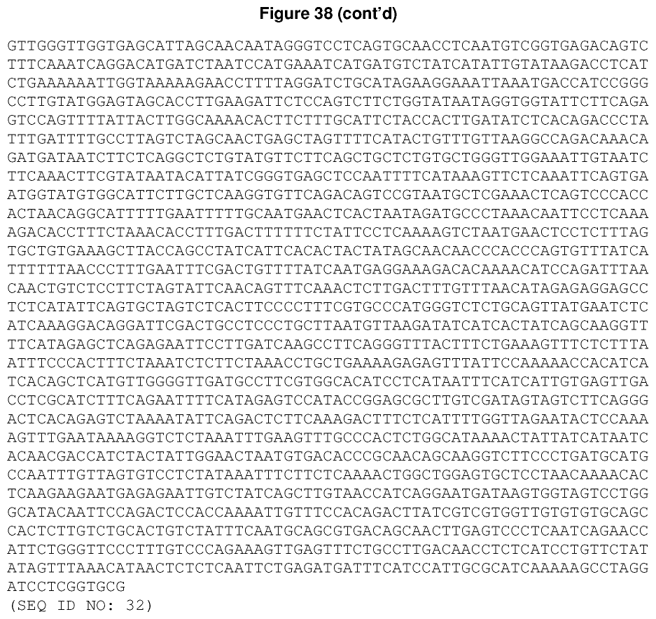

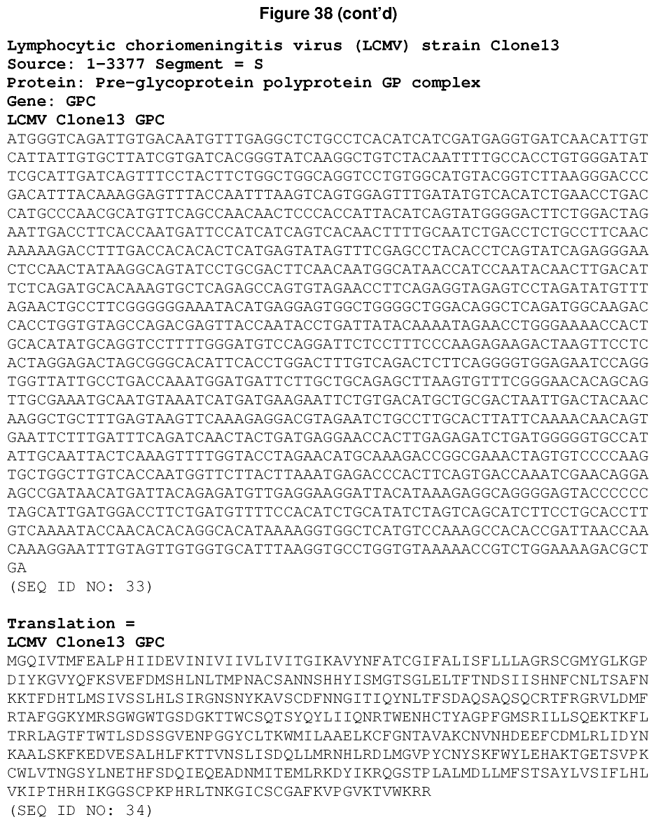

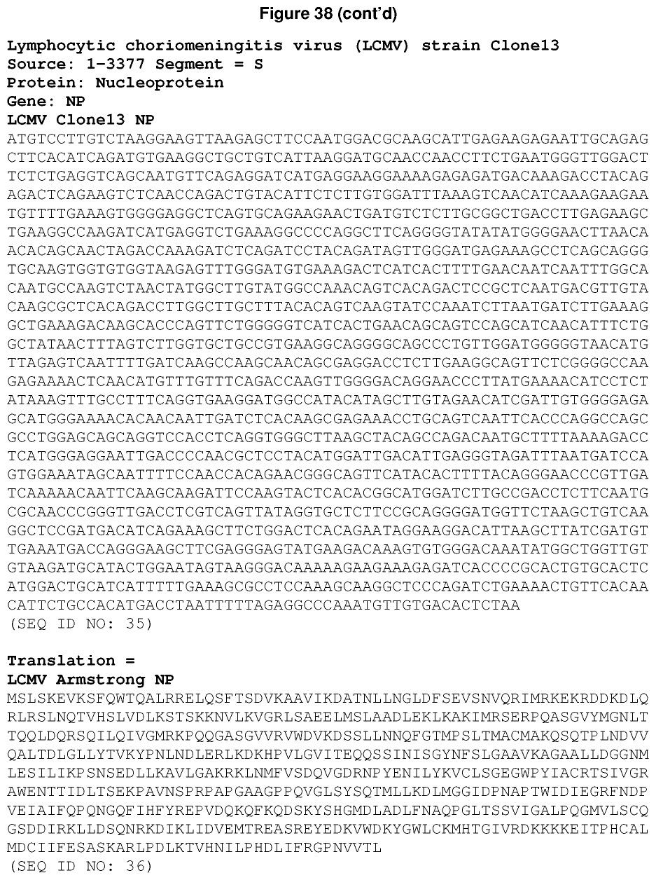

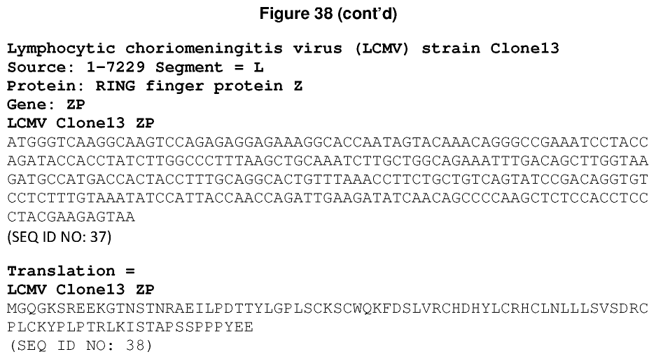

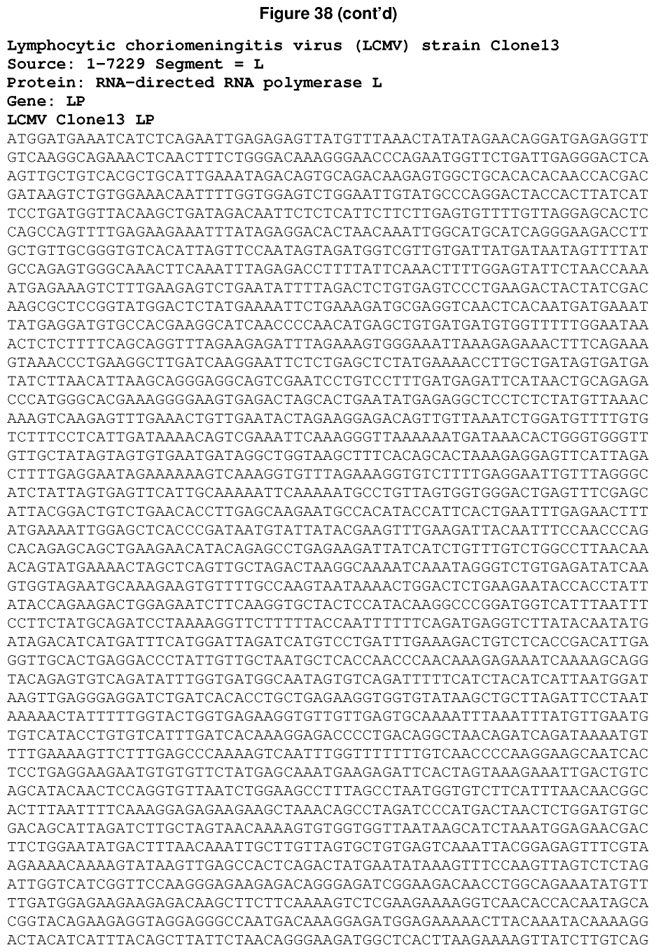

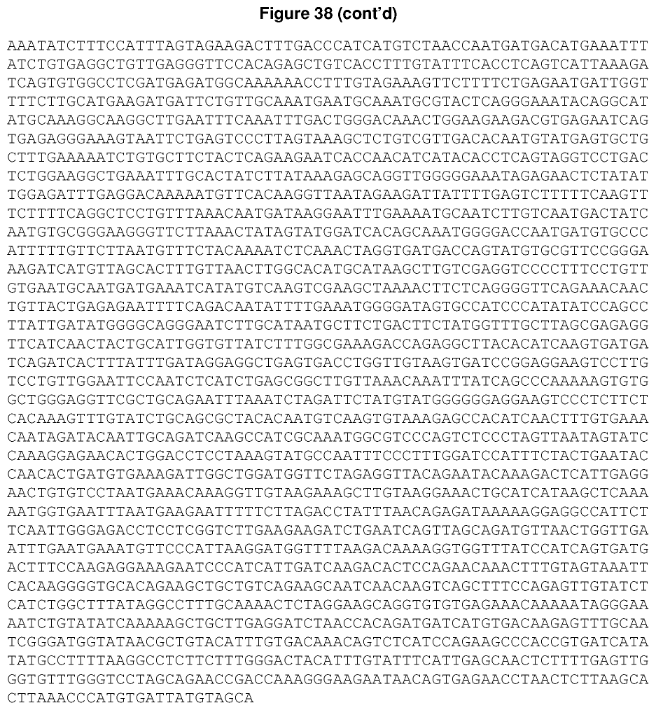

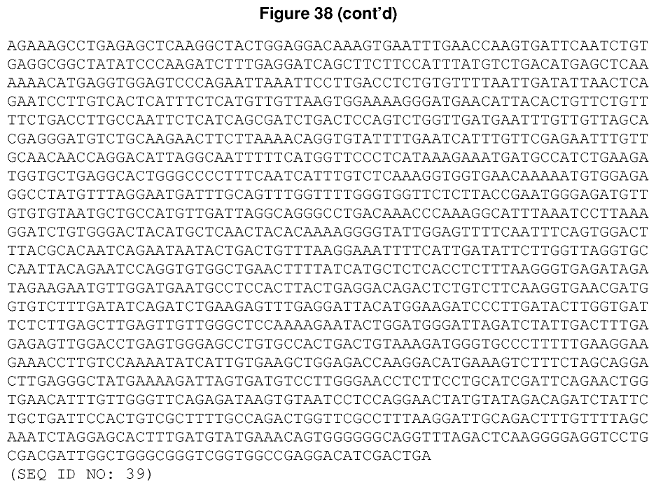

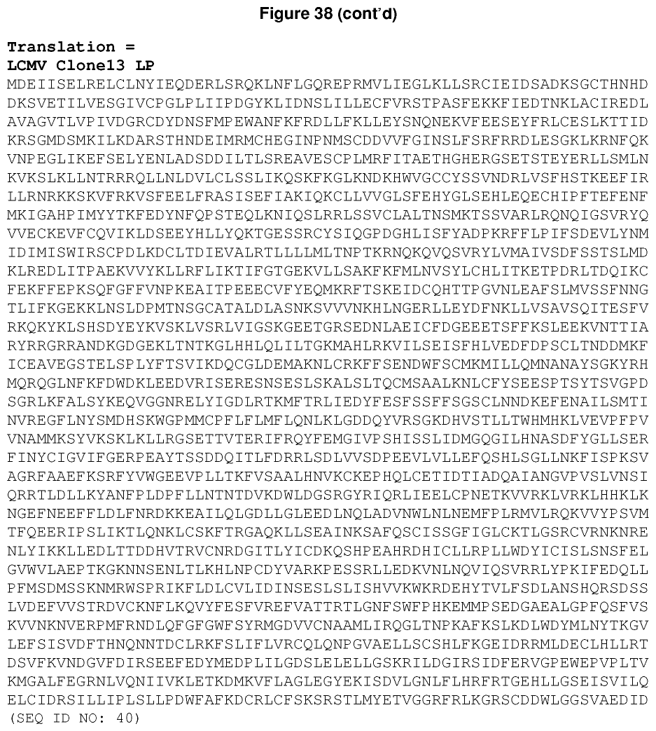

MC38 tumor cells were subcutaneously transplanted into C57BL/6 mice. After visible tumor formation mice were intravenously treated with 2×10⁴ PFU of LCMV strains WE, WE-Cl13(L) or P52-Cl13(L). On day eleven after infection tumors were dissected an LCMV replication was analyzed via focus forming assay (FIG. 36).

Thereby it was proven that LCMV strain P52-CL13(L) persists shorter in mouse tumor tissue compared to strains WE and WE-Cl13(L).

### Example 37

FreeStyle 293-F suspension cells were infected with MOI=0.001 of LCMV strains WE-Cl13(L), P52-Cl13(L) or P52-WE(L) at a density of 1.5×10⁶ cells per mL. Supernatant was harvested on 12, 24, 30, 34, 40, 48, 72 and 96 hours post infection. LCMV titers in supernatant were analyzed via focus forming assay (FIG. 37).

Thereby it was proven that FreeStyle 293-F cells show enhanced replication of the LCMV reassortants WE-CL13(L) and P52-Cl13(L) and display an ideal producer cell line for the production of LCMV reassortants.

Embodiments illustratively described herein may suitably be practiced in the absence of any element or elements, limitation or limitations, not specifically disclosed herein. Thus, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group.

Equivalents: Those skilled in the art will recognize or be able to ascertain using no more than routine experimentation, many equivalents to the specific embodiments of the invention described herein. Such equivalents are intended to be encompassed by the following claims.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

Further embodiments will become apparent from the following claims.

1. A method of treating cancer comprising administering to a subject in need thereof an effective amount of a virus particle, wherein the virus particle comprises:
a lymphocytic choriomeningitis virus (LCMV) S segment nucleic acid and an LCMV L segment nucleic acid, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40,
wherein the glycoprotein comprises at least one mutated amino acid residue at position 181 and/or 185 in comparison with the glycoprotein sequence set forth in SEQ ID NO: 4 or wherein the S segment further comprises an open reading frame encoding a nucleoprotein having at least 97% sequence identity to SEQ ID NO: 6,
wherein the L protein comprises at position 1079 corresponding to the linear polypeptide sequence of SEQ ID NO: 40 an amino acid residue other than Lys.

2. The method of claim 1, wherein the glycoprotein comprises at least one mutation selected from Arg 185→Trp and Ile 181→Met in comparison with the glycoprotein sequence set forth in SEQ ID NO: 4.

3. The method of claim 1, wherein the L protein comprises at position 1079 corresponding to the linear polypeptide sequence of SEQ ID NO: 40 a non-basic amino acid residue.

4. The method of claim 1, wherein the S segment comprises an open reading frame encoding a glycoprotein that has at least 98% sequence identity to a sequence set forth in SEQ ID NO: 4 or 14.

5. The method of claim 1, wherein the L segment comprises an open reading frame encoding an L protein having at least 95% sequence identity to the sequence set forth in SEQ ID NO: 40.

6. The method of claim 1, wherein the S segment comprises an open reading frame encoding a nucleoprotein having at least 98% sequence identity to the sequence set forth in any one of in SEQ ID NO: 6 or 16.

7. The method of claim 1, wherein the L segment comprises an open reading frame encoding a Z protein having at least 90% sequence identity to SEQ ID NO: 38.

8. The method of claim 1, wherein the S segment comprises a sequence that has at least 90% sequence identity to a sequence set forth in SEQ ID NO: 1 or 11 or wherein the L segment comprises a sequence that has at least 90% sequence identity to the sequence set forth in SEQ ID NO: 31.

9. The method of claim 1, wherein the virus particle is an arenavirus particle.

10. The method of claim 1, wherein the virus particle is an LCMV particle.

11. The method of claim 1, wherein the subject is a human subject.

12. The method of claim 1, wherein the cancer is a human cancer.

13. The method of claim 1, wherein the cancer is a cold tumor.

14. The method of claim 1, wherein the cancer is a hot tumor.

15. The method of claim 1, wherein the cancer is selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, colorectal carcinoma, hepatocellular carcinoma, testicular carcinoma, colon carcinoma, tumor of the bladder, bladder carcinoma, tumor of the bladder, hepatocellular carcinoma, lung carcinoma, lung carcinoma, endometrial carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, oropharyngeal carcinoma, prostate carcinoma, thyroid carcinoma, cervical cancer, angiosarcoma, chondrosarcoma, Ewing sarcoma, fibrosarcoma, Kaposi sarcoma, liposarcoma, leiomyosarcoma, malignant fibrosis histiocytoma, lymphoma, leukemia, neurogenic sarcoma, osteosarcoma and rhabdomyosarcoma.

16. A method of treating cancer comprising administering to a subject in need thereof an effective amount of a virus particle, wherein the virus particle comprises:
a lymphocytic choriomeningitis virus (LCMV) S segment nucleic acid comprising an open reading frame encoding an LCMV glycoprotein having at least 97% sequence identity to SEQ ID NO: 14, and an open reading frame encoding an LCMV nucleoprotein having at least 97% sequence identity to SEQ ID NO: 6; and
an LCMV L segment nucleic acid comprising an open reading frame encoding an LCMV L protein having at least 97% sequence identity to SEQ ID NO: 40, and an open reading frame encoding an LCMV Z protein having at least 97% sequence identity to SEQ ID NO: 38.

17. The method of claim 1, wherein the virus particle is less pathogenic in a mouse compared to LCMV strain WE.

18. The method of claim 1, wherein the virus particle shows increased replication in tumor cells compared to LCMV strain WE.

19. The method of claim 1, wherein the virus particle shows reduced replication in a healthy organ compared to LCMV strain WE and/or a virus particle comprising the same S segment as the virus particle and an L segment as shown in SEQ ID NO: 2.

20. A method of producing a virus particle comprising cultivating a host cell under conditions suitable for virus particle formation, wherein the host cell comprises a lymphocytic choriomeningitis virus (LCMV) S segment nucleic acid and an LCMV L segment nucleic acid, wherein the S segment comprises an open reading frame encoding a glycoprotein having at least 97% sequence identity to SEQ ID NO: 4, and wherein the L segment comprises an open reading frame encoding an L protein having at least 90% sequence identity to SEQ ID NO: 40, wherein the glycoprotein comprises at least one mutated amino acid residue in comparison with the glycoprotein sequence set forth in SEQ ID NO: 4.
